SEQUENCE LISTING (44 sequences)

SEQ ID NO: 1 - DNA, 13643 bp, Zea Mays
  source 1..13643: mol_type = other DNA
tctaacgccgacaccgtcgacgagtaagaggtcgtggccggctgcccacacgtctgtgtgaacctcaccatcgacaccaccgtccgcctgcggcctacggcctcgaactccaacatcacattcctctacaactgcatgaagaacattaccctaccctctgtcatggaactgagtgggttcccacaacaacaagaagatagatgtaggtcgtacgtgctgtgggatggcgggatcacgggtgctcaggcgtacgggtatgggtgcgaggacttggtggtagcgtcggagctggatgtacacaaaaagggagaaggcgatccacctccggaacggatcgctccatcgggttgccgcatgacgggatcgagctgaactacgacactcactctaagcagtgggacctagggatgaaaacggtcggtaaacactaaagcaattaccgttttcatattttttttatcgaaaacaaaatcgaaaacggtaactccggaaatggaaacgatatcggtatttcggaaacatcgcaaacgaaagttcggtgcgaaaaatacacaagtaacggtcgaaatctaaaatacgatcgataaacatatcaaacttcataatacaacaaagttgacaaaagatcacaaagaccacaagttcacaattcatgatataacaagtttaaaatataacaagttcacaaggatcacaaatttataatataaaaaaattacaaagatcacaagttcacaatataacaagttcacagtataacaagttcataaagatcacaagttcacaaactcaaggttcacaattcacaatatccactcgatttagctgacatggcatgcttactcatgaaatattttttcctcaaataaagagtttttcacagactcgtaggaaaaccctaaaatctagtatgtggaaaagacagactgtcggctctatcattatatattactaatacataacatgtggacagaaaatggtggattcgttcgagagaccaaatcgttaatctcaactgccttccaacaccatctatcaaaaaaaatcttaaggcgtccaaaaaatacaaaaaataagtaatccttatctagagacgtacaggcgatgcgaaaaaaatcacatgctgaataattccgaaaaaaattccggaaaattctgagacataaatccggtaatttccgacaaaaaccggtaaccgaaggaaacagtcggtaaaacaccacgccgattccgataccgactctaatagaaatttccgaaaaccgatttcgttttcgaaaaatactgttaccggtgaatacaatcgaaaaatttcgaaatcggtttccggaataccaaaaaattgtgaaactgttttcatcactagtgggaccgatgtgatggctccgacggatggtgtggctaccagcgtgatgagacgcccaccggcgggatgacattcgcatgtttttgcaaacgcagcccgatcacatgtgcacaatcgttagtggcgcgttttaaaaaagacgtaactacgaaaaaaaatagcgacatgtttttagccagtgtcacaaacgtcatcttcgagcacttttgatgtagtgtatacaatctactagtcaaattaaaaagaataacatacacgaaatggttcataacttttatatagttaaacaataataaatttataaaaatccctaaacaaacaacaaaacgttggacacctccccgctatttatggcggatggcccgcccaattgacgtaatttccacatcgaagtcgaagaaccggtcgctgtgaccagtcaccccaacctccgtcacttgtctctcacgcgcgcgcaacatcgccgagaaaaccagaacagacctccggattggtctctcccccgttcacacgagcacattgcgatggctcctctgctcctgctgctcctcttccccgtccagctccccctcgcagtagccgacgccgtctccggtccctgcaccagagccacatgcgccggccaggacgtccattacccgttctggctcaagtcctccgcgcccgactgcgtctatcccggtgtcggccttgtggcccttgtctgcgagggcaactcgacgctgatcctccccttcaagtcccacagatacgtagtgctcagcatcgactacaagacgcgtaccgtgctggtctccgacgccgacatcgtccacgagtacgacgcggccggctgcccgcgcgtccgcgtgaacctcaccatcgacaccgcctggctgcggcccacggcctccgactccaacatcaccttcctctacaactgcaagaagaacatcaccctgccctccgccgtggaactgagcgggtgccagcagcagcagcaacaagacggcagcaggtcgtattcgtacgtgctgccggacggcggggtcacgggcgctgaggcgcaccagtacgggtgcgaggacttggtggtggcgccggtgctggacgtccacaggagggcgatcttgagggcgcctggcggcccgactctggagaacgggtcgctccgtcggttgctgcagggcgggttcgagctgaactacgacactcactccgagcagtgcgaccgatgcgaggcctccggcgggtggtgtggctaccagcgcgacgagacgcccgccggctggatgacgttcgcctgcttctgcgacggcgggccgacgaccacggcccgatgcggtgccggtatgtcttttttttttcttcgaacaaggtgtggcatgtgttctaccgtttcaactagtaaatgattacattgagctaggcagctagccacacattttcttgaatgattttctttgatgaacctgctgtttgcttttatgacgtgaacaacggggcctgcaagctgccacatacctagggagactagttcgtgaccttctttacacgtcttctctactcgccactgggagttgacgccgctcggtcgtcccactttgtgacgttcaaccagagtctagagatgtaattctctgcgaatacaggactagttggagctaacaacgcagcttgacgagggtgaacccagctccacctcgtctaccacgtcttctcctcgccatggctgctcacctaccacgcctccccgtcctcctcctcgtcctcctcgctgctcatgtcgtctccacctccgcccatgccgagcctcctcttccgagcccttacagcacctccgcccatggcgagcctcctcttccgagcacttacaacgtctccatgtgctcggaatcgttctggtgcggcggcgtcgaaatccgctacccgttctatcttgccaacgcaaccgccgactacagcgggagctactactcctgcggctacaccgacttgagcgtttcctgcaaactcgaggtcgaggggccgacgacgacatggacccctaccatccgtctcggcggcgacaactacaccgtcaagaacatcttgtacgactatcataccatctcactggcggacagcgatgtgctcggaggcggcgagtgccccgtcgtccaccacaacgtcagcttcgacgagacgtggctgcacaaccccagcgccttcgacaacctcaccttcttcttcggatgccactgggggccacgcgatacactgcctgaatttgccggcaacaacatcagctgcgccgggttcagtactccagctatcagcggtggaggctccttcgtgttcaagcctgaagatcttgacgaacatgcggagcaggagttggcttcacactgcgacgaggttttctccgtgccagtgagaagcgaggctctgcagcaggcgatcgtcagcaacctcagcctcggggacgggtacggcgagctgcttaggcaggggatcgagttggaatggaaacggacatcggaggatcagtgtggccagtgcgaggaatcgggctccggcggacggtgcgcctacagccagaagagagaattccttggctgcttgtgcagcggagggaaggcgggcaacccgttctgcaaaccatcaagtaaagtcctgaaccgagcctcccttatttttttttcattttttgcaatccaccagagagcacgcatcggttgcgtcagtatcttgcaacctcgtagctagccccgcagtgtcccctgtgtgcgagtaccgcgctgctccagcttgcctcctgctaacgcctaacggtgaatgcttcatgcttgacatgatctagctagtctacactttgcttggggtttgcctgggagctggaaattctggctcctgtttgcatcactcgacaaggacgctttcagacttgcgactctcgttctgcttttgcaccaaatccgtgtttttttcatttcgtgatcgagattaatctagcttagagatgacaatgggtatccgaaactcgaaacttgatggatttttactccattagggtataggtttgaatcaattttcatatttatggatttgttaataggcataaatatatatccaacaggtttatagatacgagtttgtttctacagtactcaaatccgtgaacacatgaggtttttaaacccgaccaaacctagtgcatattgtcattttattttataaacgaacaacaaaattgttatctctatttacttcctattttttatcgattggtgaatgtataagtagttggtgagaatgtttcttgcttgctattatagttttactagcgttatatatgttgtgggtggataacttagtgcaatgtcacttgattatacaacttattatttgtattcattctctctactaataatttttataccaaatcatgaactcggtgtttattatataaattttggaccataatctcattaatcatcacgatagttattgattatgagaaaaaacaagcatattggagataaaaccctcggctaacccgttaacccgatgggtacgggtttgaacaaaatttcaaacctattatgaatataagttttttaacaagtatagatatatttcacggatagagtttaagatgacaaaatccaacggatttgtatccattgccatctctatccggcggcccttacccggcggcccttaccgtgctccacgagcagaggtcgtatcgtccctcttcccgtgtcgcctgcttcgcgttgccgaacggagacgtttggtagcgttggccggctctagcagtcgggtcaactctttttgttgttgttttcgatgttgttggatttttgttccgtataagccatgttttagtaatttatttagtccagccgaatccgaagacgtgtttgctgggttggagactttggagttgctagtcatgatatgctttctactcggtttgatttcaacccagttaggctatatttaatactctagtatttatttcaatataaatggtttgaaacggattaaggtataaattagtttaatttatatatttaattcctctcaatccatatgtattgggctgaatactgaggtagtgtttggttgaagagccatatagaacggagccgttctgttccagttttgttgttgtttggttataaagtaactagaacggaatgacttcaattaaggaatattcttctcagatccagaaccattccgctctaaaaaatcaaccggacggagccgctccgtttccatcctgctcttacagtcacgctccgttccgttcactctgcaaccaaacaaaaaacagagccgctccgttccaaattaccaaacacagaacagagcggcttcattcctagaattaggaatggaacgactctgttctacttgactcctcaaccaaacactacctaagtatccaaacaagcccttatttaagatgcatttcctttacagttacacatgaccactattgtgtgggggcaggctgaacaagcccttatttaagatgcatttcctttagagttacacgtggccactattgtgggggggggggggggggggcaggccgatcctactcgtcagtgctcattcgagagcaaagataccgaaggagattagagagactaaaaactttttactatttaaaattagataataagacgatttaatcccgttccatatctttgctctaaacaaaccctcaatgatcatatatctcggaagatccggccggctgttctttatttatcaagtgatgactgctgaccgcttatagaatatatattttaaagcaaaatttcttctacagcagtaaaaggactagacgaaacaatgatgcatttctctaacaaaagaaagtagaattatcaagcggagagccaagaccaaaagccttacttctatgggcgtcaacaaatgataccgcgacggaaccatcccagcaggtctatactgtctgtcacgacccagcgagtaatcgtgtggctacgctattagacttaggattggatgaaatgctcggtttattaatgagccagctcgtgagttaagagtgtttggtttgatgaatgaagtaattcatcttcttttcactccccacttttttatttggtttgtgtaatagaatgagttgatccatcaccaccacattcatcataagctaataattagtatatacatgagtagtgagttgattccaccaaaattgatgaaatgaacttatgatgcatcatctcatgaagcatagagtgactccacaaaccaaacacaccataaatgatctattacataacgaaattatatgcatatcatttatcagggcgacgacagggggcatagggaggcaatatccccctaatgctccccaaattctatagggattgttagtttcttttagctaattctcatgtaaacaatataaaaaatgcttctaacagtccctcctaattataatttggtccaccctaatcttagatcctggcttcgtccgtgccatttacgtaacaattggtaaatatgttatacatgtgtggtatctatggcctatgaattgaactaatgattgatgaattgtgcttatgtgttaaattggtctatgcgaatataactatgggttaaacggatgaacatgtgtgttcattgttaattcatgagtgatgaattatgtataatttggtgttatattgatgtgttttgtgaaactatgtgtataattattattttctataattaaatttgtttgaaattaactagaaattgattattatatatatatatatatatatattgtttttctgctctagtctgcaagctaaacgagcaagctcaagctcgtaaacgatccgaaccgagctgactttgtggctcattaacttaacaagccgagttgggccaacttgttagcttaacgagtcagctcgaatacggacaagttgagccgagttggcatgatatccagccctaattaggcttgtaccagtgcaacatatccctctcgcctttgtcacgtccagacatgtcaatgggccccgattcccgcaaggaatttctctattaagggatgaggatgggaaagtttctccccccacagaaaaattctctcccgacggataagcggggacaacactccccatccccattccccgtggggacctattagacttacatatggtgatgttttcatgtaaaagttaatgataaaaataaacaattaccttgttgtcggcgtttcgaccccggagggtccctggaccgacgagtaaattgtcgctgcgtgtcccagcccagatgggtcgacgcgagacggaacacaagggggaaacaataaggggaatcgcggcctcgtgttgtcctgcgcccagggcggatgcgcttgcagtaaggggttacaagcgttcgcgagggagagagagagagagcctgtgcgccagcccgtcctcccgcgcggccaccttctcgtacgagggccctggacctttcttttatagatgtaaggagagggtccaggtgtacaacagggagcgtagcaatgtgctaacgtgtctagcagagggaagccagaatcctatgtacaggccgacgtgactgtcgaagaggttttggcgccctgttcatgtgatgtcgtggccgtcggaggagcgcttgagccctgtaggagcacagctgtcggagctgtcgggtccttgctgacgtctcattgcttccatagggagctgagaaccgccgtcgtcatggagcacgcggggtgccatcattacttgttttaccgggacgagccagatgggacgctggtcttgttcccagtagcctgaggtagctagaggtagggtaatgatgtgccctcctgcgacgtggtcggtccgagcccaaggtcgggcgaggcggaggctcctccgaggtcgaggctgagtccgagacctggggtcgggcgaggcggagaccgtcgtccgaggtcgaggttgagtccgagccctggggtcgggcgaggcagagtccatcgtccgaggtcgaggttgagtccgagccctggggtcgggcgaggcggagacagtcgtccgaggtcaaggttgagtccgagccctggggtcgggcgaggcggagttcgtcttccgaggtcgaggtggagtctgagccctagggtcaggcgaggcggagaccgtcgtccgaggtcgaggttgagtccgagctctggggtcgggcgaggtggagcttcctatggcgcctgaggccggacttggcggctgtcagcctcaacctgacgggtggcacagcagtcgaagcagcgcaggcggcgctgtttttctatcaggtcagccagtggaggggcgaagtgactgcggtcacttcggctctgtcgactgaagagcgtgcgtcaggataaggtgtcaggcgatccttgcattgaatgctcctgcgatccggtcggctggcgaggcgatcttggctaaggttgcttctccgcgaagcctgcctgagctgggcctcgggcgagtcggaggtgcgcccgttgcttgaggaggccctcgggcgaggcgtgaacctgcctgggcctgctgtttctgcccgaggctgggctcgggtgaggcgagatcgtgtcccttgagcggacagagctttgtcctgtgttgcgcccatcaggcctttgcagctttgtgctgatggtgtttaccagccgagtttaagagtcttgggggtacccctaattatggtccccgacacttgtcaagagatcactttttgtacaaatatattcattctgatgtacacatatttttttcttacatctaacaatgtgtataagtgagaatgttttatattaataacaaatgaagtatgtcctaattagacttctcacattgagcagcaacaaaacattttatgaattagtaaccattttacttactaaaataattactattgtatcttaatcattttgtgcaaaataaagaatgaaccaaattttgggtcaggtgtgggtcactcgcaggttgaaataaaaacccgcacccacactcgtgaaactttgggtcagatgcgggttacacccgcggattaaaatctctacccatacccacactcatcagatcgagtacccaaagattttaagtttgcgggttaaattgtcatccctcgacaccaacgtgttggaggatttggaagtttcgcgatgcgtacttaccaagtgtttggttctatgataaaagtttagcctgtgtcgcattagatgattgaatgtctattatgagtattaaatattgtctaattattaatcaaattacacaagtgaaggctaaacaacgagacaaatttattaagcctaattaatctatgattagcaaatgtttacagtagcaccatctagtagcaccatctgagcgaatcatgaactaattaggtttaataggttcgtcttaacgtttaattcttatctatgtaattagttttataagtaaattatatttaatacttctaattagcctccaaacattcgatataacatagactaaaatttagtcagtggtgctgctgctggctcttttccttcatccgacgtaatgttttcctcccaatttattgtgttgttttgtttgtgctttaaaactgacttgctctctgcttgctgtatcaaagtcgtgatgtgtgcttagcttattcccttccttgctagctgcccttagcatctatatagggatatttggttatagggactaaagtttagtttagtccgtgtccaattataaaactaattacataactgaatactaaaagacgagaaaattttattaaacttaattaatttatgattagcaaatgtttattgtaacatcacataagtaaattatagactaattttgtttaataggttcatcttatcatttagtcttcatctatgtaattagttttgtaattagactatatttactatttctaactagtatctaaacattaaatgtgacatggattaaaatttagttagagcaactccagtagttttctaaaagacttcctaaatcaataatttaggtagttaacatgaaaactattctccaacagttctctaaataaactttctaaatttaacaacttgtcatctaacctcattttctctctacatttggcaaccatttaataactccctaatcaaaaatgttgactgcattatatagtttttgtgacttattttttatgtggataaatacaaaataaaattacaacctatatttagagaactattggagaacccacttatttttatttcaaaagtcatttagcaacttcttaaatctgtaatttagaaagctaaaatttacataactattagagttgctcttagtgacgttaggcttttagagttgagttggtcgtagcttggtttagttacgtgtttgttctcttgcgtttatttaggacctctctataatgttttatcaccttcttaatacaaattaaaatacgcagctctcttgcgtattggaggcgtgtgttcctatattttttaggctcaattatgaatgaaattattctcagcgagcatataaccgttttcgaggtaaaatgaactaaaagcatatgggcctgcctgttcgcataagtacaccctccgtctaaaaaagaataaaaatatcatttcttgatgagtcaaaaaagttcaaatttaagaaaatatatgttacgacaccaatatttataatgtgtaataagtactgctgattaattttaaaataaaattttcataataaacctatttgaagatacaagtattggtactatttctaataaatctaatcaaactggtgttatatcttttgtaacaaatttgtgctttgtgtttctggttgacgtgaatcagcttaatcttgctgaaatctaacattgtcttttgttcgttggcatacaggatcaaaaaggaaagaagcatctattgttggtaagagcctatagtcaatacccatgttcatttcgtctaaaagagcagaagaaaagcatatgatgaattattgccatgtcatgtttaaaatacagaattctcaaaaacaaaaacaaaaaaaacttggaatccactaaccagtaaccactgatagcattgtagaaaatttcatcctccctttgggcaatacactgatgagtttacatgctgactagtggtgcatttgttctttgccaattgaatttttagaatgctttgcagctgaattcacttgtgatttttttttgtgatgcaggtgctgttgccgttgcattcctgtgtctagtcattctcacatgcttcttggcttgtagacatggttcgctgcccttcaaatcggagaacaaaccagggacaaggattgagtccttcctacagaagaacgagagtatacatccgaaaagatacacctacacggacgtgaaaagaatgacaaaatccttcgctgtgaagctaggccaaggtgggtttggtgctgtatacaaaggcagcctccacgatggccgacaggtagcagtcaagatgctcaaggacacccaaggtgacggcgaggaattcatgaacgaggtggctagcatcagcaggacttctcatgtcaacgtcgtgacacttctagggttttgcttgcaagggtcgaaaagagcactgatctacgagtacatgcccaatggttcgctcgaaaggtatgccttcaccggtgacatgaacagtgagaatttgctaacctgggaaaggctatttgacatagcaattggcacggccagagggctcgaatacctacaccggggatgcaacactcggatcgtgcattttgacatcaagccacacaacatcctgttagaccaggatttctgtcctaagatctctgactttggactggccaagctatgtctgaacaaagagagcgctatctccattgttggcgcaagagggacgatagggtatatcgccccggaggtctactcaaagcaatttggaacaatcagcagcaagtctgatgtctatagctatgggatgatggtccttgagatggttggagcaagggaaaggaatacaagcgcaagcgcagatagtgaccatagcagccaatatttccctcagtggatttatgaacatttggacgactattgtgttggtgcttccgagattaatggtgagaccacagagctcgtgaggaagatgatagttgtaggtctgtggtgcatacaagtgattccgactgatcgaccaacaatgacgagagtcgtcgagatgttggaagggagcacgagtaatctagagttgccacccagagttctcttgagctgacaaagcgtagatatttttcctatcaaatgttgcttccaggtcacacaaatgcaaaatatttgtggagacgagtgcctatttacctcatacactgtatctgtatgacaaaagtcccacgactcactggacgcggaaatgtcgcttgactacgccaattttctaaaaagattggcagcaattaatggaggcttatagcggtaactttggttcgcattaatcctaggactagggttgaatatcgatctaactcgacgcggcttggtcaagctcaagctagctccactcatctcattaaagaatccagctagaaaatcaacccaagtcgtttacgaaacgagtttgagctgactcgtttagatcgtaaatcacaacaaaaacaatatgcacatatatacaataatataatcaatactagttaattctagactagtttaacactagaaaagagtaatgatactcataatttcacatacaatgtcaatccaacaccaatttaacacacttcatcacttattagttcatccaaccaagtgtaggctttgatttactaacaaatggttgctcgttcgagctagcgagcttgcttgttaacaaactgagttgagatgctagcttaacttgtgacaaaattaaaacgagccgagtcgagtcaagttgagctcacgatgagtcgagcaagctcacaatccacgagtatttttttagtcctatctaagactaaagtttaatcctaaaactaaattttaatctctatttgtttggttctataaactaaacaggttcagaaaacataaaatacattatagaaaacctgaaatacccttctatacttaaggcatcactaagagagagcaataaataaagggtagagagaggaataaatctgctttattcccttttagctaccctttgagagagtaaacactaaaatgaaaggatccttgaggattttgatgttttggatgacaactaacacaattaaaggtctaattaggatgttaa

SEQ ID NO: 2 - DNA, 2001 bp, Zea Mays
  source 1..2001: mol_type = other DNA
atggctgctcacctaccacgcctccccgtcctcctcctcgtcctcctcgctgctcatgtcgtctccacctccgcccatgccgagcctcctcttccgagcccttacagcacctccgcccatggcgagcctcctcttccgagcacttacaacgtctccatgtgctcggaatcgttctggtgcggcggcgtcgaaatccgctacccgttctatcttgccaacgcaaccgccgactacagcgggagctactactcctgcggctacaccgacttgagcgtttcctgcaaactcgaggtcgaggggccgacgacgacatggacccctaccatccgtctcggcggcgacaactacaccgtcaagaacatcttgtacgactatcataccatctcactggcggacagcgatgtgctcggaggcggcgagtgccccgtcgtccaccacaacgtcagcttcgacgagacgtggctgcacaaccccagcgccttcgacaacctcaccttcttcttcggatgccactgggggccacgcgatacactgcctgaatttgccggcaacaacatcagctgcgccgggttcagtactccagctatcagcggtggaggctccttcgtgttcaagcctgaagatcttgacgaacatgcggagcaggagttggcttcacactgcgacgaggttttctccgtgccagtgagaagcgaggctctgcagcaggcgatcgtcagcaacctcagcctcggggacgggtacggcgagctgcttaggcaggggatcgagttggaatggaaacggacatcggaggatcagtgtggccagtgcgaggaatcgggctccggcggacggtgcgcctacagccagaagagagaattccttggctgcttgtgcagcggagggaaggcgggcaacccgttctgcaaaccatcaagatcaaaaaggaaagaagcatctattgttggtgctgttgccgttgcattcctgtgtctagtcattctcacatgcttcttggcttgtagacatggttcgctgcccttcaaatcggagaacaaaccagggacaaggattgagtccttcctacagaagaacgagagtatacatccgaaaagatacacctacacggacgtgaaaagaatgacaaaatccttcgctgtgaagctaggccaaggtgggtttggtgctgtatacaaaggcagcctccacgatggccgacaggtagcagtcaagatgctcaaggacacccaaggtgacggcgaggaattcatgaacgaggtggctagcatcagcaggacttctcatgtcaacgtcgtgacacttctagggttttgcttgcaagggtcgaaaagagcactgatctacgagtacatgcccaatggttcgctcgaaaggtatgccttcaccggtgacatgaacagtgagaatttgctaacctgggaaaggctatttgacatagcaattggcacggccagagggctcgaatacctacaccggggatgcaacactcggatcgtgcattttgacatcaagccacacaacatcctgttagaccaggatttctgtcctaagatctctgactttggactggccaagctatgtctgaacaaagagagcgctatctccattgttggcgcaagagggacgatagggtatatcgccccggaggtctactcaaagcaatttggaacaatcagcagcaagtctgatgtctatagctatgggatgatggtccttgagatggttggagcaagggaaaggaatacaagcgcaagcgcagatagtgaccatagcagccaatatttccctcagtggatttatgaacatttggacgactattgtgttggtgcttccgagattaatggtgagaccacagagctcgtgaggaagatgatagttgtaggtctgtggtgcatacaagtgattccgactgatcgaccaacaatgacgagagtcgtcgagatgttggaagggagcacgagtaatctagagttgccacccagagttctcttgagctga

SEQ ID NO: 3 - protein, 666 aa, Zea Mays
MAAHLPRLPVLLLVLLAAHVVSTSAHAEPPLPSPYSTSAHGEPPLPSTYNVSMCSESFWCGGVEIRYPFYLANATADYSGSYYSCGYTDLSVSCKLEVEGPTTTWTPTIRLGGDNYTVKNILYDYHTISLADSDVLGGGECPVVHHNVSFDETWLHNPSAFDNLTFFFGCHWGPRDTLPEFAGNNISCAGFSTPAISGGGSFVFKPEDLDEHAEQELASHCDEVFSVPVRSEALQQAIVSNLSLGDGYGELLRQGIELEWKRTSEDQCGQCEESGSGGRCAYSQKREFLGCLCSGGKAGNPFCKPSRSKRKEASIVGAVAVAFLCLVILTCFLACRHGSLPFKSENKPGTRIESFLQKNESIHPKRYTYTDVKRMTKSFAVKLGQGGFGAVYKGSLHDGRQVAVKMLKDTQGDGEEFMNEVASISRTSHVNVVTLLGFCLQGSKRALIYEYMPNGSLERYAFTGDMNSENLLTWERLFDIAIGTARGLEYLHRGCNTRIVHFDIKPHNILLDQDFCPKISDFGLAKLCLNKESAISIVGARGTIGYIAPEVYSKQFGTISSKSDVYSYGMMVLEMVGARERNTSASADSDHSSQYFPQWIYEHLDDYCVGASEINGETTELVRKMIVVGLWCIQVIPTDRPTMTRVVEMLEGSTSNLELPPRVLLS

SEQ ID NO: 4 - DNA, 1887 bp, Zea Mays
  source 1..1887: mol_type = other DNA
atggctcctctgctcctgctgctcctcttccccgtccagctccccctcgcagtagccgacgccgtctccggtccctgcaccagagccacatgcgccggccaggacgtccattacccgttctggctcaagtcctccgcgcccgactgcgtctatcccggtgtcggccttgtggcccttgtctgcgagggcaactcgacgctgatcctccccttcaagtcccacagatacgtagtgctcagcatcgactacaagacgcgtaccgtgctggtctccgacgccgacatcgtccacgagtacgacgcggccggctgcccgcgcgtccgcgtgaacctcaccatcgacaccgcctggctgcggcccacggcctccgactccaacatcaccttcctctacaactgcaagaagaacatcaccctgccctccgccgtggaactgagcgggtgccagcagcagcagcaacaagacggcagcaggtcgtattcgtacgtgctgccggacggcggggtcacgggcgctgaggcgcaccagtacgggtgcgaggacttggtggtggcgccggtgctggacgtccacaggagggcgatcttgagggcgcctggcggcccgactctggagaacgggtcgctccgtcggttgctgcagggcgggttcgagctgaactacgacactcactccgagcagtgcgaccgatgcgaggcctccggcgggtggtgtggctaccagcgcgacgagacgcccgccggctggatgacgttcgcctgcttctgcgacggcgggccgacgaccacggcccgatgcggtgccggatcaaaaaggaaagaagcatctattgttggtgctgttgccgttgcattcctgtgtctagtcattctcacatgcttcttggcttgtagacatggttcgctgcccttcaaatcggagaacaaaccagggacaaggattgagtccttcctacagaagaacgagagtatacatccgaaaagatacacctacacggacgtgaaaagaatgacaaaatccttcgctgtgaagctaggccaaggtgggtttggtgctgtatacaaaggcagcctccacgatggccgacaggtagcagtcaagatgctcaaggacacccaaggtgacggcgaggaattcatgaacgaggtggctagcatcagcaggacttctcatgtcaacgtcgtgacacttctagggttttgcttgcaagggtcgaaaagagcactgatctacgagtacatgcccaatggttcgctcgaaaggtatgccttcaccggtgacatgaacagtgagaatttgctaacctgggaaaggctatttgacatagcaattggcacggccagagggctcgaatacctacaccggggatgcaacactcggatcgtgcattttgacatcaagccacacaacatcctgttagaccaggatttctgtcctaagatctctgactttggactggccaagctatgtctgaacaaagagagcgctatctccattgttggcgcaagagggacgatagggtatatcgccccggaggtctactcaaagcaatttggaacaatcagcagcaagtctgatgtctatagctatgggatgatggtccttgagatggttggagcaagggaaaggaatacaagcgcaagcgcagatagtgaccatagcagccaatatttccctcagtggatttatgaacatttggacgactattgtgttggtgcttccgagattaatggtgagaccacagagctcgtgaggaagatgatagttgtaggtctgtggtgcatacaagtgattccgactgatcgaccaacaatgacgagagtcgtcgagatgttggaagggagcacgagtaatctagagttgccacccagagttctcttgagctga

SEQ ID NO: 5 - protein, 628 aa, Zea Mays
MAPLLLLLLFPVQLPLAVADAVSGPCTRATCAGQDVHYPFWLKSSAPDCVYPGVGLVALVCEGNSTLILPFKSHRYVVLSIDYKTRTVLVSDADIVHEYDAAGCPRVRVNLTIDTAWLRPTASDSNITFLYNCKKNITLPSAVELSGCQQQQQQDGSRSYSYVLPDGGVTGAEAHQYGCEDLVVAPVLDVHRRAILRAPGGPTLENGSLRRLLQGGFELNYDTHSEQCDRCEASGGWCGYQRDETPAGWMTFACFCDGGPTTTARCGAGSKRKEASIVGAVAVAFLCLVILTCFLACRHGSLPFKSENKPGTRIESFLQKNESIHPKRYTYTDVKRMTKSFAVKLGQGGFGAVYKGSLHDGRQVAVKMLKDTQGDGEEFMNEVASISRTSHVNVVTLLGFCLQGSKRALIYEYMPNGSLERYAFTGDMNSENLLTWERLFDIAIGTARGLEYLHRGCNTRIVHFDIKPHNILLDQDFCPKISDFGLAKLCLNKESAISIVGARGTIGYIAPEVYSKQFGTISSKSDVYSYGMMVLEMVGARERNTSASADSDHSSQYFPQWIYEHLDDYCVGASEINGETTELVRKMIVVGLWCIQVIPTDRPTMTRVVEMLEGSTSNLELPPRVLLS

SEQ ID NO: 6 - DNA, 5930 bp, Zea Mays
  source 1..5930: mol_type = other DNA
gatattgaatccaattcaattctataacctccaaaatcgatatcctaacatagcatgatagtttttttgagaggtgtgatttaacatattagatgaattttctttttttaatacgctatgttctagatgtctgacgatgaatggagccgctctgcatataccaaacgctatctaagtttgtgaacaaatgactaaattatccacacgaccacaattgggcgctgataagatccacacttgctggttttaattcatccggtctttatcaactgtcacatcagttatagatcattatcaacttttatctacaattgatgttgtaatacacggtcacttaagaccatgtttaggtacaatgtctctcaaaactatgattttacttatgatgacaataccgtagttttgaatagctctaaaaatatcatggttctaaaaatattgtttggattcaacattataaatcgtggtattaagcaaaagctagtcatgttataaaaactttaggttgaagtagagttttcaatactaaaaaaatatcatggtatttagaatatcatgattttaaaaatatagttcttacgaatgcaaccaaacaccttatgatataaaataatatggtattgcctacaaactacaaaaataaatcactcccaagcattacataaacatcatttttataaactttttagtaaaataaagcgcaatgtttttattaaatagaaatttatacaagtatatataattaagcaaataaaaactacgttattcataaaaagctaataaaaagtaaaaagtaaaaactatataattcataaaaaaatcgttatctttctcttccatcttattatctatttttagttgttttgtcaaatttaaggctattttatgggttctttttatctttgtacatgtcgtgagccaataacttattgaattggtcaatcaatttaggttgagttttacttttaactaacctagtatagtttgttcaacacctttttttcataatcggggactcattccacttgttgttgacccattttttcttctactgagttcttgcccttttgcaatcaataggacgtggtgagcccatatttttaggtgttatggatttataatcaccgttcaaatttgaatgaatattttgtttttattattgaagtcttcgtcaatcgttatcatgcctcgcataccacttagaccgacgaagtccaacatatttctcccccgacgaggagagattcttcgtaaaattgctactggtattactggtattgaatagaagataagggtgcgtttggttgcatgcatctacgcgtgcttgcatcacggaatgcgggttggtgctgtttggttgctataagctagactatacatatgcaagttttgtgtttggttggctgcatgcagatttcgatccaaactcgcacgatccacacagcaccctgggccaggcttaccagatacgagtagattggtcgcatctttggagccaggctttagcggtccgcggtcctggctgacacggccagattgccgagagaaaccaaccaaacagggtctaaatgtccaatcgtttgactgctactggatgaaaaaagaatgtcacaactttaaaatgtgtatttatttatactcctacacatgaaataactactatactcagatttcttttacattcagattttttctcagccactgaaacataccagccctttactaccaaaaacaggaactccacggtccaatgattatgtgagtcggaggagaggggaggaagaatcgcctgtgaaggagggagggagggagggacgtccgatccgaagatggaaggagctggggaggggagcgccttgacggggatgatgggtcctgcgctcgacaagctcgctagcctcgttgacaagtacaccgagctcagaaacgtgaggaagaagatggagcagctgaggaaggagctgattgcgatcaacctcgcgcttgagaagcacgcggccatggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgttcaggagatcaaacaactccacgaccttgccaacgaatcgtaccggcgtaggaagaggtacaggattgaggagggcggttcaagcctctcgcacgcggagatcgatcctcggttagaggcgctctacgtggaggtggagaaactcgtgggcatccagggcccaagccaggagatcattggacagctcgtcggcgagaacgcagcggagcgacggagggttgtcgccgttgttggatctggaggttcaggcaagaccacacttgccaaacaggtgtacgagaaaatcaggtgccaattctcttgtgcagcctttgtgtctgtgtcgcaaaagcccaacatgaatagcctcctgtgggagttgctatctcaaatcgggaaccatggtggagatttaggaatgatggcagtaggatattgcagtgacaaacaactgatcgacagactaagatcacatcttgaaaagcagaggttagtttaccttttcattccggttagcttaattcggtacaccaactagagatttgtgatttgctattaattacaccaaatttctcctacacaacaataactggtttagcatgatggcgatccaaagtcaaaactatcttctactactagtgtatgccatactcatatagatattttcttttcataaactctcgtagcatttttacatgcattcatattcctattgcctttatacagaactgatttttcactgcttcacaatctgctcttaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctatgcacaagaaagatagtggcctggaagtgatggacaagatactgtctctgagttacagggatctacctcacaacatgagaaattgcttgctgtatctcagtacatttccagaggaccacacgatttacaaagatgccctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgacgaacctgactgcattctaagcttgtttcatctgagatacctgggtttccgcagcgcaagtggtgtcgtgctaccggctcaaatcggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctagcacttctgagcaggctgagggtgctccgagtggctttcatcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttgcctataccttttcgtgaagcgagtctccgaagacgggctcgccatcagccacggcgcgttccgatgtctacggcgtctcgagttctgcaacgtagatggacctggtttgatgtttgcaggaggcgttccaatgttggaatggctgaggctcgggttcgacgcggatagagcgcaatcgacatacggcggtctggaggttggcatccagcgcctctcgtctctcaaacatgtcgtgctcattgtatggatggtttctgaaggcggtgatgatccagcggagcaagccgtctggtctgccatcaatggccaagtagagatgctccccaactctccgacggttgatatccggtttcgtagacggagtcagctgcaggcaagctcagaataaggagcacgaaaaagacgatgatgttggatgtcgcctgctagctgtagtatgttgctgcttctgcttgttgccaacacattttttttgggttagggtggggtacaaccataaaatgtgtgtggatgtgcttgtaagcattacttgtatgtttttttttgtaaagcacaatatagatagatgcatatatgtgtgcgtgcaaagctatgattatcgacactcacacttgtacattagctagatgaaggtctcgacagagcagagcatagtacacatctctgggagttggactggacactataacggggatgctgcagccgaaactcaaaagctacatgcatgtcacttggctcatcggcgcaggaagcaacgcaggtccatcacgcgcagcaccttgaacccgtccaggctacaccccagcagcagcctcggcttcttcggcgccggcgggcagcgcggggggcgttcggttccagcgccactcgccgtcgccgacgacagcggcgagagcttccgtggaccttcttcgtcctcgtcgccgccgcgggagtaagaccgcttcagcttcagcggccgcagctccaccagctgcggaatgctaacatcttcttcaggcacctgccgggtggttctctttcccagaatcttccgctggagacccatggcgagctcggttcggtttaacctgcaattaatgcgcaatagcggagtaggaagcgagattgttagctcaggcgagcagagatgaaccggctaggaaagtttagttgctcgtgcatgctaccagctagcttgttggaggctcttgctttggacaccaagcaacggatatgacagcagaatgtgcgtgcttataagcaagcaagcagagcagcagttgcaaagaagggaaaaggtggagtggaaaaaggagttgccattgaagtgcacgagtgagcgagcatcatgtcaaggaaccaggagggaagttgcacaacagatgaaaagcggagagctgtttccg

SEQ ID NO: 7 - DNA, 2829 bp, Zea Mays
  source 1..2829: mol_type = other DNA
atggaaggagctggggaggggagcgccttgacggggatgatgggtcctgcgctcgacaagctcgctagcctcgttgacaagtacaccgagctcagaaacgtgaggaagaagatggagcagctgaggaaggagctgattgcgatcaacctcgcgcttgagaagcacgcggccatggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgttcaggagatcaaacaactccacgaccttgccaacgaatcgtaccggcgtaggaagaggtacaggattgaggagggcggttcaagcctctcgcacgcggagatcgatcctcggttagaggcgctctacgtggaggtggagaaactcgtgggcatccagggcccaagccaggagatcattggacagctcgtcggcgagaacgcagcggagcgacggagggttgtcgccgttgttggatctggaggttcaggcaagaccacacttgccaaacaggtgtacgagaaaatcaggtgccaattctcttgtgcagcctttgtgtctgtgtcgcaaaagcccaacatgaatagcctcctgtgggagttgctatctcaaatcgggaaccatggtggagatttaggaatgatggcagtaggatattgcagtgacaaacaactgatcgacagactaagatcacatcttgaaaagcagaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctatgcacaagaaagatagtggcctggaagtgatggacaagatactgtctctgagttacagggatctacctcacaacatgagaaattgcttgctgtatctcagtacatttccagaggaccacacgatttacaaagatgccctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgacgaacctgactgcattctaagcttgtttcatctgagatacctgggtttccgcagcgcaagtggtgtcgtgctaccggctcaaatcggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctagcacttctgagcaggctgagggtgctccgagtggctttcatcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttgcctataccttttcgtgaagcgagtctccgaagacgggctcgccatcagccacggcgcgttccgatgtctacggcgtctcgagttctgcaacgtagatggacctggtttgatgtttgcaggaggcgttccaatgttggaatggctgaggctcgggttcgacgcggatagagcgcaatcgacatacggcggtctggaggttggcatccagcgcctctcgtctctcaaacatgtcgtgctcattgtatggatggtttctgaaggcggtgatgatccagcggagcaagccgtctggtctgccatcaatggccaagtagagatgctccccaactctccgacggttgatatccggtttcgtagacggagtcagctgcaggcaagctcagaataa

SEQ ID NO: 8 - protein, 942 aa, Zea Mays
MEGAGEGSALTGMMGPALDKLASLVDKYTELRNVRKKMEQLRKELIAINLALEKHAAMENPDAQAKAWAAEMRELAYDMEDSIDLFTHHVDHEPADTATTGVKRFFLRIIRKLKKLHYRHRFVQEIKQLHDLANESYRRRKRYRIEEGGSSLSHAEIDPRLEALYVEVEKLVGIQGPSQEIIGQLVGENAAERRRVVAVVGSGGSGKTTLAKQVYEKIRCQFSCAAFVSVSQKPNMNSLLWELLSQIGNHGGDLGMMAVGYCSDKQLIDRLRSHLEKQRYLVVIDDVWTNSAWETIQCALPKNAHASKIILTTRINSVGQFSCTPDEGFIYQMKPLCRNDSENLFLKRTLCDKDKFPAQLEGIKNEIIEKCDGLPLAIVTLASMLATKQRTREEWERALDSIHSMHKKDSGLEVMDKILSLSYRDLPHNMRNCLLYLSTFPEDHTIYKDALVWRWMAEGFIAETQGFTLEQVAEGYFYEFVNRSLVQPITLRSRYEMRGEGGCRVHDIVLNFLISRAAEENFLTTLYGAQGVPSSDRRIRRLSVWDSPEHALAVSRATMNLSHLRSVRICNVGDWPVPAVLDLPVLRVLDLEGCRDLRIDEPDCILSLFHLRYLGFRSASGVVLPAQIGNLHHLQTIDLSGTGVTQLPESIVQLKRLMHLVGQRLIMPDGFGSMESLEELGTIDCCKCPVSFGEDLALLSRLRVLRVAFIGVETSDMETRRKSLMSSLCKLGGDNLRRVTIIDLAGGGDCFVESWHPPPRLLQKFIHISQQQHFSRFPEWISSCLCDLTHLDIKAEKMEREHLSVLEHLPAIRCLYLFVKRVSEDGLAISHGAFRCLRRLEFCNVDGPGLMFAGGVPMLEWLRLGFDADRAQSTYGGLEVGIQRLSSLKHVVLIVWMVSEGGDDPAEQAVWSAINGQVEMLPNSPTVDIRFRRRSQLQASSE

SEQ ID NO: 9 - DNA, 11437 bp, Zea Mays
  source 1..11437: mol_type = other DNA
ccgagtacattgccgcaggacattgttgcgcgcaattgctttggatgaggcaaaccctgcgggactacggttacaaattaaccaaagtccctttgctatgtgataatgagagtgcaattaagatggccgacaatcccgtcgagcatagccgcactaagcacatagccattcggtatcattttcttagggatcaccaacaaaagggggatatcgagatttcttacattaatactaaagatcaattagccgatatctttaccaagcctcttgatgaacaaacttttaccaaacttaggcatgagctcaatattcttgattctagaaatttcttttgctaagcttgcacacatagctcatttgaatacctttgatcatatctcttttatatgctatgactaatgtgttttcaagtctatttcaaaccaagtcataggtatattgaaagggaattggagtcttcggcgaagacaaaggcttccactccgtaactcatacttcgccaccactccgagcaactctctcttctttgggggagaaatgagcatcaaggaaaaggacttcatccttgggggagagaataaaagctcaaacgcaaaaggacttcgtctttggtataatcttaactcacttatttatgaccaaaggggaagaacttacttctagggctctaatgattccgtttttttggcgattcatgccaaaaagggggagaaatgagcccaaagcaaaaggaccgcaccaccaccaccaaattcaaaaacttagtgctttccaaaagtctttatcatttggtatcctattgtgttcaaaagggggagaaagtagtatttcaaaaatggtatatcaaaaccctcttgaacactaagaggtgaatctcttttagggggagttttgtttagtcaaaggaaaagcatttgaaacagggggaggaaatttcaaatcttaaaaaatgcttttgcaaactcttatttatttacctttgactatctgcaaaagatctatgaaaaggatttacaaaaggttttgcaaaaacaaaacaagtggtgcaaacgtggtccaaaatgttatataagaaagaaacaattcatgcatattttgcaagtatttatattggcccaatttcaagcaacctttgcacttatattatgcaaactagttcaattatgcacttccatatttgctttggtttgtgttggcatcaatcaccaaaaagggggagattgaaagggaattaggcttacacctagttcctaaatagttttggtggttgaatcgcccaacacaaataaattgaactaactagtttgctctagattacatgttctacaggtgccaaaggttcatctacaactatactaattcgactgtccggaataccgtagattattccggacaggagaagctttttggaaaaacaggccaagcgcggaccgtccgggctcttgcggcggaccgtccgcaacatcgggatactcctcggacagaaccaatgcaaaaacacaagtttccactacagactgtccggaggaaaagcaaagaccgtccgagcccttgcgcggaccgtccggcctctggcgcggaccgtccggtaggtgagaaaccgaaaaacccgaaggtaacgggttcggagaaatgaattatagcggcctcgcggaccgtccggaccaggcgggcggaccgtccgcgactggctctgtctgacatctgacgacgcattaaatgcaatatagccgttgatatagccgttactgctgaccgttgcattttcagccgttgatctacaggggcggaccgtccgcaccaggagggcggaccgtccgcgctcagcagaatggcccaacggctaggaagtggttggtggctataaatacaaccccaaccacctccattcaatccatccaagcattccaaccttcaacattcaatacaagagctagcaatccattccaagacacattcaaagcctccatctctccaagtttcacaattgagaaaagagatcattagtgattagtagcttgagagagaaagtgatccgtgtgttatttgtcgctcttgttgcttggctttttaatcgtgctttcttgattctttcattgcgatcaaactcacttgtaattgaggcaagagacaccaatcttgtggtgatccttgtaggaactttgtgttccaagtgattgagaaaagaaagctcactcgatccgtggatcgtttgagagagggaagggttgaaagagacccggcctttgtggcctcctcaacggggagtaggtttgcgagaaccgaacctcggtaaaacaaatccgcgtgtctcacttacttattcgcttgcgatttgttttgcaccctctctcgcggactcatttattattactaacgctaacctcggcttgtagttgtgattatttttgtaaatttcagtttcgccctattcacccccccctctaggcgactttcactcaccagtattgatttaagaactaagcgccttgttattactggtctatttggataagtaggaatgacttagtttttaataatatttcaagttttacttatctgcaggttttttttccagaggtacacactggcttagattctgggctcaactacaaaaggacgaagctgatggagttttaataaaggatgtttgccgtcgccgggaatcggtggtcatgcaattttgtgttaattttggttggaggttttctaataggattcccttttaatcatcttggtggttaaaaaaaaatcaaaaacctttagtgtgctatgtcccttcagagagcagtgtaatataacattttgtgttgtctctaatctattttgaggagaaagccggaactttttctcgttatctgaaaaaatctttggctagccttcgcgactcgccatctcccagccgtggcgctctctcgcccacgcacaccatgcagctgcttcgtcatttccagccatatgcgtgaagacatttgaattcgtccaccactgcatcactagtctcactctcacctataatatatctgcacccaacgagcaggtaccaagcaattagcaagagaaacacaaggtcgaggtggtgaggcatggagctcgccttgggggccatgaccagcttggcccctaagcttggcgacctgctcatggagaagtatgtcgtgcagaagggcctcaagcccgacatcgagtctctctccagggagcttgtgatgatgaacgccgctctcgtcgacgcgtcccgggttccacctgaccagctcaccgaggtggaaaagctctgggcacgcaaggtccgggacttgtcgtatgacatggaggacgccgtcgacgatttcatcctgcgtgtggctggtggtgacgactctgccgccgactccaaattcttcaagaagacccttgccatggtcaaggacgtgatgtcgatgaagaagttcaaggatcggtgccagatctccgacaaggtcaaagacatcaagaaactctccaacgagttagctgaacttcgtgccaagtacacggtaaggggtgtgggtgctgatctcgccgcgagcaccggcatcgacacccgtgtcatcaatctgtacaagaaagagacagatctcgttggtatcgaggagtcaagggacaaagtcattaggatgctgtctataggggccaaagatgaagatgcacatgagttccatcaggatctaaagatagtgtctatagtcggggttggaggactaggtaagactactctagccaaaacagtgcatgacatgcttaagaagcaattcgactgttgtgcttttatttctattggtagaactcctaatctgaataggacattcgagaagatgctattggaactcgatcgtgagtataaacaagttgacatggccagatgggatctagaacaatttataaacgaactggatgaattcttgaaggacaagaggtacgcccatgtcattcattctccctctagtttagttgaattttttcattcactagtagagcgtgggcagttttgccatgtttatatgcataccgatttgccaagttattcatagctgtacactcttgtgtcaattttatatatatatatatatataggtacttgatcgttgttgatgacatatgggatgttgactcatgggaagcgatcaaatatgccttaaaggacaatagttgtggaagtagaataatcatgactactcgcaattctgggtttgtcaagaaagtagaagaggtttatagattaaaacctctttctaatgaaaactccaagaaactgttctacaaaagaatagagagtcaggaaggagaaagccttgatggtgaactctctagtaaaatcatacataaatgtggcggcataccattggctatcattgcaatagctagtttgttggttgaaagatcaagtgaggagtggtcagaagtgtacgacaagattggtcttgggaatgaggacaatacaacaaagataatgttatacagctactatgatctgcctccttatctcaagccatgtctgctgcaactaagcatatatccagaagactgtttcattgatacaaaagctaccatatggaagtggataggtgaaggtctagttcatattgagaaagaggagggtagcctatttaaggttggagaaagatacttcaaggagcttgtgaatagaagcatgatccagccgatagagaacataaatgattggtttgtagaagagttccgtattcacgacattgtgtttgatctcatctgtaagttgtccaaggatgaagacttcattagccttagcgggcaacattcatctcaggatagtttaagaagagagaagaaaacaggtgtgcctcgctcagactgcaagctacgtcgtctggtcgtccgaaatcaacgtgtgcagcgcttccctgaagaaaccatggacatgccagaggtgttgagatcacttagcattatagattgtaatattgcggttgtggccccaattgatagcttcagggtttgccgtgtgctgtctatagtaaacaactacgtacccatcagcctaaagcatctggggaagctgttgcatctcaagttcctagagatagtatacacgcctattgatgagctccctaaggaaattgggcatctgaggtctcttcagacactgatattagtccgtactggactagacgagctgccaccggctctttgctcgcttacacggctcatgtgtctgatagcctatggcttcgaaaggttgccagctgataggatggggaacctaacgtccctggaggagctacaactaaatagggtagttggccggagtgccacccaagacctagtggcagagtttggcaagctgacgaggttgagggtggtcagcatcaccttttcagagcagctagaggagagcttgcaagaagcattggtgcaatctctgtccaatctgcggcgactccaggaactagagcttttgtgtaaaatgccagagcggggaagcgatatgtggggagactgggagccaccaaggcagctccggcgcctgattattgaaggcatcgacttctcacggcagcctcgatggatcaaccgctcctgcctgccacgcctctgctccttatatctgagggtgcacgctcttgaagcacaggacctagataatctagcgaggttgccagagctccagtacctccagctatttggtctcagctttcctccaaggtatactgttggcccagacgacttcaggaatctgaggttctgcgaagtgggcacaacgttcgagtttcgtaagggcgccatgccaaggcttgaagtgctgcgatttggagtttatgcagggtactggagttgggaagagaatggtgtgccgttcgagcagttcccaacgaaggatgtgatcgaagatcttcacttggacctggataacgtccttttacttcagcaagtaatagtcaaagtcaactgcttaggtgctactgccgcacaagtggaggaggtggaggccgtggtcatgcgtgccgtggaaaatcatgccaaccgtccaaccataaaaatggatcgagtatatgaagaaaatatcttatctgatgaaaagtgggaggctctggtgagtctgctaaagaaaatatgttttcttatatatatttatatatatatatatatatatatttatgtttgtgcacttcaattctcagcttcggcgacacattgaagaggattgctgcgtgcgcacgatgaaggataaatctaatgctttcttcatcagccagctgtggttatatcgacatcttcaggaagccattattttcatcgactgttcgggtgccagcatgtgtgaggtgcagaaagtggaagcagcttatagacatgcagccgaggttcatcctaaccatccaagtattgaacttatcagaacaaacaccgacggaatggcctcctcctcatctgaccatcccaacacagaggttcgtcctagctgcgtggaaaatttttcagtaattaatggaaaccttagctttgacggtataacttaccaccaacgcttatttttcgttcccaaactccagcccaggaattgatgactcggtttgtcagatccttcgacccagcaccagcagcaggtacgtgtgcatgcccatgatctacactcttaaattaattttattactctatccagttatcacgttccaatgccatgtgtaagttttagtacctgtttatttttcgcttgcaattgttgcatcttgaactcttgcttctaacctccattatttgtctgtccacttcaccaagagacagaaaatcatagctctaacttccatgaagcctaaacctccaaactccttgggtctgtttagggcctcccaccttgtcatgtgatacttccttttcttgctagttccataaaaagagatctgattgagtctaacatctagaaattctcctcgtacaagtgatagacccccactgtgttcataggaatgcttgacaggcaggattccattacagtagatatctttccagaagatagatgtccagactgccaagtacccaacctcttctgagtcttgtcactgaatttcatctgtgctttagtcactcataggaatcttcaaatatttgataggaaaaatttccaccttgcaacgaagaagtttagctatcctctctttctcctgatcctcagccccaacaacagaaacttaacacttctcaaagttgattttcatccttgacattgcatcataacaagataaaataactctaatattgttctggttaccttttgagttctgtaaaaaatggttgtatcatcatcatattgcaagtaactctgaaattaagccctttagcggacaacctcttctagctctaataaggcagataaagcatctgtcactaagttgaacaaaaaagggaaaagaggatcccctgtctgagacctttaaaacttctaaaatagtcacctctctcaccactcaaatcaataggtatcatgtcccactgaactgcctgtctggtccaacctgtccatatattagaaaaaaaacattttctttgaagtacctcctccaaaaagttccaattcaccttatcatatgccctctaaaaaatcaagccttaaaataattccaaaaacctttatagctcttagctcatggagtacatcttgcaaaagttgtctggtatttgcttatcactttatctgctgcaactgttaacctcagattaagcatttcagtaaggatcttatagatcacattcagtaaacaaataggcctatattgtctagtagaggtggctggcttgctcttaggaaataggataataactccatagttctagcttgcctgcattgcatagcatttccttaacgtcaccacctatcagatgccacaaaggcttaaatttttttactgtaaatccatcaggaccaggagcggtgttattcttcatgtagtttctttcttttcttttcttttgaaacatagtttccaaatattttttaagctactttggttctcatttcagttagtagacgtcaaggtttcatacaaaagctgatttatagattacttgcgttgaatagctaaccaactaaaatgcaccacgtatgctaatcaaaccgatttccttatggtaccctgataaaaagcgtgctgtttttttagaaaataaatgttaacatgttaagtggtttgataggtcctaagatgattttaagtgaaaggaactaatcaaagcagcaaaagaaagaaatatatcatatacctggatggtagctcatgttttaatcttgtaaaagtgcttttactaaactgaatcgttttggattagacatgtttattggctgttgagtttgtgatccaaattctgaagaaggcggccaagttggcgagcgcggatcgttttagggtttcacctctataaatatctttgtaagccgcaggagatatctatctcaattgtaaatctcttgcgatctgtaaccacccgaaaatagtgagaagttgcctgccggcgcccgtggttttttccccttcactttggaggggttttccacgttaaatccgtgtcttctctgtgattgatcctatttgtgtcgcattcatttataacattggccaaaaaaaaccttgtatcattactgtttgcttgaagaaattctgtgattgcatttttcctttacatgtttgctccatcctttgcatttcaacttatttactatatacacaaaccaatatttcaggtaaatgtgagttgcttcagtaccaaagagagaggtcctgttcagcaagatgattagaagcattggcatggcttgttaattatatttgtggttcttagtctaaaatcacattgaagagggagatggacccaagtgatgcagttcatgtgttacctgtatctgtctctattttataagctgtcatatattttctcgaaactatcttgtgtggattttgtgtgaagtgtacttttagtggcacaggccatgtatttccttggaaacgaccatgagagtttgtatatggacttattttccccccattttctttcctgagatgggctgtgtatttgcttgaaagatagggttggtattatctttgtatttgaactctaaatgcttaaatgaagtgatcatcctggatggagatatgtatggtgtgaaccctattctcgatgccaacgtgtgtgggattttggtgcgagctcctgtagctcgtctttgttatatcgatggtgttcacaatatatgcaggttcatctaacacgaatgatgaaactatatgcaggcggtgccggtcagcagagggttgcctgacgcggagaagagatgcaggctttgctggctctggaggaattcgaacgtgtggcacacacagatgtgaagcaaagatacaatacaacaaggtcttccgtagccagtagtagccagtagggaagaagaagaaaaacgacccgagtttctcaaggttgtcaagctcaaaagttagaaaaagggtcaacggagctgattcccttcacaaaagttacgtcagcaataaattaccccgttcattgcgaatggggttagttaaaaaaagggtcaacaaccaaaattagcgtgcaagaggagaaaactatgtagtataaaatgcgcatgagatcagaagccacattaatgcttctgttgttacctaacaatcagggaggaaatttctggaagttcaactggtaggaggtgtgcatccgaaagataaagagcacagaacaaacgatcataatttagacgaataagataacaacaaagcttttgcaacatagtatttggacaaggggttgaggcaaggagacccgctctctcccattctttttaatgtgatagtggatatgcttgctattctgattaatagagctaaagcagatggtcaatttgacggtctagtgccacatttagtagatggagggctctctattttgcaatatgcagatgacactttgttgtttttttataatgatttagagaaagcagcaaatctgaaatcgcttcttgtagcctttgagcaagtatccagtttgaaaattaattaccataaaagtgagctattctggcaaggtgaaaatcataagaagaaatacatgcttgctatatggaatcctttgccaacctaaggatcaagggggttaggtattaacaacttagatttacaaaataaatgcttgctaagtaattggtttttcaagctagttaatgaggatggactttggtaaagtatgttgtggaataaataccttaagagacacaccttgtcaaaagtatccttcaggcggtgattctcatttctgatcgggaattatgaaagtgaaacatttgtttttttaacctaggatctttcattttaggtaatagagaacaaataagattttgggaggacgtgtggttgggagaccaaccactaatgaggcaatatccttcactatatcatattgtgagacagaaaagtaatattgttgcgtctatcttgagtacgacccctcaatgtttcgtttcgtaggacgttgtgggtcagaacataactttgtggtatgatcttgttaatcgggtggtgctcacctcgatgagctataatagagatgtctttaagtggagggtaacatcttcacggtgcaatcaatgtatcatattctaattaataacggtcaaatgtttaatcacaagctgatttggaaactaaatttaccctaaagattaaaatctttttgtggcatttggttaaatggatgttctaactaaggataacctaaccaaaacaaattcgaatgggaacaaaaaatgtgggttttgtaatacacatgagtctatttaacatttgtgtttagaatgtaattttgctcgccatatgtggaggttgtttcacttctgttttggtatgagtgtaccgaggtctgttcgtcatatttttagtacgtggctcaccgctattgatttaagaactaagcgtcttgttattacatgtgtctcagtgttttgctggactatttggataagtaggaatgacttagtttttaataatgtttcaagttttacttatctgcagggtttagattctgggctcagccctcagctacaaaaagttgaagctgatggagttttaataaaaaatgtttgtcgtcgcctggaataggtggtcatgtaattttgtgttaattttggttagaggttttctaatagtattactttgtaataatctcggtggttttaaaaaaaatcaaaatattttagtgtgatggtgtgtgactgtcccctcatagatcagtgtaatataacaatttgctaaccttttttaggagaaagcagtaactttttctccattatctaaaaaaattctttggctagccttcgcgagtcgccgtctcccagccgcacgctctctcgcccacgcacacacgattccttctcgcgctctcg

SEQ ID NO: 10 - DNA, 3129 bp, Zea Mays
  source 1..3129: mol_type = other DNA
atggagctcgccttgggggccatgaccagcttggcccctaagcttggcgacctgctcatggagaagtatgtcgtgcagaagggcctcaagcccgacatcgagtctctctccagggagcttgtgatgatgaacgccgctctcgtcgacgcgtcccgggttccacctgaccagctcaccgaggtggaaaagctctgggcacgcaaggtccgggacttgtcgtatgacatggaggacgccgtcgacgatttcatcctgcgtgtggctggtggtgacgactctgccgccgactccaaattcttcaagaagacccttgccatggtcaaggacgtgatgtcgatgaagaagttcaaggatcggtgccagatctccgacaaggtcaaagacatcaagaaactctccaacgagttagctgaacttcgtgccaagtacacggtaaggggtgtgggtgctgatctcgccgcgagcaccggcatcgacacccgtgtcatcaatctgtacaagaaagagacagatctcgttggtatcgaggagtcaagggacaaagtcattaggatgctgtctataggggccaaagatgaagatgcacatgagttccatcaggatctaaagatagtgtctatagtcggggttggaggactaggtaagactactctagccaaaacagtgcatgacatgcttaagaagcaattcgactgttgtgcttttatttctattggtagaactcctaatctgaataggacattcgagaagatgctattggaactcgatcgtgagtataaacaagttgacatggccagatgggatctagaacaatttataaacgaactggatgaattcttgaaggacaagaggtacttgatcgttgttgatgacatatgggatgttgactcatgggaagcgatcaaatatgccttaaaggacaatagttgtggaagtagaataatcatgactactcgcaattctgggtttgtcaagaaagtagaagaggtttatagattaaaacctctttctaatgaaaactccaagaaactgttctacaaaagaatagagagtcaggaaggagaaagccttgatggtgaactctctagtaaaatcatacataaatgtggcggcataccattggctatcattgcaatagctagtttgttggttgaaagatcaagtgaggagtggtcagaagtgtacgacaagattggtcttgggaatgaggacaatacaacaaagataatgttatacagctactatgatctgcctccttatctcaagccatgtctgctgcaactaagcatatatccagaagactgtttcattgatacaaaagctaccatatggaagtggataggtgaaggtctagttcatattgagaaagaggagggtagcctatttaaggttggagaaagatacttcaaggagcttgtgaatagaagcatgatccagccgatagagaacataaatgattggtttgtagaagagttccgtattcacgacattgtgtttgatctcatctgtaagttgtccaaggatgaagacttcattagccttagcgggcaacattcatctcaggatagtttaagaagagagaagaaaacaggtgtgcctcgctcagactgcaagctacgtcgtctggtcgtccgaaatcaacgtgtgcagcgcttccctgaagaaaccatggacatgccagaggtgttgagatcacttagcattatagattgtaatattgcggttgtggccccaattgatagcttcagggtttgccgtgtgctgtctatagtaaacaactacgtacccatcagcctaaagcatctggggaagctgttgcatctcaagttcctagagatagtatacacgcctattgatgagctccctaaggaaattgggcatctgaggtctcttcagacactgatattagtccgtactggactagacgagctgccaccggctctttgctcgcttacacggctcatgtgtctgatagcctatggcttcgaaaggttgccagctgataggatggggaacctaacgtccctggaggagctacaactaaatagggtagttggccggagtgccacccaagacctagtggcagagtttggcaagctgacgaggttgagggtggtcagcatcaccttttcagagcagctagaggagagcttgcaagaagcattggtgcaatctctgtccaatctgcggcgactccaggaactagagcttttgtgtaaaatgccagagcggggaagcgatatgtggggagactgggagccaccaaggcagctccggcgcctgattattgaaggcatcgacttctcacggcagcctcgatggatcaaccgctcctgcctgccacgcctctgctccttatatctgagggtgcacgctcttgaagcacaggacctagataatctagcgaggttgccagagctccagtacctccagctatttggtctcagctttcctccaaggtatactgttggcccagacgacttcaggaatctgaggttctgcgaagtgggcacaacgttcgagtttcgtaagggcgccatgccaaggcttgaagtgctgcgatttggagtttatgcagggtactggagttgggaagagaatggtgtgccgttcgagcagttcccaacgaaggatgtgatcgaagatcttcacttggacctggataacgtccttttacttcagcaagtaatagtcaaagtcaactgcttaggtgctactgccgcacaagtggaggaggtggaggccgtggtcatgcgtgccgtggaaaatcatgccaaccgtccaaccataaaaatggatcgagtatatgaagaaaatatcttatctgatgaaaagtgggaggctctgcttcggcgacacattgaagaggattgctgcgtgcgcacgatgaaggataaatctaatgctttcttcatcagccagctgtggttatatcgacatcttcaggaagccattattttcatcgactgttcgggtgccagcatgtgtgaggtgcagaaagtggaagcagcttatagacatgcagccgaggttcatcctaaccatccaagtattgaacttatcagaacaaacaccgacggaatggcctcctcctcatctgaccatcccaacacagagcccaggaattga

SEQ ID NO: 11 - protein, 1042 aa, Zea Mays
MELALGAMTSLAPKLGDLLMEKYVVQKGLKPDIESLSRELVMMNAALVDASRVPPDQLTEVEKLWARKVRDLSYDMEDAVDDFILRVAGGDDSAADSKFFKKTLAMVKDVMSMKKFKDRCQISDKVKDIKKLSNELAELRAKYTVRGVGADLAASTGIDTRVINLYKKETDLVGIEESRDKVIRMLSIGAKDEDAHEFHQDLKIVSIVGVGGLGKTTLAKTVHDMLKKQFDCCAFISIGRTPNLNRTFEKMLLELDREYKQVDMARWDLEQFINELDEFLKDKRYLIVVDDIWDVDSWEAIKYALKDNSCGSRIIMTTRNSGFVKKVEEVYRLKPLSNENSKKLFYKRIESQEGESLDGELSSKIIHKCGGIPLAIIAIASLLVERSSEEWSEVYDKIGLGNEDNTTKIMLYSYYDLPPYLKPCLLQLSIYPEDCFIDTKATIWKWIGEGLVHIEKEEGSLFKVGERYFKELVNRSMIQPIENINDWFVEEFRIHDIVFDLICKLSKDEDFISLSGQHSSQDSLRREKKTGVPRSDCKLRRLVVRNQRVQRFPEETMDMPEVLRSLSIIDCNIAVVAPIDSFRVCRVLSIVNNYVPISLKHLGKLLHLKFLEIVYTPIDELPKEIGHLRSLQTLILVRTGLDELPPALCSLTRLMCLIAYGFERLPADRMGNLTSLEELQLNRVVGRSATQDLVAEFGKLTRLRVVSITFSEQLEESLQEALVQSLSNLRRLQELELLCKMPERGSDMWGDWEPPRQLRRLIIEGIDFSRQPRWINRSCLPRLCSLYLRVHALEAQDLDNLARLPELQYLQLFGLSFPPRYTVGPDDFRNLRFCEVGTTFEFRKGAMPRLEVLRFGVYAGYWSWEENGVPFEQFPTKDVIEDLHLDLDNVLLLQQVIVKVNCLGATAAQVEEVEAVVMRAVENHANRPTIKMDRVYEENILSDEKWEALLRRHIEEDCCVRTMKDKSNAFFISQLWLYRHLQEAIIFIDCSGASMCEVQKVEAAYRHAAEVHPNHPSIELIRTNTDGMASSSSDHPNTEPRN

SEQ ID NO: 12 - DNA, 23 bp, Zea Mays
  source 1..23: mol_type = other DNA
gcacgctccaggttaatggctgg

SEQ ID NO: 13 - DNA, 23 bp, Zea Mays
  source 1..23: mol_type = other DNA
gcagctgaaattgagcctcccgg

SEQ ID NO: 14 - DNA, 23 bp, Zea Mays
  source 1..23: mol_type = other DNA
gattagtctcggcatacgtacgg

SEQ ID NO: 15 - DNA, 23 bp, Zea Mays
  source 1..23: mol_type = other DNA
ggataatggcgtacgtattgcgg

SEQ ID NO: 16 - DNA, 23 bp, Zea Mays
  source 1..23: mol_type = other DNA
gtttcgaacagaacgtacgcagg

SEQ ID NO: 17 - DNA, 23 bp, Zea Mays
  source 1..23: mol_type = other DNA
ggctaggcgtgtcaccataatgg

SEQ ID NO: 18 - DNA, 23 bp, Zea Mays
  source 1..23: mol_type = other DNA
gaatacgaaactataccgcgggg

SEQ ID NO: 19 - DNA, 23 bp, Zea Mays
  source 1..23: mol_type = other DNA
gactacctctgggggtacgtagg

SEQ ID NO: 20 - DNA, 23 bp, Zea Mays
  source 1..23: mol_type = other DNA
gacggggacttaattatgcgtgg

SEQ ID NO: 21 - DNA, 23 bp, Zea Mays
  source 1..23: mol_type = other DNA
gcgatccgtcacttgtatatcgg

SEQ ID NO: 22 - DNA, 50 bp, Zea Mays
  source 1..50: mol_type = other DNA
aaccagcagccaaaggacaagacaagagacacaacgaaaggaaaggaacc

SEQ ID NO: 23 - DNA, 50 bp, Zea Mays
  source 1..50: mol_type = other DNA
caactagtgggccagatatcaccagccggaaattagcaaaaatgctacgg

SEQ ID NO: 24 - DNA, 12662 bp, Zea Mays
  source 1..12662: mol_type = other DNA
atttaatgagacctgtccattccgcaggcggcgtgcctgtccattccactggcaggcgatgtgctacctccgcatttaatgagacctgtccattccgctggcaggcagcgacctgtccatcccacaggcggcatgcatgtccgttccattggcagactgcatgcccataccgccgcgtgcactacgcccatcattactcgtatgttaccaaggaagctgccactgcatgtcaacactgcgcgtactgcggacaacatgggcctggagattgcacagacgtcacctgcattagttgctctaggtattccattcattatgttcctgggcccacatgttggggctcagcacccttgtatgtgcctcccttgagctataaaagggaaggcacacaacgttacaaggcaagctctcaagtcactcagacttacttagaccctcgagaagttctccaagctctcgagtatcagcaatactacatagtggagaagggttttacgctccggtggcctgaaccactctaaactcttgtgtgctctcgtgctttcatcgaccatctagcagacaggcaaaacgcttaggccccctcctcatcttaggatttagggcgggtgcgttccgccacccggctagagatttcctctccgacactcacatacttccacgtcttgctcacaaatgctcgacaagaccattttgtagtgtgcaagacctttcctagcgacgaagattgaagtgattaatcacccaatccgagtgtccttcaatgagttggcacggttcaaatgcagaactactctctccgaaattggtatttgctgcaaagtcgccttcgtcttcgacaatcatgttatgcattattatgcatgcagttattatctcagtaagacagttgcaatcccaaccgtacgcaggatcacgtagcaccacccagcgtgcttgaagaactccaaatgggcactcaatatccttcctgtaactttcctacatttgtctgaaatatatctttttttcctcataagggtgtctaatggctttcacaaaagttggccagttcagatatatgccattagctaggtaataaccaaagttgtatgcatgatcgttgattatgtaatgaatgggtggcattcgaccgcttgtcatagggtcaaataccggcgatcaatgtagcacgttcacgtcattgtttgttctaggcatgccaaaaaaaagtatgccaaatctagagatcgtacgttgcgacagcctcgagtatcatggtaggtcttaaatttcgaccacagaactatcctcgccacgcggttggacagttcctccactcccaatgcatgcaatctatggaacccaacattcctggaaaccccctcgactcgctattgtgcatgatgcgtgcaatgtctgcttcattaggagttcgtagataccacccactaaaatatgcaattaatgcacgatagaaatgaataaggcattccctaacggtagactcccctatttgaatgtactcgtctacgacatcagtaggtaagatgtaagcaaggatccgcattgctgcacatactttttgaagtggttcgagtccagctaggccagtagcgtcgacccgttgagtaaagtaactatcctgctgttgcagtccttccataatgcggaggaacaatgaactactcatgcggaacctataaaatgtaataggtaaagtatgaaagacactgttaatgcaaaacttaataatacaagtatgtagttgttaccttctgcgaaacacgtggggtggatacacgagatttagccgaagtagtgatggtggatgagattctctccagcataatgatccctgtgaatcacgcggcgggggataccggaacgtctacacctacggtgtgtagccttaatggtgagggtaagaagagtggcaagcataagtgtgttgtcgtcactatcactcgaatcggaatcatcgtccctacgcaaagacgacacaacgtacaaagtcttgttagcttgagttgaggagtgagatgtgcacacaaatgggaagagcacgtacccatatgtatagacgagatggagttagcttgtgttgcaagctcgtgtcttgcactgaaagcctcatgtgtcttgctgtgtatggttgtcgcatgggtgcttgcaatggaatttggtcgaatattcatgtcatatttcaatcgtgctgaaatattgggtgccaacagattcataatgcattggaagggaggcttgcagtgtacggttgttgcgtatgtgcttgcgaaggaaaagcttcgaacgataatgcagatcaactatgagcatgggtatggtccaataacatcgatggtaattacaaaacgaaaaataattacttcgttgttggtaatggtatatgatgaatacgaatttaataaatattcaccagaaatatgtacgacacaatatatttaattatgagttttttaaaatttagaaattaaataataggtaattatacgtattatgtgtcccacaataacttatagagattatatgtctaattgaaaaattaattcaattattcaatggatttatacgttaagaaaataaaaattgaaaaaatatggtagttaatgatagttggattgatatatatatattttatgcgtagaggatatataaaggaatatatagacggaatagttacagaaagattgaatatagtggagtgaattttgctgtgctacgtagtaatatataacgcgaaaaatttagggaaacgctgagggctgcagatacagctggccatgctatcaatagattagtacttggttgccacttgccagtagccgcactttcctcgatatgcccaagcgagagagagagattcctaagttgattcttctgtggatgaagatgaaatctacttctgaaatcttgccaaccgtccaactctgtttgatctaatttctatatcctccggccaccaagttcgtgtcaaataagcgagcacagctaggtaagtagctgattcgaattccaacaaaacatctcatcccttttagtctcttgtgctagatcttatgcgtctgcaaccttgggagctagctagccatggagttcgcaactggagcgctaggcacccttcttcccaagctctccatgcccaagcgagcgagagagagagagattccttagttgattcttctgtggatgaagatgaaatctacttctgaaatcttgccaccgtccaactctgtttgatctaatctctatatcctccggccaccaagttcgtgtcaaataagcgagcacagctaggtaagcagctgattcgaattccaacaaaacattgtatcccttttaggccgtgtttgttttggcttctggcagcttctggtcattaaaagctgctgcaaactgtcaaacgcttagcttttcagccagtttctataaaattcgttgaggcaaaaatcatccaaaatcaacataaacacataaccggttgagtcgttgtaataataggaatccgtcactttctagatcctgagtcatatgaacaactttatcttcgttcacacgtaatcgtattgatactcagcttctcactacagacagattctcctcataatcagattttcaaaaaaactgaatagaaaaaagctaaaccaaacgtgccttagtctgttgcgctagatctcatgcgtctgcaaccttgggagctagctagccatggagttcgcaactggagcgctaggcacccttcttctcaagctctccatgctgcttcatggtgagtacaacctggagaagggcgtcaggggggacatccagcgcgtcatgaacaagctcgagcgggttcattctgtcctcggccatgttgtcgaagtgcctgtgccactgaagccacgtcctgatctggtcagaatgttggcacgcagcgtcagggagctatcctacgacatggaggacttcgtcgacaccttcctggtgcgtgtccaaggccctgaacgcaccagcaaaagaagcgccaaaatattcatgaataagacatatatggtcgtgaatcgccatgagatcgcccagaccatcaaggacttcgaagagcgcgtccagcagatagatgagcgtcgtcaaaggtcattgaaacacttaatttccactttggcttgcgtgcatatgaactttcacctttactgtctttcttccattttatttccttttctggttctaacaggtacgatgttgatgctatggttccccgtgtcaaaaccttggttgatcctcgcatatttgctctgaagtacaccaaggccacggaccttgtcggcatggatgaggcaagggaggaactaatcacaaggttgaccaaggaagatgacacctccactgaacaaaggcgagtctctatcgttggttttggaggacttggcaagacagcgcttgcaaaagcagtttatgacaaacttaaagctaaaggggaattccattgcgcggcctttgtgtcggtgtctcggtttcctcagctcgaaaaattcttcaaggaattgctttatgagcttgacgagactgagtacaaggaacttattgacatcagcaccccattggaacttatgaatctagtgcatgaattccttcataataagaggtacacgcgtacatgtaccacacctagtgtgcattatatgttcattccacggaaatatccatattatagagtaatatgcttatatatattattgcaattcatatctttagctatgcttacattacttctccaattaatacaataaatataggtaccttattgctgtcgatgacatatgggatactgacgcatgggcaatgatacaatgggcttttcctgagaataagctaggaagcagaataatcgcaactactcgcagaattgatgttgctgagtatgtaggtggttgctatatgatgaaacctcttactcgagagaaatcaaatatattattctatggacgaatatttggctctgaaggtaaatgtcctcctgaactttctgatgcgtctgagaaaatattgaataaatgcggaggcgtgccattggctattattactacatctagcttgctggctagtaagtcaagaaacataaaagaatggtacaatgttgctgattctattggttccagaatactaaaaaacagtactgaaatggagaatttgaggaacatactgctgcttagctattatgatctaccagcacgattaaagacatgtttgttatatctgagtattttccctgaagattgtgagattgggatgcatcggttaatatggaggtggatagctgaaggttttttcaatggagaactagcacatgatgggctctttaaccttggcgaatcttgtttccacgagctcataagaagaagcatggtgcagccagtaacacttgaaggcacgggtcttgtatatgcttgtcgtgttcataatatgtttcatgatttgatcctctacatgtcacacgaagaagaatttgtttctgcagtcaatgaaaagtttggtcttctagatgttcgttctcggcggttagcattccagaacataacaaaagagcagtacagacttgtggaacatccacggctggcacaattgaggtcacttaatgccattggatgtcctatatacgcgatacctccaattgaaagctataaattattgcgtgtactggatttcgaaaattgtgcaggtattgaaggccatgatcttgttcatcttgggaaattgcatcacctcaagttccttgggctaagaaacacgtttatcggtaagctgccggaaggaatagggaacctcaagtttctgcaaacattggacctcgatggaactggtgtggaagaattacctcaagccttgcataatcttacagaattgatgtgcctaattgctgactggagaacgagagtgcccaattggattggtaacctcacgtccctgcagcacttggtgatttatcctggtgggcatgacgatgaggattctgcgagcaggtttgttaatgagctgggaaagctgagacaactaagggtgctccgttttttgataaaagcacaagatgaagggcagctgagagatttgctagagtccctatcgaatctgccagagatcgaggctatacattttgattactatggagtacagttaaatagaggtgttcagttggaacctgaaggctatgccctctctagacatattcgttccatggaattgcgctggttggagttctcaaggctgcctctttggattaatcctggacaacttcctaacctctaccacttatggctgatggtatctgatgcggaagagcgggatctggaaatccttggggggtttccagtgcttcactccctccacttgttgattgtgaatactgaacgtgaagatgtcatgacttgtggctgtggtggattcaagaatttgaaatgctgcagtataactaaaccgctgaaatttgtacatggagctatgcccaggcttgaagtcctcgatttccatttcagtgtgcaactcctaacggattcaaaccaagaatttgattttgactttggcttgggaaacctacattggcttcagcaagccatcgttcaaatcacagcccttggtgaggaggtggagtctgtggggagagcacaggtggctctgcgggatgcaatacgtacccatcccaaccgtcctacccttgaaataaacttatttgggcaaacaatacctccagagttaccaaaggtcaggactctttgtcatctctctctcacctcatttcgttgtcttgttttcatttgccttatttcactgtttcagcaagacgacgatggagcgaaaattgtggagatatcaccggccgagagtagtcgtcaagctcaagagcgggagaaaagaagcatcgatgtggcgacgaagaaagcaacacgggtgccgtctttttacacaaagtcatcaattgatgagccaatggatcagctcataaacatgctatctgtggttgatgacgaagcctacactaagaacataaagatactatctattgtaaggtctgagggactggggaagactactctggcccaaaaagcattcgaagagctccattcgcaatttgaccgtggggcgttcgttctactaggccagaatcctgacttgaggagagtctttgctgacattctccgtggtcttgacaagcaaaggtacatagatttcccagtggcaatattggatctagtggacctgatctggctagtccgtaaatcgctcataaacaaaaggtatgtccaacccactgacatgctagtagcctagtactacctttattcagctatatatttgctcaatatattgtctccgttctttttttttatttgacgtggttgattattttttcttcaaaattctaatcactcgtgtactaaaaaaaatctgtgtgttatatccatgagtgttatttcgacactgccggttaaaccggtgagtattgatttctctgattttgctttgtttgacttggttcttcttggtgtcttcagcatttcatcccatagacctataaacctgagaatatacatctagaaaacattgttaatctaagtgttgtgtgtcaatcaatcaccaaaacgaagcaccgaaatatgacataagagaccatttttgctacagttgttgtcagtagtagttactactaaatatggattataacccttggatactattatttttccaggttctttattgtatttgatgatatatgtgatgtaaaagcatgggaaattataaagtgcgctttgattgaaaataacaaccacagtgtagttcttacgacaagtcgcaacactggtattactgaaattattggtggcagcaagcaattacaacctctatcagcaactatctctaaaaatctactctgcaaaaggttatttggatcggcaggcaagtgtccttctgaactagtaaatatatgtgacaatcttgtagaagaatgtggtggaatactatctgtgatcgacgaaactgtgacattgcttgcaagtataccaccaacagtggagaactgggaggcagtgtacgccagaagaatgttggatcggtcttatcctggtttaactgacagtctaaagaattgcttactctattttactatgtttcgaagaggacatgagattagtggagaacacttaatatgtgcatggatagctgaaggttttgtacatgggcaagaggtagcagagacctaccttagtgatctagtaaaaaagaaattaatcgatgcagtggaggttgatgctggaggaaaggtcctcacgtgccgcatgtatgacttggtgcatgactttatcgtctcaaaatcaattgaagaacgatttgtttatattttaaatgactcggaaggcagagatttgtcagaagcagttcacgttcaccagcgactatacatccagggacataataacaaagaactagacctgcaaattccttggctgccccaagtgaagtcacttgtctcctgtggtactgcgccatccatcttaaagtttaagggtctacatgttatggatttaggggcctgtgaatctttgcaggctagtcatctcaagggtataaataatgtaagttctttgagatatctggtcataggaggtaagtgtatctctggcatccctaaggaaattgcgaagctggaacatttgcggacactagatttaagtgcaagtggtctaaatgaattgccagaatatgttttcatgataagaaaattggaacgcctaattgttaatagtcagatgaagatatcatatggtattgcaaagatgtctgctttacaggagctaggcgatatcaatgtcaccgacccagagttgctgaaaagtctctgtaagctaaccaaattgagggttcttagaatttccatatggtcatgggatgatagtttgaagaactattttaaacaactgtgtgacaacttgcgttcactggttcagtgcacggagaacatccagagtctctccataatgacatgctgctccctggttttcatggatgatttgggtgagaattggacccctcaatgtctccagaagctcgaggtcggttgcagcgcatttgacatattgccaagttggtttggctcactttctagtatctccacgttaacaatcgaggtctacaagctgtcacaggacataattgatacgctcggaaggctggctggtcttggttctctatccctgacatcgaaacaagtaccaaaaggatactttgtgatcggctctgacaggttcaataagctacagagcttaaagtttgtgagcaatgcaatggtagagatgtttccacgtcaacaatcaaatggcacggaacagctcaaaaggcttatgattgtgttccatgcttcacgtacacaagatgtgaacaaagatttctgctttggtttggagaacctgtcttccctagagcatgttcgtgttgaaataatttgtttcgatgccagccataacatggtgaaaaacgcagaagctgcagttcagaaagctatatctggcacaagtatcgcaaatctggaaatacgaagacttcaggaaaatagtatgacacaggacgaagcggacctctgtgatgcagtacaagagcagaataatcagaagcaccagaaaatgaagaggtactaattttccactcctacaatacaacgatgtcaaataaaattatttctcttgtattttcttataaagttcggcccttgagagcatctccaagagaggtcttaaactaggtcctatcttcaaatataggacataagagtaaaacatggctttgagatggatcgatagaatctcagcagcaacaggtaaagagttatagtaggccttcgttaaatgtcgccatggatgtgattcgcaagattctggaggttcaatgtctgctagagctgcttcatgcccacaaccccaatttcgaacgaactcaatcttaggcaacaacgatccctctgggatgtccgattttaggtagtattctttcgaatttaacgatatgctgcagaaaggcattttagatcccacgtagctaggcgcccaggcatagccgaacagacccaaactcgcgttcggacgctcagttatgcagtcatgctaggtctcatcactgatgatgtaatgcagaatttatgcaacgggggactggcaactggaaatagcacataggtctgtagaatcagatgagtagctgaaaggttaatattctgaatttcagagttattactaatatatggctattggtgtactattctacctactgtcactaattgaaatgtctcttgttatacctatgtgtgatgatgtatttgtgaaacattccatatgcccgacataaaatcattgatattgtttatgggattattgttaagatggatgtatgattattggcattgagttgtttcaactagcccttcatcgctagctacatttctcatggatgcttgtttgaatagtacttgcaatatgcttcaactagatggaatgctcggatactgatatgctactatgtattcctattttgcagttctatggagtcccaggatgggtacctcacttctttaccgaaccaggaaagtgcagatgttgttggctccgatagtattgtggaaccactagtgaatgaaatgaactcgcagacaattaagagggatcaatccacgaatttcagtgaagatgaggacttaatgttggtttctagctaccttaatgtaagcaaagattctattactggaagggataaaaaagaaggcacattttgggaaagagtatgggaatactataacaagaataggacattcgagtccgatcatagttggtcgtcattgaaacatcgctggcttgcaattcagaaggaagtgaatatctttcaaggttactatgatgccatagaaaggaaaaatcaaagtggccagacaagtgatgacaaggtgaatcatacattttgtaccattatttatacttcttaacataacatgcaacttctcacatttgtgaatcctgtgttgttttgtagcatgctgaagcagaagtagaattccgagaaaaacaagggaaggctttttctgtattccatgtgtggatgattctaaggcatgagccaaagtgggcatttagagaatcaaagatcaaagaccagcatgaagcaaacaatgctaatactgatgctcctgccaacatttatagaccacaggggaggaaagctgagaaggaaaaggctcgtgcgagaaagcatggtggatctgatgttgatggtgatccgttcatcgaagaagtaaaaaatatgagggaagcacgggaagaaacagaacgagaccgaaagacccatgatgacaagttctatgagttggaaaagagtaagcttgaattggagcgagatcgacatgacaaagagataatgcaaacagacacaagcacaatggatgaagaatcgaaacaatacttcaagttgatgaaacaagagattttggctcgccgtttcgggagtagtcagccatagttgttagctgttggaaacttagatagtattttgtttttgccagacatctgttattgataatcttgtgaacttttacatatggcacctgtcaactacttcctcctttctaaaatattattcgttttagggtgttaatagattcatacaatatttgatgtatgtattttatatatgtgtctagattcgttgtctaagggtcttctagtctggcattgcctgtacagtcataatttgagcacttcctattttgtttggtggaaagtactgtgatcactttcattgcctctgctttattgtaccatgctaaactgggtcgattacttaagtctttataaaacaactatcatgaagcatcatataaacaaaactgagctgatttatttaagtcttgttcattgctgtagactgtaactgaatcgattctgggtatttcaagaaaactgcaacttgaacgatcagaattactggaaaattcttccggcaaaagaggacaaacgaattgctagaaaatttaatgcctttttctcgaatcaacgtaacacctttgtcccttcacgggctccttaatgcatctggtacttctttttagtactgttctatactccctctgttacaattttttttttgattttttacctcaagtttgaccagttcgacctattaaaaaacttcataattatcgttaatttttacggtgatatctttagcacataatatactttaagctaaagtatgactttgatttttcatctttttgcaattttttgaataataagagctggtcaaatttaacaaaaaaaatcaaacgaattataaattaaaacgaagatagtaatatatatatatataggagaaggtaatggaagcccagagcttccattaataccgggaagtcccggccaggcagaccccacacacattttgcgggctaggtcgacgtgcgcccgatgcgcctgtttctggttcgagcagatcagagcataaaatataaactaaaacaacataaacgactacaagttttagcgtaaattgggaatctgtttcgtaacagagcgtggcggcgtgaaaatcggccatgcagagcccgcacgcgtgttttccgggctgggccggcgtgggcccgatgcgcctatttc

SEQ ID NO: 25 - DNA, 6102 bp, Zea Mays
  source 1..6102: mol_type = other DNA
atggagttcgcaactggagcgctaggcacccttcttctcaagctctccatgctgcttcatggtgagtacaacctggagaagggcgtcaggggggacatccagcgcgtcatgaacaagctcgagcgggttcattctgtcctcggccatgttgtcgaagtgcctgtgccactgaagccacgtcctgatctggtcagaatgttggcacgcagcgtcagggagctatcctacgacatggaggacttcgtcgacaccttcctggtgcgtgtccaaggccctgaacgcaccagcaaaagaagcgccaaaatattcatgaataagacatatatggtcgtgaatcgccatgagatcgcccagaccatcaaggacttcgaagagcgcgtccagcagatagatgagcgtcgtcaaaggtacgatgttgatgctatggttccccgtgtcaaaaccttggttgatcctcgcatatttgctctgaagtacaccaaggccacggaccttgtcggcatggatgaggcaagggaggaactaatcacaaggttgaccaaggaagatgacacctccactgaacaaaggcgagtctctatcgttggttttggaggacttggcaagacagcgcttgcaaaagcagtttatgacaaacttaaagctaaaggggaattccattgcgcggcctttgtgtcggtgtctcggtttcctcagctcgaaaaattcttcaaggaattgctttatgagcttgacgagactgagtacaaggaacttattgacatcagcaccccattggaacttatgaatctagtgcatgaattccttcataataagaggtaccttattgctgtcgatgacatatgggatactgacgcatgggcaatgatacaatgggcttttcctgagaataagctaggaagcagaataatcgcaactactcgcagaattgatgttgctgagtatgtaggtggttgctatatgatgaaacctcttactcgagagaaatcaaatatattattctatggacgaatatttggctctgaaggtaaatgtcctcctgaactttctgatgcgtctgagaaaatattgaataaatgcggaggcgtgccattggctattattactacatctagcttgctggctagtaagtcaagaaacataaaagaatggtacaatgttgctgattctattggttccagaatactaaaaaacagtactgaaatggagaatttgaggaacatactgctgcttagctattatgatctaccagcacgattaaagacatgtttgttatatctgagtattttccctgaagattgtgagattgggatgcatcggttaatatggaggtggatagctgaaggttttttcaatggagaactagcacatgatgggctctttaaccttggcgaatcttgtttccacgagctcataagaagaagcatggtgcagccagtaacacttgaaggcacgggtcttgtatatgcttgtcgtgttcataatatgtttcatgatttgatcctctacatgtcacacgaagaagaatttgtttctgcagtcaatgaaaagtttggtcttctagatgttcgttctcggcggttagcattccagaacataacaaaagagcagtacagacttgtggaacatccacggctggcacaattgaggtcacttaatgccattggatgtcctatatacgcgatacctccaattgaaagctataaattattgcgtgtactggatttcgaaaattgtgcaggtattgaaggccatgatcttgttcatcttgggaaattgcatcacctcaagttccttgggctaagaaacacgtttatcggtaagctgccggaaggaatagggaacctcaagtttctgcaaacattggacctcgatggaactggtgtggaagaattacctcaagccttgcataatcttacagaattgatgtgcctaattgctgactggagaacgagagtgcccaattggattggtaacctcacgtccctgcagcacttggtgatttatcctggtgggcatgacgatgaggattctgcgagcaggtttgttaatgagctgggaaagctgagacaactaagggtgctccgttttttgataaaagcacaagatgaagggcagctgagagatttgctagagtccctatcgaatctgccagagatcgaggctatacattttgattactatggagtacagttaaatagaggtgttcagttggaacctgaaggctatgccctctctagacatattcgttccatggaattgcgctggttggagttctcaaggctgcctctttggattaatcctggacaacttcctaacctctaccacttatggctgatggtatctgatgcggaagagcgggatctggaaatccttggggggtttccagtgcttcactccctccacttgttgattgtgaatactgaacgtgaagatgtcatgacttgtggctgtggtggattcaagaatttgaaatgctgcagtataactaaaccgctgaaatttgtacatggagctatgcccaggcttgaagtcctcgatttccatttcagtgtgcaactcctaacggattcaaaccaagaatttgattttgactttggcttgggaaacctacattggcttcagcaagccatcgttcaaatcacagcccttggtgaggaggtggagtctgtggggagagcacaggtggctctgcgggatgcaatacgtacccatcccaaccgtcctacccttgaaataaacttatttgggcaaacaatacctccagagttaccaaagcaagacgacgatggagcgaaaattgtggagatatcaccggccgagagtagtcgtcaagctcaagagcgggagaaaagaagcatcgatgtggcgacgaagaaagcaacacgggtgccgtctttttacacaaagtcatcaattgatgagccaatggatcagctcataaacatgctatctgtggttgatgacgaagcctacactaagaacataaagatactatctattgtaaggtctgagggactggggaagactactctggcccaaaaagcattcgaagagctccattcgcaatttgaccgtggggcgttcgttctactaggccagaatcctgacttgaggagagtctttgctgacattctccgtggtcttgacaagcaaaggtacatagatttcccagtggcaatattggatctagtggacctgatctggctagtccgtaaatcgctcataaacaaaaggttctttattgtatttgatgatatatgtgatgtaaaagcatgggaaattataaagtgcgctttgattgaaaataacaaccacagtgtagttcttacgacaagtcgcaacactggtattactgaaattattggtggcagcaagcaattacaacctctatcagcaactatctctaaaaatctactctgcaaaaggttatttggatcggcaggcaagtgtccttctgaactagtaaatatatgtgacaatcttgtagaagaatgtggtggaatactatctgtgatcgacgaaactgtgacattgcttgcaagtataccaccaacagtggagaactgggaggcagtgtacgccagaagaatgttggatcggtcttatcctggtttaactgacagtctaaagaattgcttactctattttactatgtttcgaagaggacatgagattagtggagaacacttaatatgtgcatggatagctgaaggttttgtacatgggcaagaggtagcagagacctaccttagtgatctagtaaaaaagaaattaatcgatgcagtggaggttgatgctggaggaaaggtcctcacgtgccgcatgtatgacttggtgcatgactttatcgtctcaaaatcaattgaagaacgatttgtttatattttaaatgactcggaaggcagagatttgtcagaagcagttcacgttcaccagcgactatacatccagggacataataacaaagaactagacctgcaaattccttggctgccccaagtgaagtcacttgtctcctgtggtactgcgccatccatcttaaagtttaagggtctacatgttatggatttaggggcctgtgaatctttgcaggctagtcatctcaagggtataaataatgtaagttctttgagatatctggtcataggaggtaagtgtatctctggcatccctaaggaaattgcgaagctggaacatttgcggacactagatttaagtgcaagtggtctaaatgaattgccagaatatgttttcatgataagaaaattggaacgcctaattgttaatagtcagatgaagatatcatatggtattgcaaagatgtctgctttacaggagctaggcgatatcaatgtcaccgacccagagttgctgaaaagtctctgtaagctaaccaaattgagggttcttagaatttccatatggtcatgggatgatagtttgaagaactattttaaacaactgtgtgacaacttgcgttcactggttcagtgcacggagaacatccagagtctctccataatgacatgctgctccctggttttcatggatgatttgggtgagaattggacccctcaatgtctccagaagctcgaggtcggttgcagcgcatttgacatattgccaagttggtttggctcactttctagtatctccacgttaacaatcgaggtctacaagctgtcacaggacataattgatacgctcggaaggctggctggtcttggttctctatccctgacatcgaaacaagtaccaaaaggatactttgtgatcggctctgacaggttcaataagctacagagcttaaagtttgtgagcaatgcaatggtagagatgtttccacgtcaacaatcaaatggcacggaacagctcaaaaggcttatgattgtgttccatgcttcacgtacacaagatgtgaacaaagatttctgctttggtttggagaacctgtcttccctagagcatgttcgtgttgaaataatttgtttcgatgccagccataacatggtgaaaaacgcagaagctgcagttcagaaagctatatctggcacaagtatcgcaaatctggaaatacgaagacttcaggaaaatagtatgacacaggacgaagcggacctctgtgatgcagtacaagagcagaataatcagaagcaccagaaaatgaagagaatttatgcaacgggggactggcaactggaaatagcacatagttctatggagtcccaggatgggtacctcacttctttaccgaaccaggaaagtgcagatgttgttggctccgatagtattgtggaaccactagtgaatgaaatgaactcgcagacaattaagagggatcaatccacgaatttcagtgaagatgaggacttaatgttggtttctagctaccttaatgtaagcaaagattctattactggaagggataaaaaagaaggcacattttgggaaagagtatgggaatactataacaagaataggacattcgagtccgatcatagttggtcgtcattgaaacatcgctggcttgcaattcagaaggaagtgaatatctttcaaggttactatgatgccatagaaaggaaaaatcaaagtggccagacaagtgatgacaagcatgctgaagcagaagtagaattccgagaaaaacaagggaaggctttttctgtattccatgtgtggatgattctaaggcatgagccaaagtgggcatttagagaatcaaagatcaaagaccagcatgaagcaaacaatgctaatactgatgctcctgccaacatttatagaccacaggggaggaaagctgagaaggaaaaggctcgtgcgagaaagcatggtggatctgatgttgatggtgatccgttcatcgaagaagtaaaaaatatgagggaagcacgggaagaaacagaacgagaccgaaagacccatgatgacaagttctatgagttggaaaagagtaagcttgaattggagcgagatcgacatgacaaagagataatgcaaacagacacaagcacaatggatgaagaatcgaaacaatacttcaagttgatgaaacaagagattttggctcgccgtttcgggagtagtcagccatag

SEQ ID NO: 26 - protein, 2033 aa, Zea Mays
MEFATGALGTLLLKLSMLLHGEYNLEKGVRGDIQRVMNKLERVHSVLGHVVEVPVPLKPRPDLVRMLARSVRELSYDMEDFVDTFLVRVQGPERTSKRSAKIFMNKTYMVVNRHEIAQTIKDFEERVQQIDERRQRYDVDAMVPRVKTLVDPRIFALKYTKATDLVGMDEAREELITRLTKEDDTSTEQRRVSIVGFGGLGKTALAKAVYDKLKAKGEFHCAAFVSVSRFPQLEKFFKELLYELDETEYKELIDISTPLELMNLVHEFLHNKRYLIAVDDIWDTDAWAMIQWAFPENKLGSRIIATTRRIDVAEYVGGCYMMKPLTREKSNILFYGRIFGSEGKCPPELSDASEKILNKCGGVPLAIITTSSLLASKSRNIKEWYNVADSIGSRILKNSTEMENLRNILLLSYYDLPARLKTCLLYLSIFPEDCEIGMHRLIWRWIAEGFFNGELAHDGLFNLGESCFHELIRRSMVQPVTLEGTGLVYACRVHNMFHDLILYMSHEEEFVSAVNEKFGLLDVRSRRLAFQNITKEQYRLVEHPRLAQLRSLNAIGCPIYAIPPIESYKLLRVLDFENCAGIEGHDLVHLGKLHHLKFLGLRNTFIGKLPEGIGNLKFLQTLDLDGTGVEELPQALHNLTELMCLIADWRTRVPNWIGNLTSLQHLVIYPGGHDDEDSASRFVNELGKLRQLRVLRFLIKAQDEGQLRDLLESLSNLPEIEAIHFDYYGVQLNRGVQLEPEGYALSRHIRSMELRWLEFSRLPLWINPGQLPNLYHLWLMVSDAEERDLEILGGFPVLHSLHLLIVNTEREDVMTCGCGGFKNLKCCSITKPLKFVHGAMPRLEVLDFHFSVQLLTDSNQEFDFDFGLGNLHWLQQAIVQITALGEEVESVGRAQVALRDAIRTHPNRPTLEINLFGQTIPPELPKQDDDGAKIVEISPAESSRQAQEREKRSIDVATKKATRVPSFYTKSSIDEPMDQLINMLSVVDDEAYTKNIKILSIVRSEGLGKTTLAQKAFEELHSQFDRGAFVLLGQNPDLRRVFADILRGLDKQRYIDFPVAILDLVDLIWLVRKSLINKRFFIVFDDICDVKAWEIIKCALIENNNHSVVLTTSRNTGITEIIGGSKQLQPLSATISKNLLCKRLFGSAGKCPSELVNICDNLVEECGGILSVIDETVTLLASIPPTVENWEAVYARRMLDRSYPGLTDSLKNCLLYFTMFRRGHEISGEHLICAWIAEGFVHGQEVAETYLSDLVKKKLIDAVEVDAGGKVLTCRMYDLVHDFIVSKSIEERFVYILNDSEGRDLSEAVHVHQRLYIQGHNNKELDLQIPWLPQVKSLVSCGTAPSILKFKGLHVMDLGACESLQASHLKGINNVSSLRYLVIGGKCISGIPKEIAKLEHLRTLDLSASGLNELPEYVFMIRKLERLIVNSQMKISYGIAKMSALQELGDINVTDPELLKSLCKLTKLRVLRISIWSWDDSLKNYFKQLCDNLRSLVQCTENIQSLSIMTCCSLVFMDDLGENWTPQCLQKLEVGCSAFDILPSWFGSLSSISTLTIEVYKLSQDIIDTLGRLAGLGSLSLTSKQVPKGYFVIGSDRFNKLQSLKFVSNAMVEMFPRQQSNGTEQLKRLMIVFHASRTQDVNKDFCFGLENLSSLEHVRVEIICFDASHNMVKNAEAAVQKAISGTSIANLEIRRLQENSMTQDEADLCDAVQEQNNQKHQKMKRIYATGDWQLEIAHSSMESQDGYLTSLPNQESADVVGSDSIVEPLVNEMNSQTIKRDQSTNFSEDEDLMLVSSYLNVSKDSITGRDKKEGTFWERVWEYYNKNRTFESDHSWSSLKHRWLAIQKEVNIFQGYYDAIERKNQSGQTSDDKHAEAEVEFREKQGKAFSVFHVWMILRHEPKWAFRESKIKDQHEANNANTDAPANIYRPQGRKAEKEKARARKHGGSDVDGDPFIEEVKNMREAREETERDRKTHDDKFYELEKSKLELERDRHDKEIMQTDTSTMDEESKQYFKLMKQEILARRFGSSQP

SEQ ID NO: 27 - DNA, 7129 bp, Zea Mays
  source 1..7129: mol_type = other DNA
gtttttacatcatgtttttgtttttttaaatcgggatcgaaaataatccgaacaaaactaataaaatcagttatacgataacggtcgattctagattttctcatccaacttttatccttataagcgagccttttggtccgttagatctgaattcaatggctgcccgcatgtcgcacgtaggccctatggtttatttagtgcatggtattaaaaaaacgccaagaacatgtttgaccgcatgaaaaactcttcaccaccaacagtgatgagcaggtacgtgtacaactatgtgtactcgtttggcttcaaagctgctcccgtagtttgttgaatttatactgaaatatacaagaaggtacaggtttgaaaacaaggtgccgggctagagttctggatatctcataaataatcatagtccgtgcagtgtgatgaataaatgaataaagtttggcggtgctcatttaattgctcaaatagctacgatcgagtgctgtggctgacgcccatataaaaaatcgtccataccagagatgtactcaagcatgcacccgccagcaaggaaaaaacaataaattaaacaactaccaatgcttcaggcatcatcttctcaacaactcaatatttatttaattttttctcgagtttaatcattgccgtgagtattttgtcattcatgaaaggattatatgagcttttgatcaactataagggccttttaggtaagtggctaaattatagattatctgtataacaccgactccctttgtccacaagacattgtcatggtaatattttgtcctaagtcaaataaacttaaggttgactaataaatttgtccatatttttctccgaaatagtgtactgcaaaaatatgttccaggataatctaataatgtttatttggtaatatagatatatgtatttttttacatacatatacataagtttagacaaaaacttgaatgttttgctcttttaaacatggaatatacaaataactacggtgatttgatacccggtgtcatcgctagcgattttcataatggatggtcaacctaatagtgagcgatcatgcgaataaaaggccatgttgggtggctaacaaggtggcaactaccaatctaatcttgcaaagctaatgtcaccttgtaccatctgctacatggattcccaaccctacaaagactatttggctgtcaagtgtcaaatgtactctccacgaatcattaaccagacaaggactctctaagatacatctccaagactctcaaggcgttgaaatctttgatcacatgggttttcatttctcctgtggttgaggttgagaggttaagggagagaatgagggaggggaagatgcatgtggacagtggactccatgctggattttgccaacgactcagtatgcatgtgagatggatgctaacctaagtgtactatgcagtttaataaagggcgtttgacatgtataattcaatcacattttatgatgatataaaaacatgaaggcactactagtgccccttttttcattctttttccaaagtaaaccaaggtagctcaggtctactggttaagtgagggagctgaggtttcattctggtgaccactagcggtgtcgacacggcgggatggccccacgtgggcgccagcggtgtcagtgacggagagctcctcagatctgaactccagtgggcggcgacccttcaccgcggaggttgacccccaaaccgccgtctcgcactctcccgcctccctataactaatctcctcctttccactccccgccgcgcgaaaccacacgcggcggccgcctcctccaccacgccgatggcgacccctcactcacccagacccagctgaacaactccctccaccaagcacacgcaagcaggagaaggagcaggagcaggagcaggagccgagggcggcgacagagaatagaggtagaggaaggcgatcgacgaggagctcggtctcacctgctagcggcggcgaccatggaccggatgctgctcgaccagctggccggcgaggccctgcgggaggtgctgcacgcggtgcagggcaccctgttctgccgctccaccgccgagcgcctgcgccggagcgtcgagccgctgctgccgctcgtccagggcctcggcccgcacagcacccagcgctccgcgggggagctcggcgagctcgcggcgcgggtcagggaggcgctcgacctggcgcgccgcgccgccgcgtccccgcgctggaacgtctaccgcgccgcgcagctgtcgcgccggatggaggcggccgaccgcggcatcgcgcgctggctggagcgccacgcccccgcgcacgtcatcggcggcgtgcgcaggctccgcgacgaggccgacgcgcgcatcggtcgcctcgagcgccgcgtcgaggagatcgccgccgccaccgcgcagccgccgccccccgccctctccgtccccgtcgcgccgccgccgcacaagggcgtgcccatgccgatggaggcgccgctcgctaagcccgccttcgtcgctatgacgaaggaggtgccgcagcacaagggcatggctatgtcggagccggtgccggcgaaggcggcgcccgccaaagccggggtgatggccatggacatcgccgacggacacgaagacgcggaggggatggttggcggcggcgtcaaggtggccaaggaaaaggtgaaggagatggttatgagcggcggcggcagctgggaggtggtcgggatctccggcatgggcggcagcggcaagaccacgctcgccatggaggtcttcagggatcacaaggtccgaggtaagcagaacatcgaccaccagatccaaaccttcttttccttcagttctttccaaatcctgtgcaaaggtcgcgtctttcacaggaatcgtcctgtccttccctgcaaaaattgcatcttccccatgaatagtgggtgctaaattctccaaattggcggcatagcatcgtaggtggttggtttgcgtgcgtgtcgtgcaattaggtaaaggcgaggttgatgctttcgtttttccccaaaccacatgttcggtcaaatttggcgctttgaccaccagctagtgacaactgtactgttgtgatggggtttcagattgcttttgtgaattaccatgcttggacttgagtaaccttatcgtgtcgtgttcatggaccatggtggctacttaatcttaaatcaagatacgtatctgcttaacgaaccgcacatgagactaatcaaatctatttcacatagaataaacccaattcagatccaagtaacctgcttttttgaagaaaaaaaaatctggagctgtgctaattttaggatctcctttcagtgcaaggaattgggatgacgatgttgatttagacctagtttaggtactctagtattgaccataatctatatatattgaggtggattaaggtgtaacttaaactaatttacaccccaatccactttaacacatgtggattgaggtcaataccagaatacccctaatgccgcaacccgggtttgtagctccatcatctctgttttccaccaaaaaactcatagtaaacttaaattcaattgtcacaacaacatatattcaacgattttaagtgactgctatcccaaaccgtagctgatcttagggcatgtacagtggagagacaccaaaacggttctccaagcacaggagacaactaagagactctattgtacagtggagtgtctataaacgtagtctattaataaatacaaaattaaatgtatttgtatagcatcagatcgatagaacagacgacaaattcgtacagtgggaagtgaggcgtctgttgctacttggtttacgagccagaggcgtctcttcacggagagacggctctaagatttttttgcaaataaccccttaaacaccttaagagcctccacattaaacaccactgtacatgtccttaacttcatggtatgctttgtggtcaacatggtgattatggatctcatcaaaagcccatggctacatatctgctccctgtttgcaagctctctctctcccccaccccttttcatgattctgacatagtttctttttttctacagcctacttcaacgacaggatcttcttcgagacgatctcgcagtccgcgaatctggaggccatcaagatgaagctgtgggagcagatcagcggcaacatggtgctgggtacatacaaccagatcccagaatggcagctcaagctaggaccaagggaccgaggacccgtccttgtgatcctcgacgatgtttggtctctcccgcagcttgaggagctcatcttcaagttccctgggtgcaagaccctagtcgtatcaaggttcaagttccccacgctggtgaaacagacgtatgagatgcagctgctagacgaggcggcggctctgtccgtcttctgccgcgctgcgttcgaccaggagtgtgttccgcagaccgccgacaagagattggtcaggcaggtctctgcagagtgcagaggtctccctctggctctgaaggtcatcggcgcgtcgctgcgcgaccagcctccgaagatttggctcagcgccaaaaaccggttgtctcgaggagaggccatttctgactgccatgagaccaagcttctggagaggatggcggccagtgtcgagtgcttgtccgagaaggttagggactgtttccttgacctgggctgcttcccggaggacaagaagatccccctcgacgtcttgatcaacatctggatggagatccatgaccttgatgagccagatgcttttgccatcttggttgagctttcgaacaagaaccttcttaccctcgttaacgatgcacagtacgtatcatcggacatttatgtgcttcaaaatgttcagaacttagatatcccaataacaggtttttctaactctgctgttctatacgtgcaggaacaaggctggagatctgtacagtagctaccatgactactcggtgacacagcacgacgtgttgagagatcttgctcttcacatgagcgggcgtgacccgctcaacaagcgcaggcggttggtgatgccgagaagggaagaaacacttccgagggattggcagaggaacaaggatgctccgtttgaagctcagatagtctccattcatacaggtatagcgttagtagttaattgttcttcattacatttgtagatattcatcgctaacaactcgtcatccaacttatttagtgcgcttattctgaattcctactgaaatttccaactatttccaaaactccaggcgaaatgaaagaatccgactggttccagatgagcttccccaaggcagaagtgctgatcctcaacttcgcgtcgagcctgtactacctgccgccgttcatcgcgacgatgcagaacctgaaggccctggtgctgatcaactacggcagcagcagcagcagcgcagccctggacaacctctccgccttcaccacgctgagcgggctgaggagcctgtggctggagaagatcaggctgccgccgctgcccaagacgacgatcccgctgaggaacctgcacaagatctcgctcgtgctctgcgagctgaacagcagtctaagagggtcgacgatggacctgtcgacgacgttcccgcgcctgtccaacctgacgatcgaccactgcatagacctcaaggagctgccgccgagcgtctgcgagatcgggtccctggagaccatctccatctccaactgccacgacctcaccgagctgccatacgagctggggcggctgcgctgcctcagcatcctccgcgtgtacgcctgcccggcgctgtggcggctgccggcgtcggtgtgcagcctgaagcggctcaagtacctggacatctcgcagtgcatcaacctgacggacctccccgaggagctcggccacctgacgagcctggagaagatcgacatgcgcgagtgctcgcgcctcaggagcctccccaggtcgtcgtcctcgctcaagtccctcggacacgtcgtgtgcgacgaggagacggcgctgctgtggcgtgaggccgagcaggtcatccctgacctccgcgtgcaggtggccgaggagtgctacaacctggactggctcgcggactgatgtctcgttcctggatggatgctgcggcgtccaaactgaccttgtacatatgtgatgttgcttccccccaaaccgagcgtagggaaactcatcaatccacctgccaattttgacgtgttcagtcagtgttcgtcatgcatccatccactctttattcttgggtggcaaagcctccccttcatccaaaatacaaatgcacacaaaacaaataaccacagaaaacacaactgtacaaaaggctaaagatacaacattccgatttttcagctgcagcaatgccagcaaagagaggccaagcagggttctcaaaatgcaggaactacataggtacacagtggatagctgtacagtaacgaactaaggatcaacggttagaatcagcaatggcgtcaaaacgatcaccatgcaacagcttgaacaccgatcacttcttaagcttgagctgttcgtccagagcatcagcgagtacttcttaaacatgacctgatctacagtttccaccccaaccgttcgtggttacgtttcacatactatcactccactgctcccgatgtcgaggactcgaggtccaggtccatgtcgtcgtcaaggacgtcgtcgatgcacagaaactgggtcgtccagcgggcgccatcggccggcactgttgtctccgccatgctctccgactcctcgtagttcactgtgccgtcgcccatgaggtcagctgggaggttcctgacgaaccaaggatgactctttatctcaggcatcgtcgtgattctctgcaaagagcatgcgttatggcacatcgatctgaaagttctgaacaatcaaagtgactgacgctgcaaaggggaataagctagtatggcatcattcacatctgctgggttggccaaaaaaaaaaccttcgcattctggagatacgcgggcgtagtcaggaaccgcgtaccgaacacccagtatattattctgcaaattggcatcggcatttagatgactagattttggatat

SEQ ID NO: 28 - DNA, 2616 bp, Zea Mays
  source 1..2616: mol_type = other DNA
atggaccggatgctgctcgaccagctggccggcgaggccctgcgggaggtgctgcacgcggtgcagggcaccctgttctgccgctccaccgccgagcgcctgcgccggagcgtcgagccgctgctgccgctcgtccagggcctcggcccgcacagcacccagcgctccgcgggggagctcggcgagctcgcggcgcgggtcagggaggcgctcgacctggcgcgccgcgccgccgcgtccccgcgctggaacgtctaccgcgccgcgcagctgtcgcgccggatggaggcggccgaccgcggcatcgcgcgctggctggagcgccacgcccccgcgcacgtcatcggcggcgtgcgcaggctccgcgacgaggccgacgcgcgcatcggtcgcctcgagcgccgcgtcgaggagatcgccgccgccaccgcgcagccgccgccccccgccctctccgtccccgtcgcgccgccgccgcacaagggcgtgcccatgccgatggaggcgccgctcgctaagcccgccttcgtcgctatgacgaaggaggtgccgcagcacaagggcatggctatgtcggagccggtgccggcgaaggcggcgcccgccaaagccggggtgatggccatggacatcgccgacggacacgaagacgcggaggggatggttggcggcggcgtcaaggtggccaaggaaaaggtgaaggagatggttatgagcggcggcggcagctgggaggtggtcgggatctccggcatgggcggcagcggcaagaccacgctcgccatggaggtcttcagggatcacaaggtccgagcctacttcaacgacaggatcttcttcgagacgatctcgcagtccgcgaatctggaggccatcaagatgaagctgtgggagcagatcagcggcaacatggtgctgggtacatacaaccagatcccagaatggcagctcaagctaggaccaagggaccgaggacccgtccttgtgatcctcgacgatgtttggtctctcccgcagcttgaggagctcatcttcaagttccctgggtgcaagaccctagtcgtatcaaggttcaagttccccacgctggtgaaacagacgtatgagatgcagctgctagacgaggcggcggctctgtccgtcttctgccgcgctgcgttcgaccaggagtgtgttccgcagaccgccgacaagagattggtcaggcaggtctctgcagagtgcagaggtctccctctggctctgaaggtcatcggcgcgtcgctgcgcgaccagcctccgaagatttggctcagcgccaaaaaccggttgtctcgaggagaggccatttctgactgccatgagaccaagcttctggagaggatggcggccagtgtcgagtgcttgtccgagaaggttagggactgtttccttgacctgggctgcttcccggaggacaagaagatccccctcgacgtcttgatcaacatctggatggagatccatgaccttgatgagccagatgcttttgccatcttggttgagctttcgaacaagaaccttcttaccctcgttaacgatgcacagaacaaggctggagatctgtacagtagctaccatgactactcggtgacacagcacgacgtgttgagagatcttgctcttcacatgagcgggcgtgacccgctcaacaagcgcaggcggttggtgatgccgagaagggaagaaacacttccgagggattggcagaggaacaaggatgctccgtttgaagctcagatagtctccattcatacaggcgaaatgaaagaatccgactggttccagatgagcttccccaaggcagaagtgctgatcctcaacttcgcgtcgagcctgtactacctgccgccgttcatcgcgacgatgcagaacctgaaggccctggtgctgatcaactacggcagcagcagcagcagcgcagccctggacaacctctccgccttcaccacgctgagcgggctgaggagcctgtggctggagaagatcaggctgccgccgctgcccaagacgacgatcccgctgaggaacctgcacaagatctcgctcgtgctctgcgagctgaacagcagtctaagagggtcgacgatggacctgtcgacgacgttcccgcgcctgtccaacctgacgatcgaccactgcatagacctcaaggagctgccgccgagcgtctgcgagatcgggtccctggagaccatctccatctccaactgccacgacctcaccgagctgccatacgagctggggcggctgcgctgcctcagcatcctccgcgtgtacgcctgcccggcgctgtggcggctgccggcgtcggtgtgcagcctgaagcggctcaagtacctggacatctcgcagtgcatcaacctgacggacctccccgaggagctcggccacctgacgagcctggagaagatcgacatgcgcgagtgctcgcgcctcaggagcctccccaggtcgtcgtcctcgctcaagtccctcggacacgtcgtgtgcgacgaggagacggcgctgctgtggcgtgaggccgagcaggtcatccctgacctccgcgtgcaggtggccgaggagtgctacaacctggactggctcgcggactga

SEQ ID NO: 29 - protein, 871 aa, Zea Mays
MDRMLLDQLAGEALREVLHAVQGTLFCRSTAERLRRSVEPLLPLVQGLGPHSTQRSAGELGELAARVREALDLARRAAASPRWNVYRAAQLSRRMEAADRGIARWLERHAPAHVIGGVRRLRDEADARIGRLERRVEEIAAATAQPPPPALSVPVAPPPHKGVPMPMEAPLAKPAFVAMTKEVPQHKGMAMSEPVPAKAAPAKAGVMAMDIADGHEDAEGMVGGGVKVAKEKVKEMVMSGGGSWEVVGISGMGGSGKTTLAMEVFRDHKVRAYFNDRIFFETISQSANLEAIKMKLWEQISGNMVLGTYNQIPEWQLKLGPRDRGPVLVILDDVWSLPQLEELIFKFPGCKTLVVSRFKFPTLVKQTYEMQLLDEAAALSVFCRAAFDQECVPQTADKRLVRQVSAECRGLPLALKVIGASLRDQPPKIWLSAKNRLSRGEAISDCHETKLLERMAASVECLSEKVRDCFLDLGCFPEDKKIPLDVLINIWMEIHDLDEPDAFAILVELSNKNLLTLVNDAQNKAGDLYSSYHDYSVTQHDVLRDLALHMSGRDPLNKRRRLVMPRREETLPRDWQRNKDAPFEAQIVSIHTGEMKESDWFQMSFPKAEVLILNFASSLYYLPPFIATMQNLKALVLINYGSSSSSAALDNLSAFTTLSGLRSLWLEKIRLPPLPKTTIPLRNLHKISLVLCELNSSLRGSTMDLSTTFPRLSNLTIDHCIDLKELPPSVCEIGSLETISISNCHDLTELPYELGRLRCLSILRVYACPALWRLPASVCSLKRLKYLDISQCINLTDLPEELGHLTSLEKIDMRECSRLRSLPRSSSSLKSLGHVVCDEETALLWREAEQVIPDLRVQVAEECYNLDWLAD

SEQ ID NO: 30 - DNA, 2943 bp, Zea Mays
  source 1..2943: mol_type = other DNA
atggaggctgccctgctgagcgggttcatcaaaaccatcctgccaaggctcttctcactggtacaagggagatacaagctgcacaagggcctcaagagcgacatcaaatcgctggagaaagagctccatatgatcgctgttacaatcgatgaacaaatctcgctggggaggaaggatcagggagctgtgctgagcctctcaattgatgagctgcatgaactggctcaccaaatcgaggactccatagatcgcttcttgtaccatgtgaccagggagcagcaagcatccttttttcgtcggactgtacggtcgccgaagactctgttgtcacgtcagcggctggctgccgaggttcagttcctgaagaagataccggaggaggcgcaccagcgagagaagaggtacagggtcttcgccggcctttcttcctctacccggcacactgaatcgtcttcctgttcgtctgtatctgatccgcacacacttaaggccgacgtcgtcggcatcgacggtcccagggacgagcttgtgcagcagttaaccgaagaggcagagggcctaacaaagcagctcaaggtgatctccatcgtcgggatccatggctccggcaagaccgtccttgccagagaggtatacgagagcgacgtcggccggcagttcagtctccgggcatgggtttctgctactgacagaggtccgagagaggtgctcatggagatcctccgaaattttggtaggccagtggtggatagctctagtattgaccagcttacggtagatctcaggaaacacttgggtgagaaaagctatttcattgtaatcgatggcatgcaaacagatcagtggagcaccattgaaactgccttcccagaaaacaatgttgttagcagcagagtaattgttacaacaacaatccggtcagtagctaattcttgcagctcttctaacggttatgtgcacaaaatgaaaagacttagtgacgaacactcagagcaattgtttatcaagaaagcttgcccaacaaaatattcaggttatactcgaccggaatcaaaagaagttctgaagaaatgtgatggtcaaccacttgctcttgttactatgggccaattcttgaggaaaaatggttggcccacaggacccaactgcgaaaatgtgtgtagagatcttagacgacatctggagcaggatgatacattggagagaatgcgaagggtgcttatccacagcttatctagtcttcctagccatgttcccaaagcctgccttttgtattttggtatgtttccatgtgatcatcccataaagaggaagagcctgatgaggcgatggttagcagagggatttgtacaaacacagccttcatctagtgaaaacttcaacaccctcatagaccggaatattattgagcccatcggcatatgtaacgatgatcaggtaaagacatgcaaaacatatggcatgatgcacgagttcattttgttaatgtccacctcccatgacttcattaccctgctttgtaataataaagttgaacacaaatatgtgcgtcggctttctctccatcatcatagtgctacaagtggcagtttttcggtcatcgacttatctcttgttagatctctgatggtttttggggaggctggcaaaactattttgagtttccgaaagtacgagctattgagagtcttggatcttgaacaatgtaccgacttggaagatgatcacctcaaagacatatgcaacctttttcttatgaaatatctaagcctcggagaaactattagaagtcttccaaaggagatagaaaaactgaagctcttggagacacttgacttgaggagaacaaaggtgaaaacactacctatagaggtcctcctgctcccctgtttactccatctgtttgggaagttccaattttctgataaaatcaagataacaagtgacatgcagaagtttttcttaactggacagagtaacttagagacactttcaggatttatcacagatgggtctcaaggattgccacagatgatgaattacatgaatttaagaaagcttaagatatggtttgagaggagtaagagaagcaccaacttcaccgatcttgtgaatgctgtccaaaagttcatccatgatgacaaagagagcaatgatccacgttctctatcacttcatttcgatgacggcactgaaaacatcctgaactctttgaaggctccttgttaccttaggtcattgaagttaaaagggaatttgctggaacttccccagtttgtcatatcaatgcggggtctccgggagatatgcctttcatcaacaaaattgacatcgggcctccttgcaacactcgctaacttgaaaggcttgcagcatctcaagctgattgcagatgtccttgaagattttatcattgaaggtcaggcattcctggggctgctacacctatgttttgtcctagaacgtgccaccttaccaataattgaaggaggagctttgccgtacctcatctcacttaagctaatctgcaaagatctagttggcctcggtgacatcaaaatcaaccgcctcaaatgtcttaaggaagtcagtctagatcatagagtcgcttcggaaacaagagaaatctgggaaaaagctgccgagaagcatccaaaccggccgaaagtattgttggtcaactcatctgatgaaagcgaaattaaggctgtagactgttctgttgcttcaagaccagctgtgagtgaggctaatggaacttctcccatgtcagaggttgatgtacgagaggatgacattcagatgatacttaaccaggggctctctgccgctgctgagaaacagatgaattgtgcagttcagccaagttcaaaagctgaactgaactctgatttcaataatattagtttcccagaggttgcgcttggtttaaccgagctgtga

SEQ ID NO: 31 - protein, 980 aa, Zea Mays
MEAALLSGFIKTILPRLFSLVQGRYKLHKGLKSDIKSLEKELHMIAVTIDEQISLGRKDQGAVLSLSIDELHELAHQIEDSIDRFLYHVTREQQASFFRRTVRSPKTLLSRQRLAAEVQFLKKIPEEAHQREKRYRVFAGLSSSTRHTESSSCSSVSDPHTLKADVVGIDGPRDELVQQLTEEAEGLTKQLKVISIVGIHGSGKTVLAREVYESDVGRQFSLRAWVSATDRGPREVLMEILRNFGRPVVDSSSIDQLTVDLRKHLGEKSYFIVIDGMQTDQWSTIETAFPENNVVSSRVIVTTTIRSVANSCSSSNGYVHKMKRLSDEHSEQLFIKKACPTKYSGYTRPESKEVLKKCDGQPLALVTMGQFLRKNGWPTGPNCENVCRDLRRHLEQDDTLERMRRVLIHSLSSLPSHVPKACLLYFGMFPCDHPIKRKSLMRRWLAEGFVQTQPSSSENFNTLIDRNIIEPIGICNDDQVKTCKTYGMMHEFILLMSTSHDFITLLCNNKVEHKYVRRLSLHHHSATSGSFSVIDLSLVRSLMVFGEAGKTILSFRKYELLRVLDLEQCTDLEDDHLKDICNLFLMKYLSLGETIRSLPKEIEKLKLLETLDLRRTKVKTLPIEVLLLPCLLHLFGKFQFSDKIKITSDMQKFFLTGQSNLETLSGFITDGSQGLPQMMNYMNLRKLKIWFERSKRSTNFTDLVNAVQKFIHDDKESNDPRSLSLHFDDGTENILNSLKAPCYLRSLKLKGNLLELPQFVISMRGLREICLSSTKLTSGLLATLANLKGLQHLKLIADVLEDFIIEGQAFLGLLHLCFVLERATLPIIEGGALPYLISLKLICKDLVGLGDIKINRLKCLKEVSLDHRVASETREIWEKAAEKHPNRPKVLLVNSSDESEIKAVDCSVASRPAVSEANGTSPMSEVDVREDDIQMILNQGLSAAAEKQMNCAVQPSSKAELNSDFNNISFPEVALGLTEL

SEQ ID NO: 32 - DNA, 4287 bp, Zea Mays
  source 1..4287: mol_type = other DNA
atggaggctgcagtaagttcctccacgggggccatgggccctcttctgaggaagctcgagctcctacttgctcccgaatcccggcttcggaagcgagtcaaggatggaatcggactcctcaaggaagatctagaagaagtaagctttcccctcgtcgatctatcgatgctggaaactcctagtcccagggctaagtgctggatggaggaggcgcgggaactgtcttatcatgtggaagacttcgtcgatgaattgatgctgatgctcacagacgccggagccaacataagggctgtcaacaggcatagggttggtcgcgtgaagattgctctgcttacagcgccgccgaggcgcagcggcagcacaagggtcactaagatcgctgaattgagggctctagtgtggcaggcgaccgagcgcttcgaaaggtaccagcttgaagattattgctccagcccgagcgatatgaatttgatcacccagcaccgccgggctccggcattgtatggagatgaggccaatcttgtcggaatcgaggcctccaggatcaaactgattgaaatgcttactggggaagccgaacagcaaccgaaagtggttttcattgttggacctgttggtgttggcaagaccactcttgcgaaagaaattttcggtgaacttcgaggcaaatttgagttgcgagcatttgtacatgcttcacgaaagcttgacatgagaaggcttcttgggggcattctttcccaagttcagccgcaccaccaactaccctctgttgctggtacagtgcagatcctcattgacagtattcaggaacaacttcgagataagaggttcttcattgtaattgatggtttgtgggaagaaacagcatgggacattgtaagagatgcttttccggagggcaataattacagtagaattgtagcaactacagaaaacatgaatgtagctcttaagtgttgcagttatatgacatataacattttgaagatgaaacctcttggcattaaagactctgcatatttattcttcaatcgagtttttggctctgaccaacaatgccctgatgaactgaaagaagtttcatatggtattataagaaaatgttgtggtcttccactgtccatcatccatgtagctggtcttttagcaagcatagactactcagggctatggtatcatgtacatgaccgtttatactccattcttaatagaagccatacagttgaagagattcagaaaaaaatattagaccttagttacaatagtcttcctcattgtttgaagacatgcctgctgtatttcaatatgtacccagagggttacataatgtggaaggttcatctggtgaaacaatggatagctgaaggttttatcaacccagctgaaggaaaagacagagaggagattgcagagggctattttgaggagcttgtcagtaggggaatggtccaacctatgaaaattgactacaatggtgaggtgttgtcgtgtacagtgcaccatactatatttgatctcattaattacaattccaaggaagaggaatttattgctggaatagattactctcaaccaataacaggacttgctacaaaggcccgtcgactgtccttcagattcagtagtgccaagtatgcaaagcaaccaacaagaataacaacgtcacaattgcggtcacttggattctttggattcagtaagtgtatgcctccaattgtggaatttaagcatctccgagttctagtccttgacttttggggaagtcatgatggacacatgagtttgaacctctcaaggatttacatattatttcaactgagatatctgaagatttccggtgatatcatggttgaactaccagccaagatgcaagggctacattatttggaaacactagagatagatgcaagactatctgcagttccattggatattgtccatcttccaagtttgttgcatcttagtctccgagctgcaactaagttaccagatgggattggccacatcaaatccctaagtacgctattgtattttgacctcagatgtaactctgaagacaatatacggagcttaggacagctgacgaaccttcgacatcttcatctaacctgttctacagttctctccagtgaccacctgaagagaaagctgatacctctagccttttctcttgggaaacttggcaatctcaaatctctcaccctgactcctgatgccttaagaacaaccattttgtttgatatctcgagcggtatctcctctccttccatctttcttcagaaacttgagttgttgccaccaatttgcttcttttccagactgcccgcttgctttggtgaactgcacaaactccgcattttgaaaattgtggtgaaagaactgcagggaaatgatattaacaacattgctggattaccttccctagtgattttctcactgtatgtgcggacagctctgactggaactgtgatcttcagcaccatgtcattcccagctctcaagtacttcagattcacatgtggtgtgacgtgcttggcttttcaggaaggagccatgcacagacttcaaaggctcaagctttgtttcaatgcccatggaggcaagaaccatagccgagtgattgacggcattgagtacctgttaaaccttcaggaggtttctggacaaattggggtcctcccaggtggtgatgaatccaatatgagggttgttaagttgtcatttgaagacaccattagaaagcatccaaggtgtcttagattcaacctacaattggtagattttatagaagaagaatatcctcctttagttaagttgcatcagaggcagcaggatgaatatgagattgaagagaatgactccgctgatggtaataccaaacacactgatggcaggtatggattcaacagattcatattcagtaattcttcactcgtggatctagaagcagactttcaagcacaaagtaggagtactagcaatgaccttcccatcccatataacaaacagttggattggcgcaaagcagtaaatcatatcaggtacaagcgtctgtcgcgctcctgcctcacattcctagtcagtaacaaactcttgggagtcgaacaatcatttgctctctgggaagagagaaatgaactgttaggggttcatgaacatggggacactcatcacatttcaggcatagatgaatataatgaggttgaggatggggtggtagcggtggagggggagccgaaagagttgagaataggagaggccttggaggggttaactgaaacatcttgtttgtcgatgacgaccttggaggggttcactggaacatcttccttatccatggcgagtgatgatgacacatccaatacgaccatggaggaaatgttcatatcaccagatcgacaactcaagcggaagattaagtcatggatgcgcggtgcttttttgggaagcggctctttagggatggtgtatgaggcgatcagccaagagggtgtgttttttgctgtgaaggaagtatctttgcttgaccaaggaagcaaagcacaacaatctattttggcactcgagaagaaaattgaactccttagtcagcttcaacatgaaaatatagtgcactattatggaactgagaagggagagtccaaactctatatttttgttgagcttgtgacacaaggatctctttcatccctctataaaaaatataaactacaagaatcacaagtctgttggtacacaagtcagattcttaatggattggtttacctccataagcaaaatgttgttcatggagatataagatgtgccaatatattggtccatgcaaatgaatctccaaagcttgcggattttggattggcaaaagagatgtcaaatattcttacgctgagatcatgcgaaagaaatgtttactggatggcacctgagtttattaatcctaaaaagacatttggacctgcagctgatatatggagccttggttgtgtcgtcttggaaatgctaacccgacaaataccctatcctaatgttaagtggacaaaagctttatacatgattggaaaaggggagcaacctcctatcccaaactatctgtcagaagaagcgcaagatttcatttgccagtgtgtaagagttgatccagagacccggccttccgcaacacagcttttggagcacccatttgttaacaggcaatctaatcttttgtcatccttacgagtggatgacagactagaccaaatgcctattggtgccatccgtaagaatgttaaaaaaacttaa

SEQ ID NO: 33 - protein, 1428 aa, Zea Mays
MEAAVSSSTGAMGPLLRKLELLLAPESRLRKRVKDGIGLLKEDLEEVSFPLVDLSMLETPSPRAKCWMEEARELSYHVEDFVDELMLMLTDAGANIRAVNRHRVGRVKIALLTAPPRRSGSTRVTKIAELRALVWQATERFERYQLEDYCSSPSDMNLITQHRRAPALYGDEANLVGIEASRIKLIEMLTGEAEQQPKVVFIVGPVGVGKTTLAKEIFGELRGKFELRAFVHASRKLDMRRLLGGILSQVQPHHQLPSVAGTVQILIDSIQEQLRDKRFFIVIDGLWEETAWDIVRDAFPEGNNYSRIVATTENMNVALKCCSYMTYNILKMKPLGIKDSAYLFFNRVFGSDQQCPDELKEVSYGIIRKCCGLPLSIIHVAGLLASIDYSGLWYHVHDRLYSILNRSHTVEEIQKKILDLSYNSLPHCLKTCLLYFNMYPEGYIMWKVHLVKQWIAEGFINPAEGKDREEIAEGYFEELVSRGMVQPMKIDYNGEVLSCTVHHTIFDLINYNSKEEEFIAGIDYSQPITGLATKARRLSFRFSSAKYAKQPTRITTSQLRSLGFFGFSKCMPPIVEFKHLRVLVLDFWGSHDGHMSLNLSRIYILFQLRYLKISGDIMVELPAKMQGLHYLETLEIDARLSAVPLDIVHLPSLLHLSLRAATKLPDGIGHIKSLSTLLYFDLRCNSEDNIRSLGQLTNLRHLHLTCSTVLSSDHLKRKLIPLAFSLGKLGNLKSLTLTPDALRTTILFDISSGISSPSIFLQKLELLPPICFFSRLPACFGELHKLRILKIVVKELQGNDINNIAGLPSLVIFSLYVRTALTGTVIFSTMSFPALKYFRFTCGVTCLAFQEGAMHRLQRLKLCFNAHGGKNHSRVIDGIEYLLNLQEVSGQIGVLPGGDESNMRVVKLSFEDTIRKHPRCLRFNLQLVDFIEEEYPPLVKLHQRQQDEYEIEENDSADGNTKHTDGRYGFNRFIFSNSSLVDLEADFQAQSRSTSNDLPIPYNKQLDWRKAVNHIRYKRLSRSCLTFLVSNKLLGVEQSFALWEERNELLGVHEHGDTHHISGIDEYNEVEDGVVAVEGEPKELRIGEALEGLTETSCLSMTTLEGFTGTSSLSMASDDDTSNTTMEEMFISPDRQLKRKIKSWMRGAFLGSGSLGMVYEAISQEGVFFAVKEVSLLDQGSKAQQSILALEKKIELLSQLQHENIVHYYGTEKGESKLYIFVELVTQGSLSSLYKKYKLQESQVCWYTSQILNGLVYLHKQNVVHGDIRCANILVHANESPKLADFGLAKEMSNILTLRSCERNVYWMAPEFINPKKTFGPAADIWSLGCVVLEMLTRQIPYPNVKWTKALYMIGKGEQPPIPNYLSEEAQDFICQCVRVDPETRPSATQLLEHPFVNRQSNLLSSLRVDDRLDQMPIGAIRKNVKKT

SEQ ID NO: 34 - DNA, 10808 bp, Zea Mays
  source 1..10808: mol_type = other DNA
gtcaaccccctcaatgtttcgtttcgtaggacgttgcggggtcataacttatctttgtagtatgaccttattaatcgggtggtgctcactccgctgagctctaatagagatgtctttaagtggagggaaacatcttctggtcagttcacggtgcaatcaatgtatcaggctctgattaataacggtcaaatgtttaatcacaagctgatttggaaactgaatttacccctaaagattaaaatctttttgtggtatttggttaaagggattattctaactaaggataacctcatcaaaagaaattggaatgggaacaaaaaatgtgggttttgtaatactgatgagtctattcaacatttgtttatagaatgtcatgttgctcgccatatgtggaggttgtttcacttctgttttggtatgagtgcaccgaggtctgttcgtcatatttttagtacgtggatcaccggtattgatttaaaaactaagcgtcttgttattacaggtgtctcagtgttttgctgggctatttggataagtaggaatgacttagtttttaataatgtttcaagttttacttatctgcaggttctttttagaggtacacactggcttagattctgggctcagctacaaaaggatgaagctgatggagttttaataaagaatgtctgtcgtcgcctggaatcggtggccatgcaattttgtgttaattttggttggaggttttctaataggattgccttgtaatcatctcactggttttattaagtttaaagaacctttagtgtgttagtgtgtggctgtgtggaggggtatgtcccttcattgagcagtgtgatataacaatttgtgttggccctaacctcttttgaggggaaagccggaactttctctccattatcaaaaaaaatataatgactaaaatactcatttctcctttgacgtcagttttctaggcaagattggagtaaatgttatcctttaattcttttagcacccatgtgagggactagatactaaatccaattagtatctactttagtccatctgtttgacaaaatagggactaaactagactaaaaaccagaaactaaatattagtcagtctaacgaaacggggcctaattagctatcgttctacgcgaggatctacgttgtttgatagatggcgcgctgctaaaaacatgacccgtcgggcgataacttcatccttttcaaaagtcctgtcgagcacggcactgtgcagcctatagatgccgagataagatcgcatacaagttgacttgacccgctgcctggaaaaagataagcttcgaacgaatcatgcccacacaacacattccgcaagattgattgataatcacaaagaaccaaacggggatttggaaatgaaaatccagctctgctagaccattcaatgatctgaattttgtcaccgcaggacacaggtggaacatcggccgggaatatctctgtccggtttcgctgagagatactctaggattccgaggtttgacacgtcgcaacaaccttgtccagatcaaacaacgacggtggtgcgctaatagacgaacaggatggaagcagaaaagtgacggcaagtaaatgacgagagtttgacggactcacggtcttctcttcaccggcggtcaagctaagcctagtgctgttctgtggatgtaacggtgctgtaaaagggtttataaaccccataggaggtcagccagctgagtatccagtttcaagttcagagttcaatctcacagcaccaagcaaaaaggaagcagatcaagggcagccgagcgagcagaggtaccccggtctctctttctccttccgtacataattatccaattcagtttgttacttgttaagagaattcgatagtaagtttgtagtaagcttggtcactccttgaactcagcgtagtactttctccgtaatacgccgccaagagatcattctttattttttgcaggttgtcaattacgtgccttaatatgagtcattggaagcagccgtcgaaactcgccatgcttgtagtactgctgctgctgctatgtcatgcagtggacagagtccactgctcgacgcatcataacaacagccaagattttcattctctgctagagttcaagaagggcatcaccagtgatccgcacggagtcttgagcaattggaaccccagcatccacttctgccattggcatggtgtgaactgcagttccacgcgaccatatcgagtcacggagctcaacctcaccggccaaaacttggctggccaaatcagctcctctcttggaaacctgaccttccttcatatccttcgtcttgccaacaatagtttccatggtcccatacctcttctcaacaaactccaaaacctgagcaaactcgttctgggaaacaaccttttggaggatgtttttcctgattggattacaaactgttccaacttagtcagcctacatctctatggaaaccaactcaccggtcatattccttcaaacatagactttctaacaaaactagcatatatcatccttcacagtaataatctcactgggttcatccctccaaccttgggaaacatccacacactaatggtacttgatatttcaaataatcaactaaacggaagcattcctaatgaagtttggcaaataaagaacatagaaatgttaaatctagcaggaaataacctatcaggtggaatcccagatactctccctcacttagcttttcttatgatattatcattggacaccaatatgttgggtagcacattgccatcaaacattggtgatgtgctccctaatctgaaagaactatacttaggaggcaacttttttttgggtacaattccatcttccctaggcaatgcttcaaatctagaagtcatagacctaccaaacaaccttttcagtggcacaatcccaagttcttttggaaacctttcaaagttgcagactctaaaccttgaggtaaacatgcttgtagcaagggatagtgagggctggcaattctttgatgccctggcaaaatgtagatatctcgttatactttcagtgtctcataatcatctacacggacctataccaaattcgattgctaatctgtccactagtcttcaacaactattcatgggttggaacaacctttcaggaatagttcccccaactattggaaaacttagtggcttaactgaattatcactacaaaacaacaatcttacaggtaccattgaggaatgggtcggaaagatgacaaatctaacaattttaacactacaatcaaacaacttcacagggaaaattccaccttcaattggcaatcttacacagttgacaaatttctctgtagctgaaaacaatttcttcgggtctgtaccatctaacttgtggaatctgaaatcaatgttgtatttagaccttagtcataacaatttccaagggagcatacttgtttagtttggtaacttaggactcgtctcgctaaaaatttcatcgaacaaattcattggagaaaatcctgaaactttaggacaacttgaaaatatacaaaccattcaaatggaccaaaacattctcggaaacattccgtacacattcaaaagtctatatagcttgagcttgctcaatctatcccataacaatttatccggcctcatgccaacttttctaagtggtctaaatctcactaaactggacctatcctacaataatttccaagggaaaataccaagcattggtctattcgataatcctgcaattgtttcactagatggcaatccaggattgtgtggaggagacatggatttgcacatgcctccatgccatgtcgctgcttcgagaagagtaggcagaacaagcttattgatcaaaatattgatcccaatatttgggttcatgtcactcgtattgctgtgtactttttattcctagagaagaggacgtcaagaagagcatatagatcagagctatcttatggtgagcattttgagaaagatacttataacgatttagctcaagcaacacgggacttctcagaatccaacctgattgggagaggaagctatggttcggtgtatcgagggaagctgaaggaaagcaaaatggaagtggcagtaaaggtttttgaccttgagatgagaggagcagaaagaagcttcgtgttagagtgtgaagcattgtgaagcattcaacaccggaatcttcttcccatcataactgcttgctcaacagtagataatataggaaatgttttcaaagctttaatttatgagttcatgcctaatgggagcctggacacatggctacatcacaaaaaagatgtggaggctgcaaaaaatcttggcttgactcagaataagcatagtggttgatatagctgatgcattggattatctgcaccatgattgtggatggccaactgttcactgcgacttgaaacccaacattatccttctagatgatgatatgaatgctcttctgggagattttggaattgcacgcctctatcttgattatcagtcaaaatgggcaggttcaattagttcaattggtgtaaagggaacaattggatatattcctccaggtacctatagttattttagctccttttttgatcatttatgtcaatattttttataggtagaaaataagttataacacaattaaattgttaatttgtgtgagcattttccaatttagagtatggagaaggtggccatgcatcaatgtctggggatgtttatagttttgggattgtgttgctagagattttgacgagcaaaaggccaacatatcctatgtttaaggatggcctggacatcattagctttgtggagaataactttcccgatcaagtatttgaagtcattgatgctcacctcctcgaggaatgcaggaacccaactcaaggaaataccttggtacaagaaaatgagatctatcaattcttggttgatctgctgcaagtagcactttcatgccttcattcgttaccatctgaacgatcaaacatgaagcaagtagctagcagaatgcatgcaatcaaaacataacataatgaaatcacaagtaataagaacaagggtgttctatgaattgtacaataacatattgttggtacctaatttgaactaggtctaggatacctttttggccaaagggctccacaaaggaagtgttcggccgcatggaaaatagacatgtaaaacagaaataagcatttgtactgtaaataagtaatagatcagatggaaatataagttaggaagcgcttattgtgtaaggcccaaaatctgtataagaaaaaaaaaagtaaatatactactatgtataattttgaggatttcttgatcctcttttctttgagcaaattaaactaaaggaattaaagtagtcgaatgacttccaataacaattttattcctttatagaatatacctctataaactaaacacataatgataactaagaatttggtattcaagatttatatccaaaacttaaaactaaaaggttggagcaacaaataaaagagagactttcatactacttatttcctttaaaaactatatagccccttaaaatatagttgaaataaaataattcatgaggatattatgtactccaaagtagatatgtaatgaagattaccttttttaagtgtgcattcaaaataatgtttcctaaataaataaataaataccaagtgaatatctcattattgaagtatttgtcaatatatatatatataaagaaagaaggaatgcatcatgctagaattttgaatgtgcatttaaagcatagattcgaattcaaatgtgaaaagaaaaaaactggaatttgaaataaaaaaaacagcaaatataaaagaaaaacaactttcccctacacctgggccacattccctcggccctagaatatgacaagattttcgcttgagcttgtgaggagattagttgcaacataaacgaccctagaatatcaattcagggtttgttgtcccatattacatatggaacagttgtctgacggttgatctccattgcctcagaagatgattgggtgagacatgtaagaattgtgaagacgatgcttccaccatgtttggatgtggttgttcaaaagttatctattactcatcgtgatgctctagtcgggttgtctccacaaatgccaaatgcatctcgtattgaagctcctttggtagagcttccggaagaagtggttgttgtgcccgatgctcaatcggggccgcacgagtatgggatttctcgtcctctttctggcgttttgtggggcttctaatccggtggtacccccaagagatcccattgacccaggatccagttcaggatcatcctagtaagtgtctttgacacgttgttcatatccatcgttatttcgtttgattagactttttaatgtggtatttcttgattggacccgatgcaagatctccacaaatcgataatggtgcttatcttgatgtgcatgtatcatataatatggacaacatatgcagggcatccaatagtgttgatgttcagattgaggatgaggaggagccatatgaggctgcacgggccttagattctgatgatgatcgtccggttcaagaaatgaccgagcaagaaattgaactcataagacgtttgtgtcccgagcgtgaccctgcagtacatgaatttagcagtctaagtcattccacccgtgcatatgctgaaggacgtgatgatgaactgctagaggctccggataacaccgatagcattgagattaaggtaggcttacttttcaaggacctgcctacactgagacgatggctacaggaatattctgtgaatcgcaagaggccgttcaaggtgagacactcgtatgcacaacgtcgttacactgttgtgtgcgaggtgtcgaaatgtaattggagggtatgtgcctgaaggcagaaggaaaccggaaagtttaaaatcaccaaaattgtaggtccacacacttgtgcccagatagagctgagctttaagcatcgtcagttgacatctaccctaattgcgaaaaggatattggggatattgaagggtcagccaaacttgaaggtgaagtcaattatgaccatgacttcggagctgtttggttacaggatcaaatatgggaaagcatggagggcaaagcagcgagcatgcactacaggaaacgcataaattttcgtaggccggcctattttcgtcggtcggcccacgaaaatacaatgttatttttgtcggccttcgtgaccgacgaaaatgtggaatattttcgtgggccaggcaatattttcgtcggcaggccgacgaaaatacgaaagttattttcgtcggcctccacaccgacgaacattttgtgtattttcgtcggcctcagcccggccgacaaaaataaacgtttaacgggccactattttcgtcggcctcactgaggccgacgaaaatagaggccgttaatcttccatcccggcctcaccgcaccttctctctctcgctcgtcttaccttctctcacccgccctcaaccgcgccgcaccgcacgccctcgccgcgcgcccgccgagccgccgcgcagccgccgcctccccacgccgcgcccactccgagcagccgcgccctcgtcgcgccgcgccacgccgagccgcctccccacgccgcgcccactccgagcagccgtcccacgccgcggccccctccaccgttgccacggtaatggttttacttagtttatagtaattagttagctaatttacttgatataatgtatagattgatagctattcacttgttagtataaatagttagctaggtatttatatgtaggatacttgtagcattttgtttaacacattgatatcacatggttagcatattgttcattatgtagctatcgtgcagtattctgtgttggcaccatcgtgttgttgtttctttgcaggttttagaaacatcactttatagtggaggtgctgccgattttttttattgacaatagtaatttttgtacataggtgttcttccgacttgaccttctccaccgttagctggactcgtacatattctcaggtatacattgtttttgtaatttcggttaatgcggtcatttattatttactatatagttattagtaaataagtagttactatttattctcaggtatacattgttttcatactagttatttgttttctccaccgtggcgtgccgtgcacgtgccgtgcgcgcggccgtgcgcgtgccgtcttttgaatgttatttatcttttgtttaattagcctttgaaatggacggtgatcggagggtgatgtcttttgtttaatctccgagatattttttaatgtttagagttcagttttaatcgttaaccgtagcgaaccgtatgcgtcacccattcgggaacggcgaacgtcacattggcttgtgaggttcgccgttccggaattgtccacgtattttggacagcctgagagtgtaagccgatgcttgtgatttgtgacatgtgccttacgattgacgcggaattttggttgtgtaacccagttgttctccaacacaccatgttcaggcgtgtgcttggagagcagcggggttatgctaccgaaatttcacggcattcgtaggctacacgtcataaatcacaagcatcagcctacactcttgggtgggtttagggcctttacctaatagggagttcacctaagccacggacgatcgttgatgttctttgacttttgtttagcctttgaaatggacggtgatcggagggtgatgtatgacgggtggagaaaggatggggcacattcgaatgaatggatgggtgtaacaaaggcttttcttgagcacgctttcaaagatgcaactggtcgtctagtgaagtgtccttgcaatcgctgtgagaacaagtggcctcagaagaaggaagaaatggaggttgaaatgaacgctgaggagccggggcagcttccacgcgatgctcaggaattcttccggctactcgccgcgggagaagagagactgcacgagcacactcagatgtcagttcttgggactctcacaagactaatggtgataaagtcgaagcacaacatttcaaacagtgcttacaatgacatcgtccaactgatgggcgaggttctcccggagaatcataagttgccaaagaatatgtacttcacaaagaagatgttggctggtcttgggatgacatatgagaagatcgacgtgtgtcccaacagttgcatgctattctttgaggaggatgacaagctggaccgttgtaagcattgtgaagcttctagatatgtcgaggtgacaaatgatgagggtgaattggtagttacgaaggtcgtagctaagcaacttcgtcggttgcccatcattcctcggctttcaaggttgttcctcaacaaggaaatagctctgcatatgacgtggccaaagaatggtgtacgtctcatcacaaggaaatagctctgcatacaacgagccgaagaagcatcatcggatgcggatgacttttgattagtatgtttcttttagttaatcaattaagccatgtattccaattttcacattattaattttcatattatttgtttcatatactgtggtttgacattaatttatctacagttgaacatgttcaagcataggagatcgaggaggagtgggggcagcgcggccagcgaggacagctcgggcagtgggctttttcagggcacctcacagagccgacagaggcagcaacaacttctcgactgtctagacgaagtgtagggtgaggagggtgagtaggaggctcctcagcaggatgctcatgtgcagggtgaaagtcgcctagagggggggtgaatagggcaaaactgaaattcttaaaaataatcacaactacaagctgggttagcgttagaaatataattgtgtccgcgagagagggtgcaaaacaaatcgcaagcgaataaggagagagacacgtggatttgttttaccgaggttcggttctcgcaaacctactccccgttgaggtggtcacaaataccgggtctctttcaaccctttccctctctcaaacggtccctcggaccgagtgagcttttcttctcaatcacttggaacacaaagttcccacaaggaccaccacaagattggtgtctcttgcctcaattacaagtgagtttgatcgcaatgaaagaatcaaagaaagaagaaagcaatccaagcgcaagagctcgaaggaacacaagcaaatctctctcactaatcactagggcgttgtgtgtagtttggagaggatttgatcacttgggtgtgtctagaattgaatgctaaagctcttgtaagtaattgaagtgggaaaacttggatgacttgaatgtggggtggttgggggtatttatagccccaaccaccaaactagccgtttggtggggctgtctgtcgcatggtgcaccggacagtccggtgcacaccggacagtgtccggtgcgccagccacgtcaccaggccgttgggttccgatcgttggagctctgactgctgggcccgcctggatgtccggtggcacaccggacatgcactgtagagtgtccggtgcgccacttcgcgtgtgcctgacttctgcgcgctctggcgcgcatttaatgcttctgcaggtgaccgttggcgcgaagtagtcgttgctccgctggctcaccggacagtctggtgtacaccggacatgtccggtgaattatagcggagcaaattcccgaagttggcgagttcctgagacgctctt

SEQ ID NO: 35 - DNA, 3903 bp, Zea Mays
  source 1..3903: mol_type = other DNA
atgagtcattggaagcagccgtcgaaactcgccatgcttgtagtactgctgctgctgctatgtcatgcagtggacagagtccactgctcgacgcatcataacaacagccaagattttcattctctgctagagttcaagaagggcatcaccagtgatccgcacggagtcttgagcaattggaaccccagcatccacttctgccattggcatggtgtgaactgcagttccacgcgaccatatcgagtcacggagctcaacctcaccggccaaaacttggctggccaaatcagctcctctcttggaaacctgaccttccttcatatccttcgtcttgccaacaatagtttccatggtcccatacctcttctcaacaaactccaaaacctgagcaaactcgttctgggaaacaaccttttggaggatgtttttcctgattggattacaaactgttccaacttagtcagcctacatctctatggaaaccaactcaccggtcatattccttcaaacatagactttctaacaaaactagcatatatcatccttcacagtaataatctcactgggttcatccctccaaccttgggaaacatccacacactaatggtacttgatatttcaaataatcaactaaacggaagcattcctaatgaagtttggcaaataaagaacatagaaatgttaaatctagcaggaaataacctatcaggtggaatcccagatactctccctcacttagcttttcttatgatattatcattggacaccaatatgttgggtagcacattgccatcaaacattggtgatgtgctccctaatctgaaagaactatacttaggaggcaacttttttttgggtacaattccatcttccctaggcaatgcttcaaatctagaagtcatagacctaccaaacaaccttttcagtggcacaatcccaagttcttttggaaacctttcaaagttgcagactctaaaccttgaggtaaacatgcttgtagcaagggatagtgagggctggcaattctttgatgccctggcaaaatgtagatatctcgttatactttcagtgtctcataatcatctacacggacctataccaaattcgattgctaatctgtccactagtcttcaacaactattcatgggttggaacaacctttcaggaatagttcccccaactattggaaaacttagtggcttaactgaattatcactacaaaacaacaatcttacaggtaccattgaggaatgggtcggaaagatgacaaatctaacaattttaacactacaatcaaacaacttcacagggaaaattccaccttcaattggcaatcttacacagttgacaaatttctctgtagctgaaaacaatttcttcgggtctgtaccatctaacttgtggaatctgaaatcaatgttgtatttagaccttagtcataacaatttccaagggagcatacttgtttagtttggtaacttaggactcgtctcgctaaaaatttcatcgaacaaattcattggagaaaatcctgaaactttaggacaacttgaaaatatacaaaccattcaaatggaccaaaacattctcggaaacattccgtacacattcaaaagtctatatagcttgagcttgctcaatctatcccataacaatttatccggcctcatgccaacttttctaagtggtctaaatctcactaaactggacctatcctacaataatttccaagggaaaataccaagcattggtctattcgataatcctgcaattgtttcactagatggcaatccaggattgtgtggaggagacatggatttgcacatgcctccatgccatgtcgctgcttcgagaagagtaggcagaacaagcttattgatcaaaatattgatcccaatatttgggttcatgtcactcgtattgctgtgtactttttattcctagagaagaggacgtcaagaagagcatatagatcagagctatcttatggtgagcattttgagaaagatacttataacgatttagctcaagcaacacgggacttctcagaatccaacctgattgggagaggaagctatggttcggtgtatcgagggaagctgaaggaaagcaaaatggaagtggcagtaaaggtttttgaccttgagatgagaggagcagaaagaagcttcgtgttagagtgtgaagcattgtgaagcattcaacaccggaatcttcttcccatcataactgcttgctcaacagtagataatataggaaatgttttcaaagctttaatttatgagttcatgcctaatgggagcctggacacatggctacatcacaaaaaagatgtggaggctgcaaaaaatcttggcttgactcagaataagcatagtggttgatatagctgatgcattggattatctgcaccatgattgtggatggccaactgttcactgcgacttgaaacccaacattatccttctagatgatgatatgaatgctcttctgggagattttggaattgcacgcctctatcttgattatcagtcaaaatgggcaggttcaattagttcaattggtgtaaagggaacaattggatatattcctccaggtacctatagttattttagctccttttttgatcatttatgtcaatattttttataggtagaaaataagttataacacaattaaattgttaatttgtgtgagcattttccaatttagagtatggagaaggtggccatgcatcaatgtctggggatgtttatagttttgggattgtgttgctagagattttgacgagcaaaaggccaacatatcctatgtttaaggatggcctggacatcattagctttgtggagaataactttcccgatcaagtatttgaagtcattgatgctcacctcctcgaggaatgcaggaacccaactcaaggaaataccttggtacaagaaaatgagatctatcaattcttggttgatctgctgcaagtagcactttcatgccttcattcgttaccatctgaacgatcaaacatgaagcaagtagctagcagaatgcatgcaatcaaaacataacataatgaaatcacaagtaataagaacaagggtgttctatgaattgtacaataacatattgttggtacctaatttgaactaggtctaggatacctttttggccaaagggctccacaaaggaagtgttcggccgcatggaaaatagacatgtaaaacagaaataagcatttgtactgtaaataagtaatagatcagatggaaatataagttaggaagcgcttattgtgtaaggcccaaaatctgtataagaaaaaaaaaagtaaatatactactatgtataattttgaggatttcttgatcctcttttctttgagcaaattaaactaaaggaattaaagtagtcgaatgacttccaataacaattttattcctttatagaatatacctctataaactaaacacataatgataactaagaatttggtattcaagatttatatccaaaacttaaaactaaaaggttggagcaacaaataaaagagagactttcatactacttatttcctttaaaaactatatagccccttaaaatatagttgaaataaaataattcatgaggatattatgtactccaaagtagatatgtaatgaagattaccttttttaagtgtgcattcaaaataatgtttcctaaataaataaataaataccaagtgaatatctcattattgaagtatttgtcaatatatatatatataaagaaagaaggaatgcatcatgctagaattttgaatgtgcatttaaagcatagattcgaattcaaatgtgaaaagaaaaaaactggaatttgaaataaaaaaaacagcaaatataaaagaaaaacaactttcccctacac

SEQ ID NO: 36 - protein, 1254 aa, Zea Mays
MSHWKQPSKLAMLVVLLLLLCHAVDRVHCSTHHNNSQDFHSLLEFKKGITSDPHGVLSNWNPSIHFCHWHGVNCSSTRPYRVTELNLTGQNLAGQISSSLGNLTFLHILRLANNSFHGPIPLLNKLQNLSKLVLGNNLLEDVFPDWITNCSNLVSLHLYGNQLTGHIPSNIDFLTKLAYIILHSNNLTGFIPPTLGNIHTLMVLDISNNQLNGSIPNEVWQIKNIEMLNLAGNNLSGGIPDTLPHLAFLMILSLDTNMLGSTLPSNIGDVLPNLKELYLGGNFFLGTIPSSLGNASNLEVIDLPNNLFSGTIPSSFGNLSKLQTLNLEVNMLVARDSEGWQFFDALAKCRYLVILSVSHNHLHGPIPNSIANLSTSLQQLFMGWNNLSGIVPPTIGKLSGLTELSLQNNNLTGTIEEWVGKMTNLTILTLQSNNFTGKIPPSIGNLTQLTNFSVAENNFFGSVPSNLWNLKSMLYLDLSHNNFQGSILVFGNLGLVSLKISSNKFIGENPETLGQLENIQTIQMDQNILGNIPYTFKSLYSLSLLNLSHNNLSGLMPTFLSGLNLTKLDLSYNNFQGKIPSIGLFDNPAIVSLDGNPGLCGGDMDLHMPPCHVAASRRVGRTSLLIKILIPIFGFMSLVLLCTFYSRRGRQEEHIDQSYLMVSILRKILITILKQHGTSQNPTLGEEAMVRCIEGSRKAKWKWQRFLTLREEQKEASCSVKHCEAFNTGIFFPSLLAQQIIEMFSKLFMSSCLMGAWTHGYITKKMWRLQKILALRISIVVDIADALDYLHHDCGWPTVHCDLKPNIILLDDDMNALLGDFGIARLYLDYQSKWAGSISSIGVKGTIGYIPPGTYSYFSSFFDHLCQYFLVENKLHNIVNLCEHFPISMEKVAMHQCLGMFIVLGLCCRFRAKGQHILCLRMAWTSLALWRITFPIKYLKSLMLTSSRNAGTQLKEIPWYKKMRSINSWLICCKHFHAFIRYHLNDQTSKLAECMQSKHNIMKSQVIRTRVFYELYNNILLVPNLNVDTFLAKGLHKGSVRPHGKTCKTEISICTVNKIRWKYKLGSAYCVRPKICIRKKKVNILLCIILRISSSFLANTKGIKVVELPITILFLYRIYLYKLNTLRIWYSRFISKTNKVGATNKRETFILLISFKNYIAPNIVEIKFMRILCTPKICNEDYLFVCIQNNVSINKIPSEYLIIEVFVNIYIYKERRNASCNFECAFKAIRIQMKEKNWNLKKKQQIKKNNFPLH

SEQ ID NO: 37 - DNA, 7418 bp, Zea Mays
  source 1..7418: mol_type = other DNA
cggtgcagatctgcgctagggcaggaacggttgaagaagcgagctcgatgtcagtggtgagcttcccgttgctatcagtctccctctaggtcggtctagagttgaggtggcgaattgagacgtaacctcgtgtcgagccatcaactctcaccactctatttatagactacgtaacaggaacccattaaactcgtcttgggctgaacaccctcgattagggtgcgaggtcatagttagccgttgggccaactgatggagatcaattctaacactacagagtgtacatacgggtgaacccatgaaccttgcagtagagaaactgtaaggtaattttgtgtggacttaaaacaataatcattacactaatttgtgctgtgattccttaattgtctggtgcctggtgggctaggctggaattcctaaccttcactgccccgcctacacgttgcgcactttgactacacttggaaaacagtctgctggtcctagctgcgataacattgaacaggcatgagatgcaaaagcttgacctgatgccggtccgttccaaccatcgattgttaaaagatcctgatggcgatacagcaaacttgcttgacctggtgcaagtcttcgggccctacactcggttgagcagagaacgacacaatcctatgaattaaaattcaccaccagagagtactctttattaatttagctgaatagcttcctgtgactcgactcgatccatcgatattgcagagaagaccaggcctcgtctgtctagctgatgtgatgaacatgcatcaaccttgctttctacagtatctcctcctcatcctcgtgcctctgctgctgtccgcctctgcttatgatagatcaggtgagttcaccttcgatggtttctctggcaacgatctaataaccatggatggtgcagcttctgtcaccaatggcctcttgagcctcactagcgggcagaaggaattgagaggtcacgctttctatgctttccctctcgatttcacaagtgccgccgtcccaaatagctccgccgttctgtccttctctacaacctttgtctttgccatcatcggcccatatgatgatttgagtggccatggtcttgcctttgtgctttcttccaccaatgatttgtttactgccttgcctagccagtacctaggccttctcaattcatggaatgatggtaactcatcaaatcatcttcttgcaatcgagcttgataccatacagagcacccagttcaatgacatcaacgacaaccacattgggatcgacatcaatagcctgtcctccgttgcctcacacactgcaggctactacacctccaatggagagtttcatccgctgaaacttataagtagaaaaccgatgcaggtgtgggtggactatgacagcaaccatatcatgctgaatgtcacaatagcaccttacttcatgtcgtccaccaaacccagcaggccactgctctcgactatattcaatctttcctctattttgccaaccgcaacagtatacgctgggttttcctccgcgactggcacacttaactgcaagcattacgttcttggctggagcttcaagctcaacggagaagctgcatcactcaactattccgccttatctgtcaagactatccaagaaatcgcacaacaagttccaactagcgatcacccaggcagctacaaggttctgatatgcaaaattctcttaccgatcgttgcaattttcgtacttgtttctgctgttcttgttaaggtccacctgaagaggcgatcacaagaaagggctgaactagactggcagagggagtatgggcctccgtctttcacctacaaagaattgttggccgccactcatggattcaaggacaagatgcttcttggtagaggaggctttgggagtgtgtacaaaggagtgatgcctatctctaagcagatagctgctattaagcgagtgtcgccggagtcaaggcaagggatgagggaattcatggccgagatcaccatcctcggtcatcttcgccaccgcaatctggtgcagctgattgggtattgcagccataagcaacatcttcttttggtctacgactacatgccaaatggaagccttgactgctatttgcatactcaagatcatggcagcaccaatctatgctgggctcaaagattccacatcatcaaaggtatagcatctggtctcttctacctccacgaggactgggagcaggttgtcatccaccgggacatcaagaccagcaatgtccttctcgatagcgagatgaatgcgaggctaggtgattttggccttgcaagatcccatgatcatggtgctgatgctcacaccacgcatgtggcaggtacgtacggttacattgctcccgagttggccaggcttggcaaggcaaccaaagcaactgacatctttgccttcggcgtgttgatgatggaggtcacctgtgcaatacggccaatatgggtgaacaccaccgatggggagcctctggccctagcagactgggtgcttgctgcatggcaaggtggttccatcaccgatgccgtcgacccaagactggatagttatgtggatgaggagatagagttagtcctgaaacttggcttgctctgttcgcatccctcgccaaacgcgaggccttgcatgcgccttgtcatgcagtacctgcaaagcgacacaccactcccatcagactttcaaccgaatagtttactgagcattggtgtgattcaagatgaaatgcatgatcagcgagccatgtcatgtccatcagctgctataaccgatctttccaaagggagataacatcgctgtgttactttggcaataatcatctgcaatgtgctcttgcttaggactagtttggaagcaagaataatggaggagattgagagactacaatcccatactattcaattttaaacagtgatagctagtttggaaactacatttttctaagggatttctattttcctcagggaaaataaacttatttcccttaagaaaatataaatcccttaaaaaatgatgtttccaaactagccttgagggattctagtctcatcgatcccctctattccagcccttagtgttggtactgctactgcttatttgacgtgctattctatggcagagtttcgatttacttggagtaatgtgtcaaatcctatgacgttgtattactatgaacatctttcgtctgagattatccagctactattcataacgtcaatatactagatgcagtacccaaatgtactaaatgttgtacaactgtaggtctggcctctcggaatgctgtagaattttggtttaatttcatgcttcctgattattaccatggacatctaagagcttccacgagtgagaagtctagttgtatatgtttgatttagcaaattactttttgtctttttttaatgcaataaagaatttaaggtgttctcatttttaatacagtaaggaacaaaactcgaaaaaaggggaaaaacatcaatatatactagtacattattcagatcataccttgtactagtcagaactgccactctctgctgaaagggtccaagcactaaaatagcgtttgcattaatatttcttgtttaccttacactgattaatgggcatgtttatataccaattcacatcaatgactataatacagttgcttaaatttgattcatcaagaaccttgctaaattactacagtactagagtccctttgcagcaagtttttcgatttggctctttgataccacttaaattcctcttccctaagcaactcagccaatctattttgcaagcaatgttttaaatcaagctcttgagctgataaaagcactacttctgtttttttatcaagtccatctagcttattcaagattgttttcttctctttattgcaggcaccactggtatttttagcccaacctctcaaaaatttaaatttttcttctctttattgcaggcaccaagtcttcttatctttgcatgccacctctccagaggtgtatggcccccaattcttgtgatcttacattcttaaccatgtcatagaatccatctcacagtaatcaccctaattcaaatttaaattgttttttaatatgattcgagctggattcaccaaagtttaaaagaaggggggtatgatcagagatatctctattaagggctaccaccgatgataagggatacttctaatccaattctgtggtcagtaacactctatcaagtttctcatatgtaggtgtagtaaggttattagcccaagtaaactgccgcccagacatctccaactccttcagacaaagaccatcaattacgacattaaacaggaaaggccaacgatggtcaaaattgtccttattttttcgctgggttttctgagaatattgaaatcccccctattaaaagaggttgggattcatgactccctaagcagactaactccgacaagaattgttcttttttatccacttgagcaggaccatagacctctactaaagcccacttgaagcaatcacttttgttgcacaaggtgaacttaacaaaaaagtcaccctcctctatggcccctatatcaaacacgtctaagtcaattcccaataaaatacctcctgagcgaccttctagctctttgcaatgccaaagaaaattcttccctccacatagattacgcaaaaatggatcggtagacgacttacgcaccgtctccgaaattgtaatgaaagacaagttttgctctttagccaaatctgaaacaaacttatgtttcatggggtctttgaacccatcactattccaaaaaatacctttcatttgaaaccaaagtttttttcttctaccttttcgatttgtgttataagatactgcagtacgagccatagaattatgctcactgttagtatccataatttcctctagaacttcactacacatgttactaagcaataatttattcaactcatcactttcataaaactcaatctcttccttaactattgccctatctgctacttcttgttaattttgggacagagaactagagccacccgactgaaccatggactcaataaagttttgtacaacacaagaatcatttgtaaatttaaagcctaaaggatgtagattagccacaacatcatcacacgaaaaatgtaaagatgaacaagagttactcataccttcctcaatagaagtattgctcctttgagtcacatgagagcccacactagcatctggtacatccgccagctttgctatctccggagtcatgagcacaaattctcccatcaagacttgcgaatctgaaaccttcttaccatgactcccatctccacctatttttgctccactagattcttctctcacttgcttgtctctcatagaagaatccttttgttgtggcactggtgccataccccctggcacatttgcatcatcatctccaaaaccaaaatcatccatatccatcggctcgggcttctcctcatttatattctcctccaccctgaattgcagctcatacacaaagtcgccaatggccacatccacatattgtggaatgaggtttggatcaagaactgacacttgtagtcgacatacatcaaacttgttggtgaacttcatatcaactgccttggtcgcacctaaaatcgaacccaccgcccagataagaagaaattgaatcatcacagacggtaaaccaatgaattggacccaaaccttcttaagcttcgcttttgctttttttctgaaacacatattcctcaatttttatatatgcactatgttctgtttgcaccacccccattccaacgtatgatcgagttcagaacgtgacggaaaagtggtcttgaagcaattaaccccagtctaaacaacctcccaattccatttactagaaaccatcctctgcaattctgagatcacttgtgcctcagagagaacaccccttgtaactttaaccacagccgctgctacctcctctttagggtttgttcgcttattttcaatccatatggattgaagtggattgatacggattgagagagattttgacttactagggattgaaaccccctcaatccatatggattggggtagaaccgaacaagcccttaggcttacttttatgctcaaaaggtatgtaataaaatccaagaccatccaaagcataaccacaggtcaaagcacttaatttatccacacatgccctttggttagacatctatagcagtagggttgcttaccagtgccttctttccctcctagcttgtttcactagcttgacatcagaccctccctgatcaggtaccagaacaccttgtggattttagaatagactctaaacaatggaggtgtgggcacagcccctgtaagcgaggaattcaagccgctgaccggcacaggagcttttcccagagcttgatttagcaaggcaatagcctgaacaaacatgtctgttgagggagatgaaccaagcccagcgactgccgacaagtcgacggatttggctccctggagctcacctccacgctgcaggttaccacctaaaaaagacccactttcagaaccaaaccccccacgggtccctccggcagaaccagctccatgaccaggattgaaattctggccaccagcatcattgtcaaaccctcccctgttgccgtggtaggggcgccggaaaccacatctgttcatcaatcctggctgcccattgttatgcccgcctcgattgcctcctgctccatcacatcctccttcatgccccatctgattcctgtcgcctgccatggtgatggcgtctgcgtacgaggaaatgaagggacaaacaacggcagtcgaacgaactatggcctatgggagccctagagaaaccaaccgtgctgtcagttcttgcaaacaaggaaatcagggcatatgtgggccaagctttaagacccggtgccgaaaagaagagtcgctaggccctggactcacagccacagaaacctttccacgacaactttttgaaactctccattaatcctagagccagctgagttgcgatccaccgttctcggtggcacattagcattagcttgctgatattgtggccaagatttggttcttggagaagcaaacttggctctgcttaacgcatcccccaaagatttggttctaccttagcagcaaaataataatgcagtagtataggcttaaggcagtgttttaaaactgggagattcaaaagatcagtggcttgccgttgctcaaaagaactggcggctcgtggagctctttgacgatgcacggcttttcggaacgcggttctagggctctccagtgtaagtgtaacctacgcaccgcaaacaataaaacacaccaattgaggcaaaacaatgtcgagagcgaag

SEQ ID NO: 38 - protein, 639 aa, Zea mays
MNMHQPCFLQYLLLILVPLLLSASAYDRSGEFTFDGFSGNDLITMDGAASVTNGLLSLTSGQKELRGHAFYAFPLDFTSAAVPNSSAVLSFSTTFVFAIIGPYDDLSGHGLAFVLSSTNDLFTALPSQYLGLLNSWNDGNSSNHLLAIELDTIQSTQFNDINDNHIGIDINSLSSVASHTAGYYTSNGEFHPLKLISRKPMQVWVDYDSNHIMLNVTIAPYFMSSTKPSRPLLSTIFNLSSILPTATVYAGFSSATGTLNCKHYVLGWSFKLNGEAASLNYSALSVKTIQEIAQQVPTSDHPGSYKVLICKILLPIVAIFVLVSAVLVKVHLKRRSQERAELDWQREYGPPSFTYKELLAATHGFKDKMLLGRGGFGSVYKGVMPISKQIAAIKRVSPESRQGMREFMAEITILGHLRHRNLVQLIGYCSHKQHLLLVYDYMPNGSLDCYLHTQDHGSTNLCWAQRFHIIKGIASGLFYLHEDWEQVVIHRDIKTSNVLLDSEMNARLGDFGLARSHDHGADAHTTHVAGTYGYIAPELARLGKATKATDIFAFGVLMMEVTCAIRPIWVNTTDGEPLALADWVLAAWQGGSITDAVDPRLDSYVDEEIELVLKLGLLCSHPSPNARPCMRLVMQYLPQ

SEQ ID NO: 39 - DNA, 4132 bp, Zea mays
  source 1..4132: mol_type = other DNA
atggaccggatgctgctcgaccagctggccggcgaggccctgcgggaggtgctgcacgcggtgcagggcaccctgttctgccgctccaccgccgagcgcctgcgccggagcgtcgagccgctgctgccgctcgtccagggcctcggcccgcacagcacccagcgctccgcgggggagctcggcgagctcgcggcgcgggtcagggaggcgctcgacctggcgcgccgcgccgccgcgtccccgcgctggaacgtctaccgcgccgcgcagctgtcgcgccggatggaggcggccgaccgcggcatcgcgcgctggctggagcgccacgcccccgcgcacgtcatcggcggcgtgcgcaggctccgcgacgaggccgacgcgcgcatcggtcgcctcgagcgccgcgtcgaggagatcgccgccgccaccgcgcagccgccgccccccgccctctccgtccccgtcgcgccgccgccgcacaagggcgtgcccatgccgatggaggcgccgctcgctaagcccgccttcgtcgctatgacgaaggaggtgccgcagcacaagggcatggctatgtcggagccggtgccggcgaaggcggcgcccgccaaagccggggtgatggccatggacatcgccgacggacacgaagacgcggaggggatggttggcggcggcgtcaaggtggccaaggaaaaggtgaaggagatggttatgagcggcggcggcagctgggaggtggtcgggatctccggcatgggcggcagcggcaagaccacgctcgccatggaggtcttcagggatcacaaggtccgaggtaagcagaacatcgaccaccagatccaaaccttcttttccttcagttctttccaaatcctgtgcaaaggtcgcgtctttcacaggaatcgtcctgtccttccctgcaaaaattgcatcttccccatgaatagtgggtgctaaattctccaaattggcggcatagcatcgtaggtggttggtttgcgtgcgtgtcgtgcaattaggtaaaggcgaggttgatgctttcgtttttccccaaaccacatgttcggtcaaatttggcgctttgaccaccagctagtgacaactgtactgttgtgatggggtttcagattgcttttgtgaattaccatgcttggacttgagtaaccttatcgtgtcgtgttcatggaccatggtggctacttaatcttaaatcaagatacgtatctgcttaacgaaccgcacatgagactaatcaaatctatttcacatagaataaacccaattcagatccaagtaacctgcttttttgaagaaaaaaaaatctggagctgtgctaattttaggatctcctttcagtgcaaggaattgggatgacgatgttgatttagacctagtttaggtactctagtattgaccataatctatatatattgaggtggattaaggtgtaacttaaactaatttacaccccaatccactttaacacatgtggattgaggtcaataccagaatacccctaatgccgcaacccgggtttgtagctccatcatctctgttttccaccaaaaaactcatagtaaacttaaattcaattgtcacaacaacatatattcaacgattttaagtgactgctatcccaaaccgtagctgatcttagggcatgtacagtggagagacaccaaaacggttctccaagcacaggagacaactaagagactctattgtacagtggagtgtctataaacgtagtctattaataaatacaaaattaaatgtatttgtatagcatcagatcgatagaacagacgacaaattcgtacagtgggaagtgaggcgtctgttgctacttggtttacgagccagaggcgtctcttcacggagagacggctctaagatttttttgcaaataaccccttaaacaccttaagagcctccacattaaacaccactgtacatgtccttaacttcatggtatgctttgtggtcaacatggtgattatggatctcatcaaaagcccatggctacatatctgctccctgtttgcaagctctctctctcccccaccccttttcatgattctgacatagtttctttttttctacagcctacttcaacgacaggatcttcttcgagacgatctcgcagtccgcgaatctggaggccatcaagatgaagctgtgggagcagatcagcggcaacatggtgctgggtacatacaaccagatcccagaatggcagctcaagctaggaccaagggaccgaggacccgtccttgtgatcctcgacgatgtttggtctctcccgcagcttgaggagctcatcttcaagttccctgggtgcaagaccctagtcgtatcaaggttcaagttccccacgctggtgaaacagacgtatgagatgcagctgctagacgaggcggcggctctgtccgtcttctgccgcgctgcgttcgaccaggagtgtgttccgcagaccgccgacaagagattggtcaggcaggtctctgcagagtgcagaggtctccctctggctctgaaggtcatcggcgcgtcgctgcgcgaccagcctccgaagatttggctcagcgccaaaaaccggttgtctcgaggagaggccatttctgactgccatgagaccaagcttctggagaggatggcggccagtgtcgagtgcttgtccgagaaggttagggactgtttccttgacctgggctgcttcccggaggacaagaagatccccctcgacgtcttgatcaacatctggatggagatccatgaccttgatgagccagatgcttttgccatcttggttgagctttcgaacaagaaccttcttaccctcgttaacgatgcacagtacgtatcatcggacatttatgtgcttcaaaatgttcagaacttagatatcccaataacaggtttttctaactctgctgttctatacgtgcaggaacaaggctggagatctgtacagtagctaccatgactactcggtgacacagcacgacgtgttgagagatcttgctcttcacatgagcgggcgtgacccgctcaacaagcgcaggcggttggtgatgccgagaagggaagaaacacttccgagggattggcagaggaacaaggatgctccgtttgaagctcagatagtctccattcatacaggtatagcgttagtagttaattgttcttcattacatttgtagatattcatcgctaacaactcgtcatccaacttatttagtgcgcttattctgaattcctactgaaatttccaactatttccaaaactccaggcgaaatgaaagaatccgactggttccagatgagcttccccaaggcagaagtgctgatcctcaacttcgcgtcgagcctgtactacctgccgccgttcatcgcgacgatgcagaacctgaaggccctggtgctgatcaactacggcagcagcagcagcagcgcagccctggacaacctctccgccttcaccacgctgagcgggctgaggagcctgtggctggagaagatcaggctgccgccgctgcccaagacgacgatcccgctgaggaacctgcacaagatctcgctcgtgctctgcgagctgaacagcagtctaagagggtcgacgatggacctgtcgacgacgttcccgcgcctgtccaacctgacgatcgaccactgcatagacctcaaggagctgccgccgagcgtctgcgagatcgggtccctggagaccatctccatctccaactgccacgacctcaccgagctgccatacgagctggggcggctgcgctgcctcagcatcctccgcgtgtacgcctgcccggcgctgtggcggctgccggcgtcggtgtgcagcctgaagcggctcaagtacctggacatctcgcagtgcatcaacctgacggacctccccgaggagctcggccacctgacgagcctggagaagatcgacatgcgcgagtgctcgcgcctcaggagcctccccaggtcgtcgtcctcgctcaagtccctcggacacgtcgtgtgcgacgaggagacggcgctgctgtggcgtgaggccgagcaggtcatccctgacctccgcgtgcaggtggccgaggagtgctacaacctggactggctcgcggactga

SEQ ID NO: 40 - DNA, 2616 bp, Zea mays; identical to SEQ ID NO: 28
  source 1..2616: mol_type = other DNA

SEQ ID NO: 41 - protein, 871 aa, Zea mays; identical to SEQ ID NO: 29

SEQ ID NO: 42 - DNA, 3639 bp, Zea mays
  source 1..3639: mol_type = other DNA
atggagttggaccggctgctgctcgaccagctggctggcgaggccctacgggagcttctgcacgcggtgcagggcaccctgttctgccgctccaccgccgagcgcctgcgccggagcgtcgagccgctgctgccgctcgtgcagggcctcggcccgcacgcccagcgctccgcgggggacctcggcgagctcgcggcgcgggtcagggaggcgctcgacctggcccgccgcgccgccacgtccccgcgctggaacgtctaccgctccgcgcagctgtcgcgccggatggaggcggccgaccgcggcatcgcgcgctggctggagcgccacgcccccgcgcacgtcatcggcaacgtgcgcgggctccgcgacgagtcccacgcgcgcatcgcccgcctcgagcgccgcgtcgacgagatcgccgccaccgccgcgcagccgccgcccccagccctctccgtccccgtcgcgccgcacaagggcgtggccatgccgatggaggtgccaactcacaagggcatggctatgccgatgccggttccggtgcaggcggtgcccgccaaggccggggtggtggccatggacatggacctcaccgagggacacgaaaacgaggggatggttggcgccggcgttaaggtggccaaggaaaaggtgaaggagatggttatgagcggcggcggcggctgggaggtggtcggtatctccggcatgggcggcagcggcaagaccacgctcgccatggagatcttcagggatcataaggtccgaggtaaggagaacaagaaccagatcataaccatcttttccttcagtccttttccaaatcgtgtgcaaaggtcgcgtctttctcaggaatcgtcccgtcctttcgtgcaaaaattttagcttcccatgaattgttgggtggaaaattctccagattgggggcgtggccgtggcatcttggatggttggtttacgtgcgtgtcgtgcaattaggtgaaggcgaggttgatgctttcgcttttccccaaacctcatgcttggtcaaatttggcgctttgaccagctagtggcaactgtactattgtgatggggttagattacttctgaattctcacaactataagtgtaccacaacatctactttgaaattctgcttggacttgagtaactttatcgtgttcatggttggctagtggctgcttaatcttaaatcaagatagatacctgtttaatgaacggcacatgagactaactaaatctatttcacaaagaatgaacccaattcagatccaactatccaagtaacctgtttcttcggggggaaaaaagaagatctggagctatcccaatttattgtcttctgctagtgcaaggaattaggatcggtgatgttggtttatagcagtaattaagatagctaaattcagcagcttgtgttcatagctccgtcgtctctgcttgccacaaaaaataaaagatcatagtaaacttaaattcaattaccacaacagcatctactggccaattcgaagtgactgctatcccagatcttagttcagtctagcttcatgattgtctttgtgatctgatccctttttgcaagctttttccccccttcaggattgtctttgtgattctgattctgacgtagtttcttcctttctacagcctacttcaatgataggatcttctttgagacgatctcacaatccgcaaacttggaggccatcaagatgaagctgtgggagcagatcagcggcaacatggtgctcggtgcatacaaccagatcccagaatggcagctcaagttaggaccaagagaccgagggcctgtccttgtgatccttgacgatgtttggtctctcccacagctcgaggagctcaccttcaggttccctgggtgcaagactctagttgtgtcgaggttcaagttccccacgctggtaaaacagacatacgaaatgcagttgctagacgaggaggcggccttgtccgtcttctgccgtgccgctttcgatcaggagtgtgttccgcggactgctgacaagagattggtcaggcaggtctctgcagagtgcaggggccttccactggctctgaaggttattggtgcgtcgttgcgcgaccagcctcctaagatctggctcagcgccaagaaccggctgtctcgaggagaggctatttccgactcccatgagaccaagcttctagagaggatggcggcaagcgtcgagtgcttgtcggagaaggttagagactgcttccttgatctgggatgcttcccggaggacaagaagatcccccttgatgtcttgatcaacatctggatggaggttcatgatcttgatgaaccagatgctttcgccatcttggttgagctttcgaacaagaaccttcttaccctcgttaacgatgcacagtatgtatcgtcggtcatttgtgtgcttcaaaatgttctatgtcgcaaataacatggcttttaaactccttttttgctgctctgccttcaggaacaaggctggagatttgtacagcagctaccatgactactcggtgacacaacatgatgtgctgagagatcttgctcttcacatgagtgggcgtgaccctctgaacaagcgtaggcggttggtgatgccgagaaaggaagagacacttccaagggactggcagaggaataaggatactccgtttgaagctcagatagtttccattcatacaggtacagtacagctaccattagtcatttactgaacatgattttgtagctttcttccttggggcagaatgatctgaccaactattccgaaaactccaggtgaaatgaagggatctgactggttccagatgagcttccccaaggcagaagtgctcatcctcaacttcgcctcaagcctatactacctcccgccgttcatcgcgtcgatgcagaacctgaaagccctggtgctgatcaactacggcaccagcagcgcggcccttgacaacctatccgccttcaccacgctgaacggcctgaggagtctctggctggagaagatcaggctcccgccgctgccgaagaccaccatcccgctgaagaacctgcacaagatctcgctcgtcctctgcgagctgaacagcagcctgagagggtcgacgatggacctgtccatgacattcccgcgcctctccaacctcacgatcgaccactgcatagacctaaaggagctgccagcaagcatctgcgagatcggctccctggagaccgtctccatctccaactgccacgacctcaccgagctgccatacgagctgggcaagctgcactgtctgagcatcctccgggtgtacgcctgcccggcgctgtggcggctcccggcgtcggtgtgcagcctgaagaggctcaagtacctcgacatatcccagtgcatcaacctgacggacctcccggaggagctcggccacctgacgagcctggagaagatcgacatgcgggagtgctctcgcctcaggagcctcccgaggtcgtcgtcctccctcaagtccctcggccacgtcgtgtgcgacgaggagacggcgctgctgtggcgggaggccgagcaggtcatccctgacctccgggtgcaggtggccgaagagtgctacaacttggactggctagcggactga

SEQ ID NO: 43 - DNA, 2568 bp, Zea mays
  source 1..2568: mol_type = other DNA
atggagttggaccggctgctgctcgaccagctggctggcgaggccctacgggagcttctgcacgcggtgcagggcaccctgttctgccgctccaccgccgagcgcctgcgccggagcgtcgagccgctgctgccgctcgtgcagggcctcggcccgcacgcccagcgctccgcgggggacctcggcgagctcgcggcgcgggtcagggaggcgctcgacctggcccgccgcgccgccacgtccccgcgctggaacgtctaccgctccgcgcagctgtcgcgccggatggaggcggccgaccgcggcatcgcgcgctggctggagcgccacgcccccgcgcacgtcatcggcaacgtgcgcgggctccgcgacgagtcccacgcgcgcatcgcccgcctcgagcgccgcgtcgacgagatcgccgccaccgccgcgcagccgccgcccccagccctctccgtccccgtcgcgccgcacaagggcgtggccatgccgatggaggtgccaactcacaagggcatggctatgccgatgccggttccggtgcaggcggtgcccgccaaggccggggtggtggccatggacatggacctcaccgagggacacgaaaacgaggggatggttggcgccggcgttaaggtggccaaggaaaaggtgaaggagatggttatgagcggcggcggcggctgggaggtggtcggtatctccggcatgggcggcagcggcaagaccacgctcgccatggagatcttcagggatcataaggtccgagcctacttcaatgataggatcttctttgagacgatctcacaatccgcaaacttggaggccatcaagatgaagctgtgggagcagatcagcggcaacatggtgctcggtgcatacaaccagatcccagaatggcagctcaagttaggaccaagagaccgagggcctgtccttgtgatccttgacgatgtttggtctctcccacagctcgaggagctcaccttcaggttccctgggtgcaagactctagttgtgtcgaggttcaagttccccacgctggtaaaacagacatacgaaatgcagttgctagacgaggaggcggccttgtccgtcttctgccgtgccgctttcgatcaggagtgtgttccgcggactgctgacaagagattggtcaggcaggtctctgcagagtgcaggggccttccactggctctgaaggttattggtgcgtcgttgcgcgaccagcctcctaagatctggctcagcgccaagaaccggctgtctcgaggagaggctatttccgactcccatgagaccaagcttctagagaggatggcggcaagcgtcgagtgcttgtcggagaaggttagagactgcttccttgatctgggatgcttcccggaggacaagaagatcccccttgatgtcttgatcaacatctggatggaggttcatgatcttgatgaaccagatgctttcgccatcttggttgagctttcgaacaagaaccttcttaccctcgttaacgatgcacagaacaaggctggagatttgtacagcagctaccatgactactcggtgacacaacatgatgtgctgagagatcttgctcttcacatgagtgggcgtgaccctctgaacaagcgtaggcggttggtgatgccgagaaaggaagagacacttccaagggactggcagaggaataaggatactccgtttgaagctcagatagtttccattcatacaggtgaaatgaagggatctgactggttccagatgagcttccccaaggcagaagtgctcatcctcaacttcgcctcaagcctatactacctcccgccgttcatcgcgtcgatgcagaacctgaaagccctggtgctgatcaactacggcaccagcagcgcggcccttgacaacctatccgccttcaccacgctgaacggcctgaggagtctctggctggagaagatcaggctcccgccgctgccgaagaccaccatcccgctgaagaacctgcacaagatctcgctcgtcctctgcgagctgaacagcagcctgagagggtcgacgatggacctgtccatgacattcccgcgcctctccaacctcacgatcgaccactgcatagacctaaaggagctgccagcaagcatctgcgagatcggctccctggagaccgtctccatctccaactgccacgacctcaccgagctgccatacgagctgggcaagctgcactgtctgagcatcctccgggtgtacgcctgcccggcgctgtggcggctcccggcgtcggtgtgcagcctgaagaggctcaagtacctcgacatatcccagtgcatcaacctgacggacctcccggaggagctcggccacctgacgagcctggagaagatcgacatgcgggagtgctctcgcctcaggagcctcccgaggtcgtcgtcctccctcaagtccctcggccacgtcgtgtgcgacgaggagacggcgctgctgtggcgggaggccgagcaggtcatccctgacctccgggtgcaggtggccgaagagtgctacaacttggactggctagcggactga

SEQ ID NO: 44 - protein, 855 aa, Zea mays
MELDRLLLDQLAGEALRELLHAVQGTLFCRSTAERLRRSVEPLLPLVQGLGPHAQRSAGDLGELAARVREALDLARRAATSPRWNVYRSAQLSRRMEAADRGIARWLERHAPAHVIGNVRGLRDESHARIARLERRVDEIAATAAQPPPPALSVPVAPHKGVAMPMEVPTHKGMAMPMPVPVQAVPAKAGVVAMDMDLTEGHENEGMVGAGVKVAKEKVKEMVMSGGGGWEVVGISGMGGSGKTTLAMEIFRDHKVRAYFNDRIFFETISQSANLEAIKMKLWEQISGNMVLGAYNQIPEWQLKLGPRDRGPVLVILDDVWSLPQLEELTFRFPGCKTLVVSRFKFPTLVKQTYEMQLLDEEAALSVFCRAAFDQECVPRTADKRLVRQVSAECRGLPLALKVIGASLRDQPPKIWLSAKNRLSRGEAISDSHETKLLERMAASVECLSEKVRDCFLDLGCFPEDKKIPLDVLINIWMEVHDLDEPDAFAILVELSNKNLLTLVNDAQNKAGDLYSSYHDYSVTQHDVLRDLALHMSGRDPLNKRRRLVMPRKEETLPRDWQRNKDTPFEAQIVSIHTGEMKGSDWFQMSFPKAEVLILNFASSLYYLPPFIASMQNLKALVLINYGTSSAALDNLSAFTTLNGLRSLWLEKIRLPPLPKTTIPLKNLHKISLVLCELNSSLRGSTMDLSMTFPRLSNLTIDHCIDLKELPASICEIGSLETVSISNCHDLTELPYELGKLHCLSILRVYACPALWRLPASVCSLKRLKYLDISQCINLTDLPEELGHLTSLEKIDMRECSRLRSLPRSSSSLKSLGHVVCDEETALLWREAEQVIPDLRVQVAEECYNLDWLAD

(19) United States
(12) Patent Application Publication
(10) Pub. No.: US 2025/0171792 A1
(43) Pub. Date: May 29, 2025

(54) MULTIPLE DISEASE RESISTANCE GENES AND GENOMIC STACKS THEREOF

(71) Applicant: PIONEER HI-BRED INTERNATIONAL, INC., JOHNSTON, IA (US)

(72) Inventors: JEFFREY HABBEN, URBANDALE, IA (US); GIRMA M. TABOR, JOHNSTON, IA (US); SHAWN THATCHER, URBANDALE, IA (US)

(73) Assignee: PIONEER HI-BRED INTERNATIONAL, INC., JOHNSTON, IA (US)

(21) Appl. No.: 18/840,335

(22) PCT Filed: Feb. 22, 2023
(86) PCT No.: PCT/US2023/062981

§ 371 Date: Aug. 21, 2024

Related U.S. Application Data
(60) Provisional application No. 63/268,406, filed on Feb. 23, 2022.

(51) Int. Cl. C12N 15/82

(52) U.S. Cl. CPC C12N 15/8213; C12N 15/8261; C12N 15/8274; C12N 15/8286

(57) ABSTRACT

The field is molecular biology, and more specifically, methods for chromosomal engineering of multiple native genes, such as disease resistance genes in a genomic locus using site-specific editing to produce plants. Also described herein are methods of generating heterologous genomic locus in a plant that comprises a plurality of intraspecies polynucleotide sequences.

### FIELD

The field is molecular biology, and more specifically, methods for chromosomal engineering of multiple native genes, such as disease resistance genes in a genomic locus using site-specific editing to produce plants.

### REFERENCE TO SEQUENCE LISTING SUBMITTED ELECTRONICALLY

The official copy of the sequence listing is submitted electronically as an XML formatted sequence listing, with a file name of “108029-US-PCT Sequence Listing” created on Aug. 20, 2024. The file has a size of 162,550 bytes and is filed concurrently with this specification. The sequence listing contained in this XML formatted document is part of the specification and is incorporated herein by reference in its entirety.

### BACKGROUND

Plants contain a variety of genes and allelic variations thereof in their chromosomes. But those genes and alleles are often not linked in a manner to facilitate faster breeding in combination with other traits such as insect resistance and herbicide tolerance. For example, resistance against multiple diseases is an essential component of crop improvement especially as disease pressure and patterns are quickly evolving under a changing climate. Resistance against a specific disease is typically achieved by introgressing a genomic region from a resistant source to an elite line. This process is time consuming and often leads to yield drag and other deleterious effects. In addition, introgressing loci conferring resistance against multiple diseases becomes impractical (in the context of time and resources) because of the number of loci involved and difficult in the case of genetically linked loci. This disclosure provides various methods and compositions to overcome some of these difficulties in breeding with multiple loci and provides a platform for chromosomal engineering of gene stacks, such as for example, disease resistance genes.

### SUMMARY

Limitations of conventional breeding for introgressing a genomic region from a source to an elite line can be overcome by the compositions and methods described herein.

Presented herein are embodiments that describe a method for defining a region of the crop genome specifically engineered to confer disease resistance against multiple diseases, pathogen races, and combinations thereof. Further, disclosed herein is a method for inserting multiple disease resistance genes by gene editing within the defined engineered region. Furthermore, disclosed herein is a method for deploying the engineered region in combination with other traits in a product context, for example, in combination with short stature and/or insect control traits.

In one aspect, disclosed herein is a method for deploying the engineered region in combination with other traits in a product context, for example in combination with insect control trait. Insect control traits include traits conferred by transgenes encoding pesticidal proteins derived from ferns, Bacillus thurigiensis (Bt), or other bacteria such as Pseudomonas sp., Photorhabdus sp., Xenorhabdus sp., Clostridium bifermentans or Paenibacillus popilliae. Other insect control traits include pesticidal traits conferred by transgenes encoding RNA-based insect control agent such as RNA interference or RNA silencing, dsRNA, siRNA, or miRNA.

In some embodiments, an insect control trait can be a pesticidal protein selected from one or more of Cry1B, Cry1D, DvSnf7, Cry75A and/or Vip4Da2. In another aspect, the insect control trait is transgenic corn event MON95379, and the engineered region is deployed within 30 cM, about 20 cM, about 15, about 10, about 5, about 4, about 3, about 2, about 1 cM, about 0.5 cM, about 0.1 cM, or about 0.01 cM of MON95379. In yet another embodiment, the insect control trait is transgenic corn event MON95275, and the engineered region is deployed within 30 cM, about 20 cM, about 15, about 10, about 5, about 4, about 3, about 2, about 1 cM, about 0.5 cM, about 0.1 cM, or about 0.01 cM of MON95275.

In one aspect, disclosed herein is a method for deploying the engineered region in combination with other traits in a product context, for example in combination with transgenic events selected from event designations: MON95379 (US20200032289), MON87403 (US20200087738), MON87460 (US20190376078), MON94100 (US20200123559), MON89034 (U.S. Pat. No. 8,062,840B2), MON87411, MON87751 (U.S. Pat. No. 9,719,145B2), MON 87427, MON 87429, and a combination of the foregoing, for example. In one embodiment, the engineered region is deployed within 30 cM, about 20 cM, about 15, about 10, about 5, about 4, about 3, about 2, about 1 cM, or about 0.5 cM of MON95379, MON87403, MON87460, MON94100, MON89034, MON87411, MON87751, MON87427, and MON87429, for example.

In one aspect, disclosed herein is a method for deploying the engineered region in combination with other traits in a product context, for example in combination with short stature locus selected from one or more of the loci listed in Table 1 below. In certain examples, the engineered region is deployed within 30 cM, about 20 cM, about 15, about 10, about 5, about 4, about 3, about 2, about 1 cM or about 0.5 cM of a locus listed in Table 1 below.

TABLE 1
Short stature loci in corn

| Gene / Mutant | B73 V4 homolog | Chromosome | Position |
|---|---|---|---|
| BR1 | Zm00001d032194 | 1 | 216,643,340 |
| BR2 | Zm00001d031871 | 1 | 204,748,411 |
| D8 | Zm00001d033680 | 1 | 270,918,085 |
| D9 | Zm00001d013465 | 5 | 12,228,329 |
| na1 | Zm00001d042843 | 3 | 181,821,465 |
| na2 | Zm00001d014887 | 5 | 67,026,171 |
| nana2-like1 | Zm00001d051847 | 4 | 172,100,463 |
| d1 | Zm00001d039634 | 3 | 9,745,817 |
| d3 | Zm00001d045563 | 9 | 26,822,040 |
| Dw1 | Zm00001d038988 | 6 | 168,296,702 |
| Dw2-1 | Zm00001d003812 | 2 | 60,529,115 |
| GA20-1 | Zm00001d034898 | 1 | 305,074,531 |
| Dw2-2 | Zm00001d025342 | 10 | 114,817,295 |
| GA20-2 | Zm00001d007894 | 2 | 241,897,454 |

Disclosures relating to genes like BR2, D8, D9, na1, na2, nana2-like1, d1, d3, Dw1, and Dw2-1 have been published. See, e.g., Multani et al. (2003) Science 302:81-88; Shai et al. Plant and Cell Physiology; 2010, 51(11):1854-1868; Hartwig et al. (2011) Proc. Nat'l. Acad. Sci. USA 108(49):19814-19819; Best et al. (2016) Plant Physiol. 171(4):2633-2647; Winkler et al. (1995) Plant Cell 7(8):1307-1317; Yamaguchi et al. (2016) Sci Rep 6:28366; and Hilley et al. (2017) Sci Rep 7:4616. GA20-1 and GA20-2 are similar to rice gibberellin oxidase genes disclosed in Spielmeyer W, et al. (2002) Proc. Nat'l. Acad Sci. USA 99(13):9043-9048. Genome editing of one or more of these genetic loci has been published, for example, US20200199609A1, incorporated herein by reference in its entirety.

Provided are methods for generating a non-native, heterologous genomic locus in a crop plant or plant cell that comprises a plurality of intraspecies polynucleotide sequences. The methods include introducing two or more intraspecies polynucleotide sequences to a predetermined genomic locus in the plant cell, wherein the introducing step does not result in integration of a transgene or a foreign polynucleotide that is not native to the plant; the intraspecies polynucleotides confer one or more disease resistance traits to the plant; at least one or more of the intraspecies polynucleotides are from different chromosome or the intraspecies polynucleotides are not located in the same chromosome in their native configuration compared to the heterologous genomic locus, prior to their integration into the heterologous genomic locus; and the introducing step comprises at least one site-directed genome modification that is not traditional breeding. In one embodiment, the genomic locus is adjacent to a genomic locus that comprises one or more transgenic traits, the transgenic traits comprising a plurality of polynucleotides that are not from the same plant species. In another embodiment, the adjacent transgenic traits comprise one or more traits conferring resistance to one or more insects. In yet another embodiment, the transgenic trait comprises a herbicide tolerance trait.

In one embodiment, the genomic locus is defined by a chromosomal region that is about 1 to about 5 cM or an equivalent physical chromosomal map distance for the crop plant species. In another embodiment, the chromosomal region is about 10 Kb to about 50 Mb. In some aspects, the plant is a corn, soy, canola, cotton, wheat, rice, or sunflower plant.

Also provided are methods of generating a disease super locus in an elite crop plant genome to increase trait introgression efficiency in the elite crop plant, the method comprising introducing a plurality of disease resistance traits at a predetermined genomic locus of the crop plant chromosome by engineering insertion of a plurality of disease resistant genes, genomic translocation of a plurality of disease resistant genes through targeted chromosomal engineering, engineering duplication of one or more disease resistant genes at the genomic locus by targeted genome modification, or a combination of the foregoing. In one embodiment, the disease super locus is present in sufficient proximity to be in linkage disequilibrium with a transgenic trait. For example, the transgenic trait can be selected from the group consisting of insect resistance, herbicide tolerance, and an agronomic trait. In yet another example, the transgenic trait is a pre-existing commercial trait. The disclosed super locus can increase trait introgression efficiency, for example, by reducing the number of backcrosses by at least 50% or by reducing the number of backcrosses by three generations. In another embodiment, trait introgression efficiency can be increased by reducing the number of backcrosses by at least 10%, 20%, 30%, 40%, 50%, 60%, 70%, 80%, 90%, or 100%. In yet another embodiment, the trait introgression efficiency is increased by reducing the backcrosses by at least one, two, three, or four generations.

Also provided are methods for obtaining a plant or plant cell with a modified genomic locus comprising at least two heterologous polynucleotide sequences that confer enhanced disease resistance to at least one plant disease, or at least two traits resulting in resistance to at least one disease through two different modes of action, wherein said at least two polynucleotide sequences are heterologous to the corresponding genomic locus prior to modification and the two polynucleotides are from the same plant species as the plant or plant cell. The methods include introducing a site-specific modification at at least one target site in a genomic locus in a plant cell; introducing at least two heterologous polynucleotide sequences that confer enhanced disease resistance in genomic proximity to the target site; and obtaining the plant cell having a modified genomic locus comprising at least two heterologous polynucleotide sequences that confer enhanced disease resistance. In one embodiment, the modified genomic locus comprises a target site selected from a MON95379 event, MON95275 event, or site disclosed in Table 1, or Table 2. In another embodiment, at least one of the two heterologous polynucleotides further comprises a site-specific modification, for example, a genetic or epigenetic modification. In one embodiment, a heterologous polynucleotide sequence encodes a polypeptide that has at least 90% identity to a polypeptide sequence selected from the group consisting of RppK (SEQ ID NO: 11), Ht1 (SEQ ID NO: 8), NLB18 (SEQ ID NOs: 3 or 5), NLR01 (SEQ ID No: 29), NLR02 (SEQ ID No: 26), RCG1 (SEQ ID Nos: 31), and RCG1b (SEQ ID Nos: 33). Thus, the heterologous polynucleotide sequence can encode a polypeptide that has at least 91%, 92%, 93%, 94%, 95%, 96%, 97%, 98%, 99%, or 100% identity to a polypeptide sequence selected from the group consisting of RppK (SEQ ID NO: 11), Ht1 (SEQ ID NO: 8), NLB18 (SEQ ID NOs: 3 or 5), NLR01 (SEQ ID No: 29), NLR02 (SEQ ID No: 26), RCG1 (SEQ ID Nos: 31), and RCG1b (SEQ ID Nos: 33). In yet another embodiment, the heterologous polynucleotide sequence encodes a polypeptide that has at least 90% identity to a polypeptide sequence selected from the group consisting of PRR03 (SEQ ID No: 36), PRR01 (SEQ ID No: 38), NLR01 (SEQ ID No: 41), and NLR04 (SEQ ID No: 44). Thus, the heterologous polynucleotide sequence can encode a polypeptide sequence that has at least 91%, 92%, 93%, 94%, 95%, 96%, 97%, 98%, 99%, or 100% identity to a polypeptide sequence selected from the group consisting of PRR03 (SEQ ID No: 36), PRR01 (SEQ ID No: 38), NLR01 (SEQ ID No: 41), and NLR04 (SEQ ID No: 44).

Further provided are methods for obtaining a plant or plant cell with a modified genomic locus comprising at least two heterologous polynucleotide sequences that confer enhanced disease resistance to at least one plant disease, or at least two traits resulting in resistance to at least one disease through two different modes of action, wherein said at least two polynucleotide sequences are heterologous to the corresponding genomic locus. In one embodiment, the method comprises introducing a double-strand break or site-specific modification at one or more target sites in a genomic locus in a plant cell; introducing the at least two heterologous polynucleotide sequences that confer enhanced disease resistance; and obtaining a plant cell or plant having a genomic locus comprising at least two polynucleotide sequences that confer enhanced disease resistance. In one embodiment, the plant or plant cell further comprises MON95379 event, MON95275 event, or site disclosed in Table 1, or Table 2 on the same chromosome arm as the modified genomic locus. In another embodiment, a heterologous polynucleotide sequence encodes a polypeptide that has at least 90% identity to a polypeptide sequence selected from the group consisting of RppK (SEQ ID NO: 11), Ht1 (SEQ ID NO: 8), NLB18 (SEQ ID NOs: 3 or 5), NLR01 (SEQ ID No: 29), NLR02 (SEQ ID No: 26), RCG1 (SEQ ID Nos: 31), and RCG1b (SEQ ID Nos: 33). Thus, the heterologous polynucleotide sequence can encode a polypeptide sequence that has at least 91%, 92%, 93%, 94%, 95%, 96%, 97%, 98%, 99%, or 100% identity to a polypeptide sequence selected from the group consisting of RppK (SEQ ID NO: 11), Ht1 (SEQ ID NO: 8), NLB18 (SEQ ID NOs: 3 or 5), NLR01 (SEQ ID No: 29), NLR02 (SEQ ID No: 26), RCG1 (SEQ ID Nos: 31), and RCG1b (SEQ ID Nos: 33). In yet another embodiment, a heterologous polynucleotide sequence encodes a polypeptide that has at least 90% identity to a polypeptide sequence selected from the group consisting of PRR03 (SEQ ID No: 36), PRR01 (SEQ ID No: 38), NLR01 (SEQ ID No: 41), and NLR04 (SEQ ID No: 44). Thus, the heterologous polynucleotide sequence can encode a polypeptide sequence that has at least 91%, 92%, 93%, 94%, 95%, 96%, 97%, 98%, 99%, or 100% identity to a polypeptide sequence selected from the group consisting of PRR03 (SEQ ID No: 36), PRR01 (SEQ ID No: 38), NLR01 (SEQ ID No: 41), and NLR04 (SEQ ID No: 44). In yet another embodiment, the plant or plant cell further comprising MON95379 event, MON95275 event, or site disclosed in Table 1, or Table 2 on the same chromosome arm as the modified genomic locus, wherein the modified genomic locus may be about 30 cM, about 20 cM, about 15, about 10, about 5, about 4, about 3, about 2, about 1 cM, or about 0.5 cM from the MON95379 event, MON95275 event, or site disclosed in Table 1, or Table 2.

Further provided are plants comprising a modified genomic locus, the locus comprising at least a first modified target site and second modified target site, wherein the first modified target site comprises a first polynucleotide sequence that confers enhanced disease resistance to a first plant disease, and wherein the second modified target site comprises a second polynucleotide sequence that confers enhanced disease resistance to the first plant disease or to a second plant disease, wherein the first and the second polynucleotide sequences are heterologous to the modified genomic locus and are present within a genomic window of less than about 1 cM.

Also provided are methods for obtaining a plant or plant cell with a modified genomic locus comprising at least two polynucleotide sequences that confer enhanced disease resistance to at least one plant disease, or at least two traits resulting in resistance to at least one disease through two different modes of action, wherein said at least two polynucleotide sequences are heterologous to the corresponding genomic locus, wherein the genomic locus is located in the distal region of chromosome 1. In one embodiment, the genomic locus is located in the telomeric region of chromosome 1.

Further provided are methods of breeding transgenic and native disease traits at a single locus in a plant comprising inserting at a single locus in a plant a first heterologous polynucleotide sequence that confers enhanced disease resistance to a first plant disease, and second heterologous polynucleotide sequence that confers enhanced disease resistance to the first plant disease or to a second plant disease; inserting at least one heterologous polynucleotide sequence encoding an insecticidal polypeptide, agronomic trait polypeptide, or a herbicide resistance polypeptide at the single locus; crossing the plant with the single locus with a different plant; and obtaining a progeny plant comprising the single locus; and wherein the single locus allows for fewer backcrosses compared to a plant with traits at more than one locus.

Also provided are methods of introgressing or forward breeding multiple disease resistance loci into an elite germplasm, wherein the timeframe for inserting two or more heterologous polynucleotides from different donor plants into the elite line and developing the homozygous resistant lines is shorter. In one embodiment, the methods comprise improving agronomic traits with multiple disease resistance with reduced yield drag from breeding.

Further provided are methods of stacking genetically linked disease resistance genes from multiple sources. In one aspect, provide are modified crop plants comprising at least two, at least three, or at least four disease resistance trait genes stacked in a single genomic locus, wherein the trait stack in a single locus allows for increased breeding efficiency and wherein the trait stack comprises at least two or more non-transgenic native disease resistant traits introduced through genome modification, the native traits comprising polynucleotides from the same crop plant. In one embodiment, the disease resistant trait genes are native traits (i.e., found in plants that have not been genetically engineered).

Further embodiments increase breeding efficiency for stacked traits, wherein the stacked traits are at a single locus and the stacked traits comprise at least two traits resulting in resistance to two different diseases, or at least two traits resulting in resistance to at least one disease through two different modes of action. In some embodiments, the stacked traits further comprise an insect control trait and/or a herbicide resistance trait at the single locus.

Further provided is a modified genomic locus comprising at least three disease resistance genes that provide resistance against Northern Leaf Blight and/or Southern Corn Rust. The three disease resistance genes can include NLB18, Ht1, and RppK, wherein the at least three disease resistance genes are located in the modified genomic locus. In one embodiment, the modified genomic locus is is a maize plant genomic locus. In one embodiment, the modified genomic locus further comprises a PRR03 gene. In another embodiment, the modified plant further comprises at least one gene selected from NLR01, NLR02, RCG1, RCG1b, PRR03, PRR01, NLR01, and NLR04.

### BRIEF DESCRIPTION OF THE DRAWINGS AND SEQUENCE LISTINGS

FIG. 1 shows an example of a breeding stack approach. Variants 1, 2 and 3 are created independently by inserting respectively 3, 2, and 2 genes of interest at three target sites 1, 3 and 6 in a modified genomic locus or super locus. Variant 1 and variant 2 are combined by crossing using standard breeding methods. Recombinants containing both the Variant 1 insertion at target site 1 and the Variant 2 insertion at target site 3 are selected. The new material is further combined with Variant 3 by crossing using standard breeding methods. Recombinants containing the insertions at target sites 1 and 3 and the insertion at target site 6 are selected. The new material includes different plants, each having different combinations of several genes of interest at the super locus.

FIGS. 2A, 2B, and 2C illustrate possible scenarios to create a multi disease resistance stack. FIG. 2A shows a molecular stacking approach, one construct containing one or more genes of interest is used as the repair template to create an insertion of those genes at a target site at the super locus. FIG. 2B shows a breeding stack approach, genes of interest are inserted independently at several target sites and later assembled by breeding crosses to obtain the desired set of genes at the super locus. FIG. 2C shows a successive transformation approach, one construct containing one or more genes of interest is used as the repair template to create an insertion of those genes at a single target site. The material comprising this first insertion is then used as the transformation background for the next insertion, where another set of one or more genes of interest is inserted at the same or another target site at the super locus. This iterative process may be repeated to obtain the desired combination of genes of interest at the super locus. The three scenarios presented here can be used in combination to assemble the desired set of genes of interest at the super locus.

### DESCRIPTION OF THE SEQUENCE LISTING

| SEQ ID | |
|---|---|
| NO | Sequence Description |
| 1 | NLB18 (PH26N) genomic fragment |
| 2 | NLB18 (PH26N) cDNA 1 |
| 3 | NLB18 (PH26N) Protein 1 |
| 4 | NLB18 (PH26N) cDNA 2 |
| 5 | NLB18 (PH26N) Protein 2 |
| 6 | PH4GP Ht1 Genomic Sequence with Native Promoter and |
| | Terminator |
| 7 | PH4GP Ht1 Longer Model CDS Sequence |
| 8 | Translation of PH4GP Ht1 Longer Model CDS Sequence |
| 9 | Rppk Genomic Fragment |
| 10 | Rppk cDNA |
| 11 | Rppk Protein |
| 12 | DSL1-CR1 Guide with PAM |
| 13 | DSL1-CR3 Guide with PAM |
| 14 | DSL1-CR4 Guide with PAM |
| 15 | DSL1-CR5 Guide with PAM |
| 16 | DSL1-CR6 Guide with PAM |
| 17 | DSL1-CR7 Guide with PAM |
| 18 | DSL1-CR9 Guide with PAM |
| 19 | DSL1-CR14 Guide with PAM |
| 20 | DSL1-CR17 Guide with PAM |
| 21 | DSL1-CR18 Guide with PAM |
| 22 | pze-101020971 |
| 23 | pze-101022341 |
| 24 | NLR02 genomic frag |
| 25 | NLR02 CDS |
| 26 | NLR02 Protein |
| 27 | NLR01 genomic frag |
| 28 | NLR01 CDS |
| 29 | NLR01 Protein |
| 30 | Rcg1 CDS |
| 31 | Rcg1 Protein |
| 32 | Rcg1b CDS |
| 33 | Rcg1b Protein |
| 34 | GLS PRR 03 genomic frag |
| 35 | GLS PRR 03 (VAR1) CDS |
| 36 | CHR4 GLS PRR 03 (VAR1) AA |
| 37 | PRR01 (DRL-019.CDS) |
| 38 | PRR01 AA |
| 39 | NLR01_GENOMIC |
| 40 | NLR01_CDS |
| 41 | NLR01_PROTEIN |
| 42 | NLR04_GENOMIC |
| 43 | NLR04_CDS |
| 44 | NLR04_PROTEIN |

### DETAILED DESCRIPTION

It is to be understood that the terminology used herein is for the purpose of describing particular embodiments only, and is not intended to be limiting. As used in this specification and the appended claims, terms in the singular and the singular forms “a”, “an” and “the”, for example, include plural referents unless the content clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules. Unless defined otherwise, all technical and scientific terms used herein have the same meaning as commonly understood by one of ordinary skill in the art to which this disclosure belongs unless clearly indicated otherwise.

Compositions and methods are presented herein to modify the maize genome to produce maize plants that have enhanced resistance diseases including, but not limited to, northern leaf blight, anthracnose stalk rot, grey leaf spot, southern rust, tar spot, Stewart's Bacterial Wilt, Goss's Bacterial Wilt and Blight, Holcus Spot, Bacterial Leaf Blight, Bacterial Stalk Rot, Bacterial Leaf Streak, Bacterial Stripe and Leaf Spot, Chocolate Spot, Kernel Crown Spot, Corn Stunt, Maize Bushy Stunt, Seed Rot, Seedling Blight, and Damping-off, Pythium Root Rot (and Feeder Root Necrosis), Rhizoctonia Crown and Brace Root Rot, Fusarium Root Rot Diseases, Red Root Rot, Southern Corn Leaf Blight, Northern Corn Leaf Blight, Northern Corn Leaf Spot, Rostratum Leaf Spot, Physoderma Brown Spot, Eyespot, Anthracnose Leaf Blight, Gray Leaf Spot, Sorghum Downy Mildew, Java Downy Mildew, Philippine Downy Mildew, Sugarcane Downy Mildew, Rajasthan Downy Mildew, Spontaneum Downy Mildew, Leaf Splitting Downy Mildew, Graminicola Downy Mildew, Crazy Top, Brown Stripe Downy Mildew, Ergot, Common Smut, Head Smut, False Smut, Common Rust, Southern Rust, Tropical Rust, Gibberella Stalk Rot, Diplodia (Stenocarpella) Stalk Rot, Anthracnose Stalk Rot, Charcoal Rot, Fusarium Stalk Rot, Pythium Stalk Rot, Late Wilt, Aspergillus Ear Rot, Diplodia Ear Rot, Fusarium Kernel or Ear Rot, Gibberella Ear Rot or Red Rot, Nigrospora Ear or Cob Rot, Penicillium Ear Rot and Blue Eye, Mycotoxins and Mycotoxicoses, Maize Dwarf Mosaic, Maize Chlorotic Dwarf, Maize Streak, Maize Rough Dwarf, Root-Knot Nematodes, Lesion Nematodes, Sting Nematodes, Needle Nematodes, Stubby-Root Nematodes, Awl Nematodes, Corn Cyst Nematode, Dagger Nematodes, Lance Nematodes, Ring Nematodes, Spiral Nematodes, Stunt Nematodes, disease caused by a parasitic seed plant such as Witchweed, for example.

Compositions and methods are presented herein to insert a DSL on the same chromosome arm as a known or predicted short-stature locus. In some embodiments, provided are compositions and methods to insert DSL within a known or predicted short-stature locus.

The term “allele” refers to one of two or more different nucleotide sequences that occur at a specific locus. Allele can include single nucleotide polymorphism (SNP) as well as larger insertions and deletions (“Indel”).

The term “intraspecies” refers to organisms within the same species. The term “intraspecies polynucleotide sequence” refers to polynucleotide sequence from the same species such as maize DNA for maize crop, soy DNA for soybean crop, for example.

“Backcrossing” refers to the process whereby hybrid progeny are repeatedly crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F1 generation; the term “BC₁” then refers to the second use of the recurrent parent, “BC₂” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides inplanta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

When a gene is introgressed, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite line. This is also sometimes referred to as “yield drag.”

The term “crossed” or “cross” refers to a sexual cross and involved the fusion of two haploid gametes via pollination to produce diploid progeny (e.g., cells, seeds, or plants). The term encompasses both the pollination of one plant by another and selfing (or self-pollination, e.g., when the pollen and ovule are from the same plant).

The term “Disease Super Locus” or “DSL” as used herein generally refers to a modified genomic locus comprising at least two different disease resistant genes targeting (e.g., that contribute to resistance to) at least two different plant diseases, or comprising at least two different disease resistant genes targeting (e.g., that contribute to resistance to) at least one disease through two different modes of action. In one embodiment, the disease resistance genes are within about 0.1, 0.2, 0.3, 0.4, 0.5, 0.6, 0.7, 0.8, 0.9, 1, 2, 3, 4, 5, 6, 7, 8, 9, or 10 cM away from each other. In another embodiment, disease resistance genes are within about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, or about 1000000 bases away from each other. This DSL may be engineered in a manner that facilitates enhanced breeding with co-located transgenic herbicide and/or insect or other agronomic traits.

A “genetic map” is a description of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. However, information can be correlated from one map to another using common markers. One of ordinary skill in the art can use common marker positions to identify positions of markers and other loci of interest on each individual genetic map. The order of loci should not change between maps, although frequently there are small changes in marker orders due to e.g. markers detecting alternate duplicate loci in different populations, differences in statistical approaches used to order the markers, novel mutation or laboratory error.

A “genetic map location” is a location on a genetic map relative to surrounding genetic markers on the same linkage group where a specified marker can be found within a given species.

“Genetic mapping” is the process of defining the linkage relationships of loci through the use of genetic markers, populations segregating for the markers, and standard genetic principles of recombination frequency.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

“Genetic recombination frequency” is the frequency of a crossing over(recombination) between two genetic loci. Recombination frequency can be observed by following the segregation of markers and/or traits following meiosis.

As used herein, the term “haplotype” generally refers to a chromosomal region defined by a genetic characteristic that includes for example, one or more polymorphic molecular markers. In other words, a haplotype is a set of DNA variations, or polymorphisms, that tend to be inherited together. A haplotype can refer to a combination of alleles or to a set of single nucleotide polymorphisms (SNPs) found on the same chromosome or a chromosomal region. A “haplotype window” generally refers to a chromosomal region that is delineated by statistical analyses and often in linkage disequilibrium. The spatial delineation of a haplotype window may change with available marker density and/or other genotyped information density that can differentiate multiple haplotypes.

The term “heterogeneity” is used to indicate that individuals within the group differ in genotype at one or more specific loci.

An “IBM genetic map” can refer to any of following maps: IBM, IBM2, IBM2 neighbors, IBM2 FPC0507, IBM2 2004 neighbors, IBM2 2005 neighbors, IBM2 2005 neighbors frame, IBM2 2008 neighbors, IBM2 2008 neighbors frame, or the latest version on the maizeGDB website. IBM genetic maps are based on a B73×Mo17 population in which the progeny from the initial cross were random-mated for multiple generations prior to constructing recombinant inbred lines for mapping. Newer versions reflect the addition of genetic and BAC mapped loci as well as enhanced map refinement due to the incorporation of information obtained from other genetic maps or physical maps, cleaned date, or the use of new algorithms.

The term “inbred” refers to a line that has been bred for genetic homogeneity.

As used herein, the term “elite germplasm” or “elite plant” refers to any germplasm or plant, respectively, that has resulted from breeding and selection for superior agronomic performance.

The term “indel” refers to an insertion or deletion, wherein one line may be referred to as having an inserted nucleotide or piece of DNA relative to a second line, or the second line may be referred to as having a deleted nucleotide or piece of DNA relative to the first line.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “line” or “strain” is a group of individuals of identical parentage that are generally inbred to some degree and that are generally homozygous and homogeneous at most loci (isogenic or near isogenic). A “subline” refers to an inbred subset of descendants that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F₂; the IBM2 maps consist of multiple meiosis). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Linkage disequilibrium is most commonly assessed using the measure r², which is calculated using the formula described by Hill, W. G. and Robertson, A, Theor. Appl. Genet. 38:226-231(1968). When r²=1, complete LD exists between the two marker loci, meaning that the markers have not been separated by recombination and have the same allele frequency. The r² value will be dependent on the population used. Values for r² above ⅓ indicate sufficiently strong LD to be useful for mapping (Ardlie et al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population.

“Marker assisted selection” (of MAS) is a process by which individual plants are selected based on marker genotypes.

“Marker assisted counter-selection” is a process by which marker genotypes are used to identify plants that will not be selected, allowing them to be removed from a breeding program or planting.

A “marker haplotype” refers to a combination of alleles or haplotypes at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Exserohilum turcicum”, previously referred to as Helminthosporium turcicum, which is also known as Setosphaeria turcica, is the fungal pathogen that induces northern leaf blight infection. The fungal pathogen is also referred to herein as Exserohilum or Et.

The phrase “Gray Leaf Spot” or “GLS” refers to a cereal disease caused by the fungal pathogen Cercospora zeae-maydis, which characteristically produces long, rectangular, grayish-tan leaf lesions which run parallel to the leaf vein.

“Disease resistance” (such as, for example, northern leaf blight resistance) is a characteristic of a plant, wherein the plant avoids, miminimzes, or reduces the disease symptoms that are the outcome of plant-pathogen interactions, such as maize-Exserohilum turcicum interactions. That is, pathogens are prevented from causing plant diseases and the associated disease symptoms, or alternatively, the disease symptoms caused by the pathogen are minimized or lessened.

A “locus” is a position on a chromosome where a gene or marker is located.

“Resistance” is a relative term, indicating that the infected plant produces better plant health or yield of maize than another, similarly treated, more susceptible plant. That is, the conditions cause a reduced decrease in maize survival, growth, and/or yield in a tolerant maize plant, as compared to a susceptible maize plant. One of skill will appreciate that maize plant resistance to northern leaf blight, or the pathogen causing such, can represent a spectrum of more resistant or less resistant phenotypes, and can vary depending on the severity of the infection. However, by simple observation, one of skill can determine the relative resistance or susceptibility of different plants, plant lines or plant families to northern leaf blight, and furthermore, will also recognize the phenotypic gradations of “resistant”. For example, a 1 to 9 visual rating indicating the level of resistance to northern leaf blight can be used. A higher score indicates a higher resistance. The terms “tolerance” and “resistance” are used interchangeably herein.

The resistance may be “newly conferred” or “enhanced”. “Newly conferred” or “enhanced” resistance refers to an increased level of resistance against a particular pathogen, a wide spectrum of pathogens, or an infection caused by the pathogen(s). An increased level of resistance against a particular fungal pathogen, such as Et, for example, constitutes “enhanced” or improved fungal resistance. The embodiments may enhance or improve fungal plant pathogen resistance.

In some embodiments, gene editing may be facilitated through the induction of a double-stranded break (a “DSB”) in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), and the like. In some embodiments, the introduction of a DSB can be combined with the introduction of a polynucleotide modification template.

A polynucleotide modification template may be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template may be introduced into a cell as a single stranded polynucleotide molecule, a double stranded polynucleotide molecule, or as part of a circular DNA (vector DNA). The polynucleotide modification template may also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous homologous recombination HR machinery is expected to be highly diminished (Mali et al. 2013 Nature Methods Vol. 10. 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence, and the alteration is by deliberate human intervention. Such “alterations” include, for example: (i) replacement of at least one nucleotide, (ii) a deletion of at least one nucleotide, (iii) an insertion of at least one nucleotide, or (iv) any combination of (i)-(iii). An “edited cell” or an “edited plant cell” refers to a cell containing at least one alteration in the genomic sequence when compared to a control cell or plant cell that does not include such alteration in the genomic sequence.

The term “polynucleotide modification template” or “modification template” as used herein refers to a polynucleotide that comprises at least one nucleotide modification when compared to the target nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSBs and modification templates generally comprises: providing to a host cell a DSB-inducing agent, or a nucleic acid encoding a DSB-inducing agent, that recognizes a target sequence in the chromosomal sequence, and wherein the DSB-inducing agent is able to induce a DSB in the genomic sequence; and providing at least one polynucleotide modification template comprising at least one nucleotide alteration when compared to the nucleotide sequence to be edited. The endonuclease may be provided to a cell by any method known in the art, for example, but not limited to transient introduction methods, transfection, microinjection, and/or topical application or indirectly via recombination constructs. The endonuclease may be provided as a protein or as a guided polynucleotide complex directly to a cell or indirectly via recombination constructs. The endonuclease may be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433.

As used herein, a “genomic region” refers to a segment of a chromosome in the genome of a cell. In one embodiment, a genomic region includes a segment of a chromosome in the genome of a cell that is present on either side of the target site or, alternatively, also comprises a portion of the target site. The genomic region may comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

A “modified plant” refers to any plant that has a heterologous polynucleotide purposefully inserted into its genome, wherein the inserted polynucleotide is heterologous to the plant, heterologous to the position in the genome, or has an altered sequence compared to an unmodified plant from the same genetic background. An example of a modified plant is a plant comprising a modified genomic locus or disease super locus disclosed herein, e.g. a modified genomic locus that comprises at least two heterologous polynucleotide sequences that confer enhanced disease resistance to at least one plant disease, or at least two traits resulting in resistance to at least one disease through two different modes of action. Modified plants may be created through transgenic applications, genomic modifications including CRISPR or Talens, traditional breeding, or any combination thereof.

The term “site of action” generally refers to a specific physical location or biochemical site within the organism where a specific ligand or polypeptide acts or directly interacts. For example, an effector polypeptide may interact with a disease resistance polypeptide.

The term “mode of action” generally describes a functional or anatomical change resulting from the exposure of an organism to a substance such as polypeptide or regulatory RNA. The term “mode of action” may also refer to a specific mechanism of recognition or action at the cellular or molecular level.

In some embodiments, a modified plant comprises a heterologous polynucleotide, the transcript of which is alternatively spliced into two messenger RNAs encoding two polypeptides, wherein the two polypeptides have a different site of action or mode of action. In some embodiments, the modified plant has increased resistance durability to a plant pathogen when expressing said transcript, which is alternatively spliced into two messenger RNAs encoding two polypeptides, wherein the two polypeptides have a different site of action or mode of action. In other embodiments, the modified plant has increased resistance to more than one plant pathogen when expressing said transcript, which is alternatively spliced into two messenger RNAs encoding two polypeptides, wherein the two polypeptides have a different site of action or mode of action.

In another embodiment, a modified plant comprises at least two heterologous polynucleotides wherein the polynucleotides produce one or more non-coding transcripts or encode one or more polypeptides. For example, said one or more non-coding transcripts or one or more polypeptides target the same plant pathogen. In another example, said one or more non-coding transcripts or one or more polypeptides target the same plant pathogen via different modes of action.

In one embodiment, a modified plant comprises at least two heterologous polynucleotides wherein the polynucleotides produce one or more non-coding transcripts or encode one or more polypeptides. For example, said least two heterologous polynucleotides are derived from the same species. In another example, said least two heterologous polynucleotides are derived from different species.

TAL effector nucleases (TALEN) are a class of sequence-specific nucleases that can be used to make double-strand breaks at specific target sequences in the genome of a plant or other organism. (See Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein, or complex of proteins, encoded by a Cas gene. A Cas endonuclease as disclosed herein, when in complex with a suitable polynucleotide component, is capable of recognizing, binding to, and optionally nicking or cleaving all or part of a specific DNA target sequence. A Cas endonuclease as described herein comprises one or more nuclease domains. Cas endonucleases of the disclosure includes those having a HNH or HNH-like nuclease domain and/or a RuvC or RuvC-like nuclease domain. A Cas endonuclease of the disclosure may include a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, and US 2015-0059010 A1, both hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein, or a variant thereof, retaining some or all activity in each endonuclease domain of the Cas protein, is a suitable example of a Cas endonuclease that can cleave both strands of a DNA target sequence. A Cas9 protein comprising functional RuvC and HNH nuclease domains is an example of a Cas protein that can cleave both strands of a DNA target sequence. A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in U.S. Patent Appl. Publ. No. 2014/0189896, which is incorporated herein by reference.

A pair of Cas9 nickases may be used to increase the specificity of DNA targeting. In general, this can be done by providing two Cas9 nickases that, by virtue of being associated with RNA components with different guide sequences, target and nick nearby DNA sequences on opposite strands in the region for desired targeting. Such nearby cleavage of each DNA strand creates a double strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, 10, 15, 20, 30, 40, 50, 60, 70, 80, 90, or 100 (or any integer between 5 and 100) bases apart from each other, for example. One or two Cas9 nickase proteins herein can be used in a Cas9 nickase pair. For example, a Cas9 nickase with a mutant RuvC domain, but functioning HNH domain (i.e., Cas9 HNH+/RuvC−), could be used (e.g., Streptococcus pyogenes Cas9 HNH+/RuvC−). Each Cas9 nickase (e.g., Cas9 HNH+/RuvC−) would be directed to specific DNA sites nearby each other (up to 100 base pairs apart) by using suitable RNA components herein with guide RNA sequences targeting each nickase to each specific DNA site.

A Cas protein may be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16. See PCT patent applications PCT/US16/32073, filed May 12, 2016 and PCT/US16/32028 filed May 12, 2016 (both applications incorporated herein by reference) for more examples of Cas proteins.

A guide polynucleotide/Cas endonuclease complex in certain embodiments may bind to a DNA target site sequence, but does not cleave any strand at the target site sequence. Such a complex may comprise a Cas protein in which all of its nuclease domains are mutant, dysfunctional. For example, a Cas9 protein herein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein). In other aspects, an inactivated Cas protein may be fused with another protein having endonuclease activity, such as a Fok I endonuclease.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

The Cas endonuclease can comprise a modified form of the Cas9 polypeptide. The modified form of the Cas9 polypeptide can include an amino acid change (e.g., deletion, insertion, or substitution) that reduces the naturally-occurring nuclease activity of the Cas9 protein. For example, in some instances, the modified form of the Cas9 protein has less than 50%, less than 40%, less than 30%, less than 20%, less than 10%, less than 5%, or less than 1% of the nuclease activity of the corresponding wild-type Cas9 polypeptide (US patent application US20140068797 A1). In some cases, the modified form of the Cas9 polypeptide has no substantial nuclease activity and is referred to as catalytically “inactivated Cas9” or “deactivated cas9 (dCas9).” Catalytically inactivated Cas9 variants include Cas9 variants that contain mutations in the HNH and RuvC nuclease domains. These catalytically inactivated Cas9 variants are capable of interacting with sgRNA and binding to the target site in vivo but cannot cleave either strand of the target DNA.

A catalytically inactive Cas9 can be fused to a heterologous sequence (US patent application US20140068797 A1). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a FokI nuclease to generate double strand breaks (Guilinger et al. Nature Biotechnology, volume 32, number 6, June 2014).

The terms “functional fragment”, “fragment that is functionally equivalent” and “functionally equivalent fragment” of a Cas endonuclease are used interchangeably herein, and refer to a portion or subsequence of the Cas endonuclease sequence of the present disclosure in which the ability to recognize, bind to, and optionally nick or cleave (introduce a single or double strand break in) the target site is retained.

The terms “functional variant”, “Variant that is functionally equivalent” and “functionally equivalent variant” of a Cas endonuclease are used interchangeably herein, and refer to a variant of the Cas endonuclease of the present disclosure in which the ability to recognize, bind to, and optionally nick or cleave (introduce a single or double strand break in) the target site is retained. Fragments and variants can be obtained via methods such as site-directed mutagenesis and synthetic construction.

Any guided endonuclease (e.g., guided CRISPR-Cas systems) can be used in the methods disclosed herein. Such endonucleases include, but are not limited to Cas9, Cas12f and their variants (see SEQ ID NO: 37 of U.S. Pat. No. 10,934,536, incorporated herein by reference in its entirety) and Cpf1 endonucleases. Many endonucleases have been described to date that can recognize specific PAM sequences (see for example—Jinek et al. (2012) Science 337 p 816-821, PCT patent applications PCT/US16/32073, and PCT/US16/32028 and Zetsche B et al. 2015. Cell 163, 1013) and cleave the target DNA at a specific positions. It is understood that based on the methods and embodiments described herein utilizing a guided Cas system one can now tailor these methods such that they can utilize any guided endonuclease system. Various chromosomal engineering tools and methods are illustrated in PCT/US2021/034704, filed May 28, 2021 and the contents thereof are incorporated herein by reference to the extent they relate to certain targeted chromosome engineering applications.

As used herein, the term “guide polynucleotide”, relates to a polynucleotide sequence that can form a complex with a Cas endonuclease and enables the Cas endonuclease to recognize, bind to, and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (See also U.S. Patent Application US 2015-0082478 A1, and US 2015-0059010 A1, both hereby incorporated in its entirety by reference).

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA- combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. The size of the fragment of the cRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA.

The tracrRNA (trans-activating CRISPR RNA) contains, in the 5′-to-3′ direction, (i) a sequence that anneals with the repeat region of CRISPR type II crRNA and (ii) a stem loop-containing portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) into the target site. (See also U.S. Patent Application US 2015-0082478 A1, published on Mar. 19, 2015 and US 2015-0059010 A1, both hereby incorporated in its entirety by reference.)

The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, and US 2015-0059010 A1, both hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 2015-0059010 A1, incorporated in its entirety by reference herein), or any combination thereof.

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “Variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, and US 2015-0059010 A1, both hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence including, but not limited to, a nucleotide sequence within a chromosome, an episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long. The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting may be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein may be performed in such a way that two or more DNA target sites are targeted in the method, for example. Such a method can optionally be characterized as a multiplex method. Two, three, four, five, six, seven, eight, nine, ten, or more target sites may be targeted at the same time in certain embodiments. A multiplex method is typically performed by a targeting method herein in which multiple different RNA components are provided, each designed to guide a guide polynucleotide/Cas endonuclease complex to a unique DNA target site.

The terms “knock-out”, “gene knock-out” and “genetic knock-out” are used interchangeably herein. A knock-out as used herein represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site. In a separate embodiment, a “knock out” may be the result of downregulation of a gene through RNA interference. In some aspects, a double stranded RNA (dsRNA) molecule(s) may be employed in the disclosed methods and compositions to mediate the reduction of expression of a target sequence, for example, by mediating RNA interference “RNAi” or gene silencing in a sequence-specific manner. In some embodiments, a native susceptible copy allele of a gene that has a resistant gene counterpart in the DSL is knocked out by RNA interference or gene editing.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, and WO2015/026886 A1, both hereby incorporated in its entirety by reference.)

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins include, but are not limited to, a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct may further comprise a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. To determine the percent identity of two amino acid sequences or of two nucleic acid sequences, the sequences are aligned for optimal comparison purposes. The percent identity between the two sequences is a function of the number of identical positions shared by the sequences (e.g., percent identity of query sequence=number of identical positions between query and subject sequences/total number of positions of query sequence (e.g., overlapping positions)×100).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombinations: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986)Proc. Natl. Acad. Sci. USA 83:5199-203; Liskay et al., (1987) Genetics 115:161-7.

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. (2014) PNAS (0027-8424), 111 (10), p. E924-E932).

Alteration of the genome of a plant cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., (Scientific American Books distributed by WH Freeman & Co.).

In some embodiments, methods and compositions are provided for inverting large segments of a chromosome, deleting segments of chromosomes, and relocating segments or genes using CRISPR-Cas technology (U.S. Patent Application 63/301,822 filed 29 May 2020).

In some aspects, a DSL chromosomal segment may be moved or otherwise altered using chromosomal rearrangement.
In another embodiment, a chromosomal segment may be rearranged into a DSL. In some aspects, a chromosomal segment is at least about 1 kb, between 1 kb and 10 kb, at least about 10 kb, between 10 kb and 20 kb, at least about 20 kb, between 20 kb and 30 kb, at least about 30 kb, between 30 kb and 40 kb, at least about 40 kb, between 40 kb and 50 kb, at least about 50 kb, between 50 kb and 60 kb, at least about 60 kb, between 60 kb and 70 kb, at least about 70 kb, between 70 kb and 80 kb, at least about 80 kb, between 80 kb and 90 kb, at least about 90 kb, between 90 kb and 100 kb, or greater than 100 kb. In some aspects, the segment is at least about 100 kb, between 100 kb and 150 kb, at least about 150 kb, between 150 kb and 200 kb, at least about 200 kb, between 200 kb and 250 kb, at least about 250 kb, between 250 kb and 300 kb, at least about 300 kb, between 300 kb and 350 kb, at least about 350 kb, between 350 kb and 400 kb, at least about 400 kb, between 400 kb and 450 kb, at least about 450 kb, between 450 kb and 500 kb, at least about 500 kb, between 500 kb and 550 kb, at least about 550 kb, between 550 kb and 600 kb, at least about 600 kb, between 600 kb and 650 kb, at least about 650 kb, between 650 kb and 700 kb, at least about 700 kb, between 700 kb and 750 kb, at least about 750 kb, between 750 kb and 800 kb, at least about 800 kb, between 800 kb and 850 kb, at least about 850 kb, between 850 kb and 900 kb, at least about 900 kb, between 900 kb and 950 kb, at least about 950 kb, between 950 kb and 1000 kb, at least about 1000 kb, between 1000 kb and 1050 kb, at least about 1050 kb, between 1050 kb and 1100 kb, or greater than 1100 kb. In some aspects, the segment is at least about 1 Mb, between 1 Mb and 10 Mb, at least about 10 Mb, between 10 Mb and 20 Mb, at least about 20 Mb, between 20 Mb and 30 Mb, at least about 30 Mb, between 30 Mb and 40 Mb, at least about 40 Mb, between 40 Mb and 50 Mb, at least about 50 Mb, between 50 Mb and 60 Mb, at least about 60 Mb, between 60 Mb and 70 Mb, at least about 70 Mb, between 70 Mb and 80 Mb, at least about 80 Mb, between 80 Mb and 90 Mb, at least about 90 Mb, between 90 Mb and 100 Mb, or greater than 100 Mb.

Error-prone DNA repair mechanisms can produce mutations at double-strand break sites. The Non-Homologous-End-Joining (NHEJ) pathways are the most common repair mechanism to bring the broken ends together (Bleuyard et al., (2006) DNA Repair 5:1-12). The structural integrity of chromosomes is typically preserved by the repair, but deletions, insertions, or other rearrangements are possible. The two ends of one double-strand break are the most prevalent substrates of NHEJ (Kirik et al., (2000) EMBO J 19:5562-6), however if two different double-strand breaks occur, the free ends from different breaks can be ligated and result in chromosomal deletions (Siebert and Puchta, (2002) Plant Cell 14:1121-31), or chromosomal translocations between different chromosomes (Pacher et al., (2007) Genetics 175:21-9).

The donor DNA may be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Further uses for guide RNA/Cas endonuclease systems have been described (See U.S. Patent Application US 2015-0082478 A1, WO2015/026886 A1, US 2015-0059010 A1, US application US 2017/0306349 A1, and U.S. application 62/036,652, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Polynucleotides of interest and/or traits can be stacked together in a complex trait locus as described in US 2013/0263324-A1 and in PCT/US13/22891, both applications hereby incorporated by reference.

In some embodiments, a maize plant cell comprises a genomic locus with at least one nucleotide sequence that confers enhanced resistance to northern leaf blight and a at least one different plant disease are provided herein. Further plant diseases may include, but are not limited to, grey leaf spot, southern corn rust, and anthracnose stalk rot. The disclosed methods include introducing a double-strand break at one or more target sites in a genomic locus in a maize plant cell; introducing one or more nucleotide sequences that confer enhanced resistance to more than one plant disease, wherein each is flanked by 300-500 bp of nucleotide sequences 5′ or 3′ of the corresponding target sites; and obtaining a maize plant cell having a genomic locus comprising one or more nucleotide sequences that confer enhanced resistance to more than one plant disease. The double-strand break may be induced by a nuclease such as but not limited to a TALEN, a meganuclease, a zinc finger nuclease, or a CRISPR-associated nuclease. The method may further comprise growing a maize plant from the maize plant cell having the genomic locus comprising the at least one nucleotide sequence that confers enhanced resistance to northern leaf blight, and the maize plant may exhibit enhanced resistance to northern leaf blight.

A maize plants exhibits enhanced resistance when compared to equivalent plants lacking the nucleotide sequences conferring enhanced resistance at the genomic locus of interest. “Equivalent” means that the plants are genetically similar with the exception of the genomic locus of interest.

In some aspects, the one or more nucleotide sequences that confers enhanced disease resistance include any of the following: RppK (Genomic DNA SEQ ID NO: 9; cDNA SEQ ID NO: 10; Protein SEQ ID NO: 11), Ht1 (Genomic DNA SEQ ID NO: 6; cDNA SEQ ID NO: 7; Protein SEQ ID NO: 8), NLB18 (Genomic DNA SEQ ID NO: 1; cDNA SEQ ID NO: 2 or 4; Protein SEQ ID NO: 3 or 5), NLR01 (Genomic DNA SEQ ID No: 27; cDNA SEQ ID NO: 28; Protein SEQ ID No: 29), NLR02 (Genomic DNA SEQ ID Nos: 24; cDNA SEQ ID NO: 25; Protein SEQ ID No: 26), RCG1 (cDNA SEQ ID Nos: 30; Protein SEQ ID No: 31), RCG1b (cDNA SEQ ID Nos: 32; Protein SEQ ID No: 33), PRR03 (Genomic DNA SEQ ID Nos: 34; cDNA SEQ ID NO: 35; Protein SEQ ID No: 36), PRR01 (cDNA SEQ ID NO: 37; Protein SEQ ID No: 38), NLR01 (Genomic DNA SEQ ID Nos: 39; cDNA SEQ ID NO: 40; Protein SEQ ID No: 41), or NLR04 (Genomic DNA SEQ ID Nos: 42; cDNA SEQ ID NO: 43; Protein SEQ ID No: 44), for example.

As used herein a “complex transgenic trait locus” (plural: “complex transgenic trait loci”) is a chromosomal segment within a genomic region of interest that comprises at least two altered target sequences that are genetically linked to each other and can also comprise one or more polynucleotides of interest as described hereinbelow. Each of the altered target sequences in the complex transgenic trait locus originates from a corresponding target sequence that was altered, for example, by a mechanism involving a double-strand break within the target sequence that was induced by a double-strand break-inducing agent of the invention. In certain embodiments of the invention, the altered target sequences comprise a transgene.

CTL1 exists on Maize Chromosome 1 in a window of approximately 5 cM (U.S. Pat. No. 10,030,245, US Patent Publication No. 2018/0258438A1, US Patent Publication No. 2018/0230476A1). The first maize genomic window that was identified for development of a Complex Trait Locus (CTL) spans from ZM01: 12987435 (flanked by public SNP marker SYN12545) to Zm01:15512479 (flanked by public SNP marker SYN20196) on chromosome 1. Table 2 shows the physical and genetic map position (if available) for a multitude of maize SNP markers (Ganal, M. et al, A Large Maize (Zea mays L.) SNP Genotyping Array: Development and Germplasm Genotyping, and Genetic Mapping to Compare with the B73 Reference Genome. PloS one, Dec. 8, 2011DOI: 10.1371) and Cas endonuclease target sites (31 sites) within the genomic window of interest on the maize chromosome 1.

TABLE 2
Genomic Window comprising a Complex Trait
Locus (CTL1) on Chromosome 1 of maize

| | Cas | | |
|---|---|---|---|
| Name of public | endonuclease | | |
| SNP markers (*) | target or SNP | | |
| or Cas | marker | Physical | Genetic |
| endonuclease | sequence (SEQ | position (PUB | Position (PUB |
| target site | ID NO:) | B73v3) | B73v3) |
| SYN12545* | 1 | 12987435 | 36.9 |
| SYN12536* | 2 | 12988556 | 36.9 |
| 49-CR2 | 3 | 13488227 | |
| 50-CR1 | 4 | 13554078 | |
| 51-CR1 | 5 | 13676343 | |
| SYN14645* | 6 | 13685871 | 37.4 |
| 41-CR2 | 7 | 13830316 | |
| 72-CR1 | 8 | 13841735 | |
| 71-CR1 | 9 | 13846794 | |
| 81-CR1 | 10 | 13967499 | |
| 73-CR1 | 11 | 13986903 | |
| PZE-101023852* | 12 | 14030843 | 37.6 |
| 14-CR4 | 13 | 14038610 | |
| 74-CR1 | 14 | 14089937 | |
| 75-CR1 | 15 | 14226763 | |
| 84-CR1 | 16 | 14233410 | |
| 76-CR1 | 17 | 14245535 | |
| 77-CR1 | 18 | 14344614 | |
| 78-CR1 | 19 | 14380330 | |
| PZE-101024424* | 20 | 14506833 | 37.8 |
| 79-CR1 | 21 | 14577827 | |
| 85-CR1 | 22 | 14811592 | |
| 19-CR1 | 23 | 14816379 | |
| SYN25022* | 24 | 14851517 | 37.8 |
| 86-CR1 | 25 | 14951113 | |
| 08-CR1 | 26 | 14955364 | |
| 43-CR1 | 27 | 15006039 | |
| 11-CR1 | 28 | 15066942 | |
| SYN31156* | 29 | 15070918 | 39.9 |
| 47-CR2 | 30 | 15081190 | |
| 80-CR1 | 31 | 15084949 | |
| 52-CR2 | 32 | 15088711 | |
| 87-CR1 | 33 | 15158706 | |
| 88-CR1 | 34 | 15162366 | |
| SYN31166* | 35 | 15169575 | 40.9 |
| 45-CR1 | 36 | 15177228 | |
| 10-CR3 | 37 | 15274433 | |
| 44-CR2 | 38 | 15317833 | |
| 46-CR2 | 39 | 15345674 | |
| SYN22238* | 40 | 15491134 | 41.7 |
| SYN20196* | 41 | 15512479 | 41.9 |

Provided herein is a modified genomic locus that comprises Disease Super Locus 1 (DSL1). In another embodiment, Disease Super Locus 1 (DSL1) is located in the distal region of chromosome 1 approximately 0.5, 1, 2, 3, 4, 5, 6, 7, 8, 9, or 10 cM away from Complex Trait Locus 1 (CTL1). In one embodiment, a Disease Super Locus (DSL) is located approximately 0.1, 0.2, 0.3, 0.4, 0.5, 0.6, 0.7, 0.8, 0.9, 1, 2, 3, 4, 5, 6, 7, 8, 9, or 10 cM away from at least one different trait locus. In another embodiment, a DSL is located in the telomeric region. In a preferred embodiment, DSL1 is distal to CTL1 within about 0.5 cM to about 5 cM. In yet another embodiment. DSL1 is flanked by pze-101020971 (SEQ ID NO: 22) and pze-101022341 (SEQ ID NO: 23). In some embodiments, CTL1 comprises an insect control trait and a herbicide tolerance trait.

In one aspect, the genomic locus that confers enhanced resistance to northern leaf blight comprises DSL1.

The guide polynucleotide/Cas9 endonuclease system as described herein provides for an efficient system to generate double strand breaks and allows for traits to be stacked in a complex trait locus. Thus, in one aspect, Cas9 endonuclease is used as the DSB-inducing agent, and one or more guide RNAs are used to target the Cas9 to sites in the DSL1 locus.

The maize plants generated by the methods described herein may provide durable and broad spectrum disease resistance and may assist in breeding of disease resistant maize plants. For instance, because the nucleotide sequences that confer enhanced disease resistance in tight linkage with one another (at one locus), this reduces the number of specific loci that require trait introgression through backcrossing and minimizes linkage drag from non-elite resistant donors. In one embodiment, a DSL is located within at least 1 cM, 2 cM, 3 cM, 4 cM, 5 cM, 6 cM, 7 cM, 8 cM, 9 cM, 10 cM, 15 cM, or 20 cM from a QTL for yield stability or disease resistance.

In some embodiments, the maize plants that comprise DSL may be treated with insecticide, fungicide, or biologicals. In one embodiment, the maize plants generated by the methods described herein may require lower levels or fewer number of treatments of fungicide, or biologicals compared to the levels of fungicide, or biologicals required in maize plants that do not comprise DSL. In a further embodiment, the lower levels or fewer number of treatments of fungicide, or biologicals compared to the levels of fungicide, or biologicals required in maize plants that do not comprise DSL may increase the durability of the fungicide, or biologicals.

In one embodiment, the fungicide comprises a fungicide composition selected from the group consisting of azoxystrobin, thiabendazole, fludioxonil, metalaxyl, tebuconazole, prothioconazole, ipconazole, penflufen, and sedaxane. Compositions disclosed herein may comprise fungicides which may include, but are not limited to, the respiration inhibitors, such as azoxystrobin, which target complex III of mitochondrial electron transport; tubulin inhibitors, such as thiabendazole, which bind to beta-tubulin; the osmotic stress related-kinase inhibitor fludioxonil; an RNA polymerase inhibitor of Oomycetes, a group of fungal-like organisms, such as metalaxyl; inhibitors of sterol biosynthesis, which include inhibitors of the C-14 demethylase of the sterol biosynthesis pathway (commonly referred to as demethylase inhibitors or DMIs), such as tebuconazole, prothioconazole, and ipconazole; a respiration inhibitor which targets complex II mitochondrial electron transport, such as a penflufen; a respiration inhibitor which targets complex 11 mitochondrial electron transport, such as sedaxane. Other classes of fungicides with different or similar modes of action can be found at frac.info/docs/default-source/putblications/frac-code-list/frac-code-list-2016.pdf?sfvrsn=2 (which can be accessed on the world-wide web using the “www” prefix; See Hirooka and Ishii (2013), Journal of General Plant Pathology). A fungicide may comprise all or any combination of different classes of fungicides as described herein. In certain embodiments, a composition disclosed herein comprises azoxystrobin, thiabendazole, fludioxonil, and metalaxyl. In another embodiment, a composition disclosed herein comprises a tebuconazole. In another embodiment, a composition disclosed herein comprises prothioconazole, metalaxyl, and penflufen. In another embodiment, a composition disclosed herein comprises ipconazole and metalaxyl. In another embodiment, a composition disclosed herein comprises sedaxane. As used herein, a composition may be a liquid, a heterogeneous mixture, a homogeneous mixture, a powder, a solution, a dispersion or any combination thereof. In another embodiment, a biocontrol agent may be used in combination with a DSL.

Another strategy to reduce the need for refuge is the pyramiding of traits with different modes of action against a target pest. For example, Bt toxins that have different modes of action pyramided in one transgenic plant are able to have reduced refuge requirements due to reduced resistance risk. The same may be done for disease resistance and trait durability. In some aspects, two genes targeting the same disease can increase each trait's durability. For example, the combination of NLB18 and Ht1 (SEQ ID NOs: 3 and 8 respectively) expressed in a plant increase the durability of each trait to increase resistance to northern leaf blight. Accordingly, the combination of NLB18 and Ht1 can be inserted into the same modified genomic locus or DSL. Different modes of action in a pyramid combination also extends the durability of each trait, as resistance is slower to develop to each trait.

In one embodiment, a first Disease Super Locus is stacked with a second Disease Super Locus. In another embodiment, a breeding stack approach is used to obtain a maize plant comprising a first Disease Super Locus stacked with a second Disease Super Locus. In some embodiments, the second Disease Super Locus has at least one different disease resistance gene from the first Disease Super Locus.

In one embodiment, the polynucleotide sequence encoding a disease resistance gene comprises a heterologous promoter. In another embodiment, the polynucleotide sequence encoding a disease resistance gene comprises a cDNA sequence. In yet another embodiment, polynucleotide sequence encoding a disease resistance gene comprises an endogenous disease resistance locus and further comprises a heterologous expression modulating element (EME).

In one embodiment, DSL comprises a polynucleotide that produces a non-coding transcript or non-coding RNA. In another embodiment, the source of non-coding transcripts could be from non-coding genes, or it could be from repetitive sequences like transposons or retrotransposons. In another embodiment, the non-coding transcripts could be produced by RNAi constructs with a hairpin design. In another embodiment, a DSL may comprise one or more polynucleotide sequence that don't encode a polypeptide, but comprise a transposon or repetitive sequence, or a sequence that is transcribed into non-coding transcripts of various sizes such as long non-coding RNAs (lncRNAs), for example. In one embodiment, a non-coding transcript may be processed into small RNAs such as microRNA (miRNA), short-interfering RNA (siRNA), trans-acting siRNA (tasiRNA), and phased siRNA (phasiRNA). In one embodiment, the non-coding genes and sequences in a DSL may share nucleotide sequence homology to specific sequences in plant pathogens or pests, such as viruses, bacteria, oomycetes, fungus, insects, and parasitic plants. A non-coding transcript or processed products such as small RNAs may regulate or modulate the expression of specific genes or sequences in plant pathogens or pests, resulting in reduce pathogen pathogenicity and providing improved resistance in host plant.

In a further embodiment, a plant comprising a Disease Super Locus (DSL) disclosed herein may be stacked with one or more additional Bt insecticidal toxins, including, but not limited to, a Cry3B toxin, a mCry3B toxin, a mCry3A toxin, or a Cry34/35 toxin. In a further embodiment, a plant comprising a DSL may be stacked with one or more additional transgenes containing these Bt insecticidal toxins and other Coleopteran active Bt insecticidal traits for example, event MON863, event MIR604, event 5307, event DAS-59122, event DP-4114, event MON 87411, and event MON88017. In some embodiments, a plant comprising a DSL may be stacked with MON-87429-9 (MON87429 Event); MON87403; MON95379; MON87427; MON87419; MON-00603-6 (NK603); MON-87460-4; LY038; DAS-06275-8; BT176; BT11; MIR162; GA21; MZDT09Y; SYN-05307-1; DP-23211, DP-915635, and DAS-40278-9.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. In some embodiments a heterologous sequence comprises a polynucleotide encoding a polypeptide that is from the same species in a different location, a “native gene.” In some embodiments, a heterologous sequence comprises a native gene and a sequence from a different species. In some embodiments, a DSL comprises at least two heterologous native genes and no polynucleotides a different species.

### IV. Maize Plant Cells, Plants, and Seeds

“Maize” refers to a plant of the Zea mays L. ssp. mays and is also known as “corn”. The use of “ZM” preceding an object described herein refers to the fact that the object is from Zea mays.

Maize plants, maize plant cells, maize plant parts and seeds, and maize grain having the modified RppK (Genomic DNA SEQ ID NO: 9; cDNA SEQ ID NO: 10; Protein SEQ ID NO: 11), Ht1 (Genomic DNA SEQ ID NO: 6; cDNA SEQ ID NO: 7; Protein SEQ ID NO: 8), NLB18 (Genomic DNA SEQ ID NO: 1; cDNA SEQ ID NO: 2 or 4; Protein SEQ ID NO: 3 or 5), NLR01 (Genomic DNA SEQ ID No: 27; cDNA SEQ ID NO: 28; Protein SEQ ID No: 29), NLR02 (Genomic DNA SEQ ID Nos: 24; cDNA SEQ ID NO: 25; Protein SEQ ID No: 26), RCG1 (cDNA SEQ ID Nos: 30; Protein SEQ ID No: 31), RCG1b (cDNA SEQ ID Nos: 32; Protein SEQ ID No: 33), PRR03 (Genomic DNA SEQ ID Nos: 34; cDNA SEQ ID NO: 35; Protein SEQ ID No: 36), PRR01 (cDNA SEQ ID NO: 37; Protein SEQ ID No: 38), NLR01 (Genomic DNA SEQ ID Nos: 39; cDNA SEQ ID NO: 40; Protein SEQ ID No: 41), or NLR04 (Genomic DNA SEQ ID Nos: 42; cDNA SEQ ID NO: 43; Protein SEQ ID No: 44), for example sequences disclosed herein are also provided.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, kernels, ears, cobs, husks, stalks, roots, root tips, anthers, and the like. Grain is intended to mean the mature seed produced by commercial growers for purposes other than growing or reproducing the species.

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the spirit of the embodiments or the scope of the appended claims.

### Example 1

### Designing a Suitable Locus for Genetic Engineering of Disease Resistance Traits in Maize

Several considerations were taken into account for defining and selecting a region of the maize genome suitable for the development of a disease super locus: ease of product assembly, molecular characteristics, and regulatory and stewardship aspects.

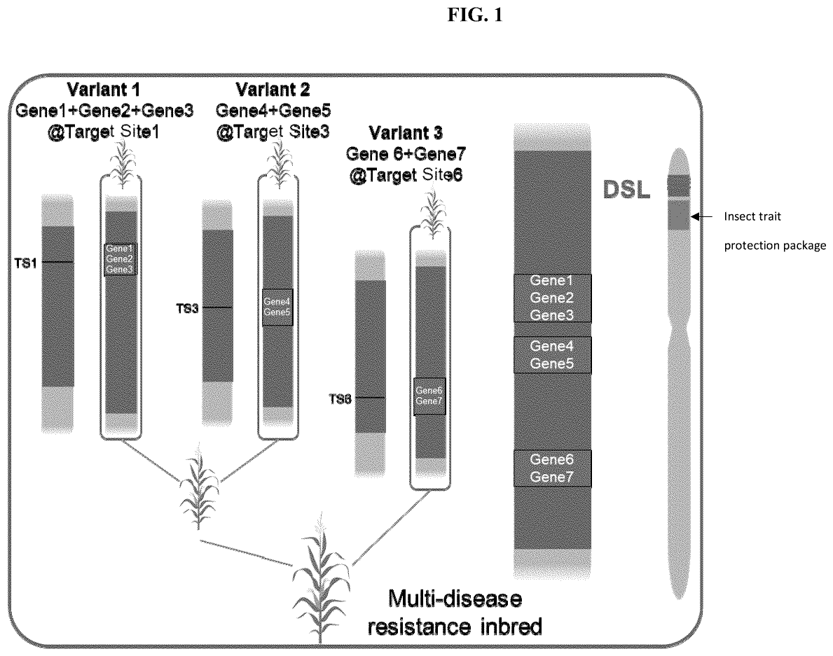

One selected locus, Disease Super Locus 1 (DSL1), is located in the distal region of chromosome 1 approximately 0.5 cM away from complex trait locus 1 (CTL1). This distance is specifically chosen and engineered to facilitate breeding stacks with inserted traits, such as insect control traits and/or herbicide tolerance traits inserted at CTL1 locus (in the manger illustrated by FIG. 1) and expedite final steps of product assembly. DSL1 spans approximately 3.2 cM or 515 Kbp in a region that does not display major structural variation across a range of germplasm, including a set of representative North American inbreds and a collection of tropical lines. At a more local level, pangenome alignment reveals that most of the region is structurally conserved in non-stiff stalk inbreds.

### Identification of Target Sites for Seamless Insertion of Traits

The DSL1 region was scanned for target sites using a bioinformatics tool searching for protospacer adjacent motifs (PAM) and retrieving the upstream 20-base sequences. The following filters were then applied to select the appropriate target sites and their corresponding guide RNAs.

Target sites were deemed unsuitable if less than 2.5 kb away from any native gene annotation. Gene annotations in the target inbred were based on a bioinformatic pipeline combining in silico predictions and in vivo evidence. For downstream analytical reasons, target sites located within 2 kb of repetitive regions larger than 200 bp were also deemed unsuitable.

Candidate guide RNAs targeting suitable sites were finally inspected in silico for their potential off-target activity using a bioinformatic tool run against the genome assembly. For each candidate guide RNA, a list of potential off-target sites was generated based on bioinformatics analysis, potential off-target hits were dismissed if they presented 3 or more mismatches with the guide including at least one mismatch in the PAM proximal seed sequence (Young, Zastrow-Hayes et al. 2019, Sci Rep April 30; 9(1):672).

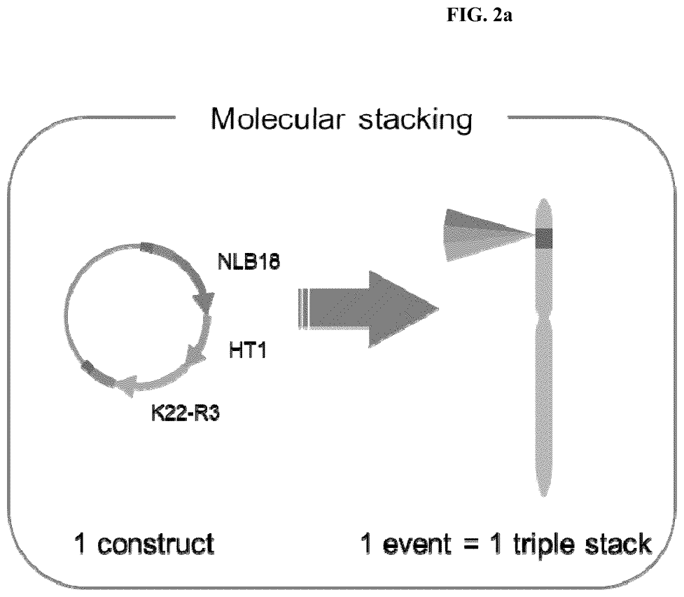

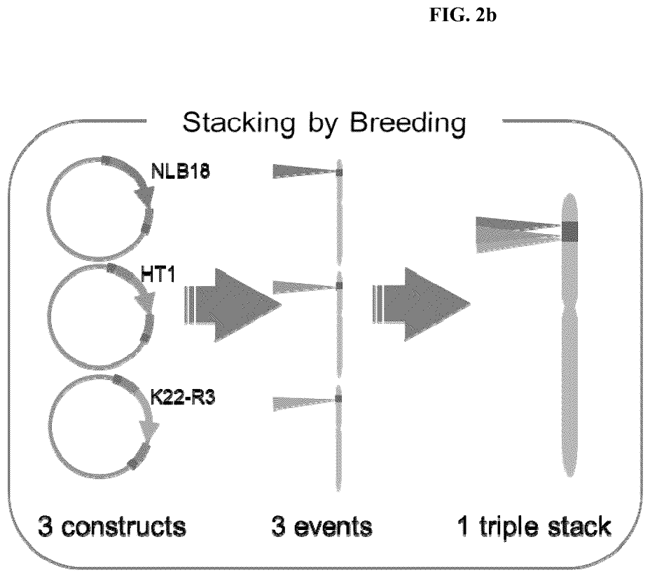

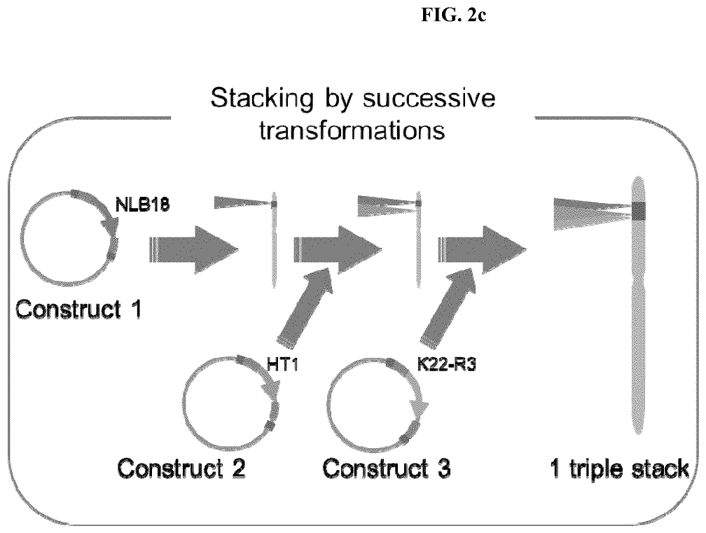

A list of potentially acceptable sites in DSL1 is provided in Tables 1 and 2. FIG. 2 shows a schematic drawing of the locations of target sites.

TABLE 3
Acceptable sites in DSL1

| | | | | Estimated |
|---|---|---|---|---|
| SEQ | | | | B73 genetic |
| ID NO: | Name | GUIDE_RNA_WITH_PAM | Best B73_v2 hit_Chr01 | coordinate |
| 12 | DSL1-CR1 | GCACGCTCCAGGTTAATGGCTGG | ZmChr1v2: 12883117 | 46.37 |
| 13 | DSL1-CR3 | GCAGCTGAAATTGAGCCTCCCGG | ZmChr1v2: 12917624 | 46.51 |
| 14 | DSL1-CR4 | GATTAGTCTCGGCATACGTACGG | ZmChr1v2: 12918033 | 46.51 |
| 15 | DSL1-CR5 | GGATAATGGCGTACGTATTGCGG | ZmChr1v2: 12921435 | 46.53 |
| 16 | DSL1-CR6 | GTTTCGAACAGAACGTACGCAGG | | |
| 17 | DSL1-CR7 | GGCTAGGCGTGTCACCATAATGG | ZmChr1v2: 12972339 | 46.74 |
| 18 | DSL1-CR9 | GAATACGAAACTATACCGCGGGG | | |
| 19 | DSL1-CR14 | GACTACCTCTGGGGGTACGTAGG | ZmChr1v2: 13502712 | 49.15 |
| 20 | DSL1-CR17 | GACGGGGACTTAATTATGCGTGG | ZmChr1v2: 13527536 | 49.28 |
| 21 | DSL1-CR18 | GCGATCCGTCACTTGTATATCGG | ZmChr1v2: 13550737 | 49.4 |

TABLE 4
Markers Flanking DSL1

| Probe/Marker | PHI_v2 cM | AlleleA Probe SEQ ID NO: |
|---|---|---|
| pze-101020971 | 45.75 | 22 |
| pze-101022341 | 49.45 | 23 |

### Vector Construction of Guides and Template

To improve their co-expression and presence, the Cas endonuclease and guide RNA expression cassettes were linked into a single DNA construct. A 480-490 bp sequence containing the guide RNA coding sequence, the 12-30 bp variable targeting domain from the chosen maize genomic target site, and part of the U6 promoter were synthesized. The sequence was then cloned to the backbone already having the cas cassette and the rest of the gRNA expression cassette.

Homology-directed repair (HDR) templates were designed to enable the insertion of disease resistance genes at the desired target sites. To optimize delivery, template sequences were synthesized and cloned on the vector backbone containing Cas endonuclease and guide RNA. In this setting, release of the template from the vector is achieved by inserting the target site sequence corresponding to the guide RNA encoded on the vector on each side of the HDR template FIG. 3). Template sequences included the full genomic region(s) of the disease resistance gene(s) of interest, flanked by homologous arms corresponding to the 100-1000 bp region directly adjacent to the cut site.

The plasmids comprising the Cas endonuclease expression cassette, guide RNA expression cassette and HDR template were delivered to maize embryos by Agrobacterium mediated transformation. Upon DNA cleavage at the designated site by Cas endonuclease, templates will be integrated by homology directed repair, resulting in seamless insertion at the cut site of the genomic regions conferring resistance to one or multiple diseases.

### Insertion of Maize Genomic Fragments Conferring Resistance Against Northern Leaf Blight and Southern Rust

One genomic fragment may contain a single source of resistance or multiple sources molecularly stacked to create genomic insertions at DSL1. In certain aspects, the coding sequences present within this genomic fragment are driven by their native regulatory sequences, such as native promoter and/or enhancer sequences compared to a transgenic cassette driven by a non-native or heterologous promoter. Single and stacked insertions at different target sites within DSL1 can be used individually or later combined by breeding. As an example, genomic fragments of NLB18 (Genomic DNA SEQ ID NO: 1; cDNA SEQ ID NO: 2 or 4; Protein SEQ ID NO: 3 or 5) or HT1 (Genomic DNA SEQ ID NO: 6; cDNA SEQ ID NO: 7; Protein SEQ ID NO: 8), conferring resistance against Northern Leaf Blight (U.S. patent application Ser. No. 16/341,531), and genomic fragment of RppK gene from inbred line K22 (WO2019/236257 (Genomic DNA SEQ ID NO: 9; cDNA SEQ ID NO: 10; Protein SEQ ID NO: 11), conferring resistance against Southern Rust, can be inserted at DSL1 individually or in combination as illustrated in FIG. 4.

### Example 2

Introgressing or Forward Breeding Multiple Disease Resistance Loci into Elite Germplasm

A Disease Super Locus (DSL) where multiple genes are combined within about a 5 cM region to confer resistance to multiple diseases can provide several advantages compared to independently introgressing of the different genes into a base inbred line.

To combine 7 genes from 7 different resistant donor lines conferring increased resistance to 4 different diseases, the number of populations that need to be developed to combine these QTL into a single inbred lines is large; and the different crosses that eventually are needed to move all loci containing the resistance gene into the same background are numerous and would take a long time. In addition, selecting for and maintaining 7 independent loci together in new crosses developed as part of a regular breeding programs would be commercially impractical and limits the number traits introduced in any given product cycle. This would require backcrossing the independent QTL regions into the same base inbred line that needs improvement for resistance. A typical scenario would include backcrossing and then selfing to obtain Near Isogenic Lines (NILs) with the locus containing the resistance gene present in the Recurrent Parent background.

Markers may be used to genotype for the presence of the resistance locus in the backcross lines and the subsequent selfed lines. A typical scenario is to develop a third backcross generation and two selfing (BC3S2) generation lines. If three generations can be grown per year, developing homozygous BC3S2 lines would take about 2 years.

Once Near Isogenic Lines for each of the individual seven loci have been developed, one would need to start making additional crosses to combine the 7 QTL regions, which will take additional generations (5-6 generations, which equals approximately 2 years) and large population sizes in order to be able to develop a Near Isogenic Line (NIL) with 7 homozygous resistance loci. To ensure these 7 loci are simultaneously selected for in subsequent breeding populations would require very large population sizes to ensure progeny containing seven homozygous loci would be obtained to maintain the desired level of resistance to multiple pathogens.

Theoretically, only 1 in 16384 progeny would be fixed for all seven loci in and F2 population derived from a line having all 7 resistance loci in homozygous from with a line not containing these 7 loci. This single progeny would only be selected for the presence of the 7 loci for resistance and not for any other desired traits. In a breeding program, many traits need to be considered when selecting the next generation of improved germplasm. Therefore, it could require, for example 30-100 F2 progeny containing the 7 resistance loci to also allow for the selection of other important traits that will be segregating in the F2 progeny of the two parents. This would translate to needing ˜0.5 million to ˜1.6 million progeny from one cross in order to ensure one can select a line that has both, improved agronomic traits and disease resistance at the 7 loci. Such population sizes are not possible in current commercial breeding programs.

Besides the extended time needed for the development of lines containing resistance loci from different donor sources and the enormous populations sizes needed to ensure presence of the 7 loci in subsequent generations, the other challenge will be to minimize linkage drag from the donor sources. Even when marker assisted selection is being used, recurrent parent genome recovery will be less than 100%. Even if only 2% of the donor source genome is retained in the recurrent parent background, this would translate into several hundreds of genes from the resistant donor parent being present in each of the Near Isogenic Lines developed for every single resistant locus.

When the resistance loci from 7 different Near Isogenic lines are brought together and assuming each of these NIL still contains 2% of their respective donor source genomes, the final Near Isogenic Line, containing the 7 resistant loci, may have up to 14% non-elite genome present in its background. Since resistant donor sources are often non-adapted lines, with good resistance but bad agronomic characteristics, the 14% derived from non-adapted donors will very likely result in detrimental effects on traits such as e.g. maturity and yield.

In contrast, using a DSL approach, the seven genes are transferred into a defined genomic region in a current elite germplasm line (or a select set of elite germplasm lines) selected for good agronomics. There will be little or no extra donor genome present in this line besides the genome fragment comprising the sequences for the seven disease resistant genes. In addition, this approximately 5 cM DSL region is identical or substantially identical in many commercially relevant elite lines and therefore introgression of this region into other elite lines will improve resistance to multiple pathogens.

The time frame for inserting the seven native resistance genes from different resistant maize donors into this elite line and developing the homozygous resistant lines is shorter using a DSL approach. Once such an initial resistant line, with exactly the same genomic background as the base inbred, besides the seven inserted genes within the 5 cM DSL region has been developed, it may be used as the resistant, elite bridge donor line for subsequent introgression of the DSL into other elite germplasm.

Such an introgression process may be finalized in a 2 year time frame and since the resistant bridge donor line is in an elite background, even if 2% of the genome of this resistant bridge donor line will still be present in the new introgressed line, there should be no negative effects on agronomic traits, since the bridge donor line is an elite line developed through many years of breeding for good agronomic characteristics.

### Opportunities for Breeding Programs Utilizing a DSL Region

Having the option to introgress or forward breed with the DSL region which confers resistance to multiple important pathogens, also allows breeding programs to utilize the rest of the genome for selection of favorable traits besides disease resistance. In otherwords, once the DSL region is fixed, breeders are free to choose, deselect, and/or select other linked or unliked traits to the previously located disease resistant loci without risking the loss of the resistance alleles due to segregation of desirable alleles. In the current breeding process, one always needs to select for a baseline of resistance for multiple diseases. Some of the regions involved with disease resistance may be linked to negative alleles for agronomic traits. If high levels of resistance to multiple pathogens can be brought in via introgression of, or forward breeding with the DSL, breeding programs can focus on selection for best agronomic traits utilizing all of the genomic regions outside of the DSL and will not have to compromise for disease resistance and putative linkage with negative effects in the rest of the genome. The opportunity to select desired agronomic characteristics utilizing all of the maize genome without being restricted to simultaneously select for a desired level resistance to multiple diseases, since the DSL provides such resistance, may result in quicker progress in breeding for traits such as for example yield, drought tolerance as well as other agronomic traits.

Improved Agronomic Traits with Multiple Disease Resistance with Reduced Yield Drag from Breeding

With the opportunity to select for positive agronomic traits across the genome, without the constraints of needing multiple different loci throughout the genome to confer a base level of resistance to multiple diseases, there is the potential to make additional progress in order to develop better yielding lines with better overall agronomics.

Replacing one or more resistance genes in the DSL of an elite lines containing such DSL may be necessary when the pathogen community in the field changes over the years, either due to a race shift that can overcome the resistance gene(s) or due to increasing problems with a new pathogen that was not a problem before.

Traditional crossing and selections to bring new QTL regions from non-adapted donor lines into elite germplasm is likely to be commercially costly due to the challenges mentioned around number of crosses, population size needed, timeline to develop inbred lines containing the combination of multiple QTLs in homozygous form as a disease control option. Keeping multiple QTL regions together in subsequent line germplasm development in the future is not currently feasible in regular breeding programs due to the same challenges.

In contrast, removing, replacing or adding new resistance genes to the DSL in an elite inbred line via the targeted gene editing technology is quicker and with reduced linkage drag around the gene of interest or due to background genetics coming from the resistant, non-adapted donor lines. This facilitates the development of an identical or a near identical line compared to the initial DSL containing inbred line but now with either new disease resistance genes replacing non-functional disease resistance genes, newly added disease resistance genes in the Disease Super Locus, or a new swapped DSL or a remodified DSL.

### Insertion of Multiple Copies of the Same Allele to Optimize Trait Expression and Eliminating Biparental Presence

In contrast to traditional crosses and selection procedures, multiple desired alleles of the same gene can be combined together in the DSL (i.e., in the same chromosomal arm/region) of one inbred line. This is sometimes desirable to confer the desired level of resistance. If two copies of a desired allele are present per chromosome at the DSL in the inbred line, then the hybrid resulting from a cross of this inbred line, with another inbred line (not having such allele) will result in a hybrid progeny with two copies of the allele. This would not be possible with traditional hybrid development, where one would need to introgress the gene of interest on both sides of the pedigree to develop a hybrid with two copies of the desired allele.

Stacking of Genetically Linked Resistance Genes from Multiple Sources

Resistance genes can be inserted into a DSL originating from different donor sources, but which are located in exactly the same region on the maize genome in those different donor lines. Using traditional crosses, combining such genes coming from different donor sources into one elite recurrent parent will be challenging or not practical for a commercial product development cycle due the fact that obtaining the correct recombination between genes in the same location on the genome from independent donor lines only occurs in very low frequencies. It would take large number of crosses and progeny to have a chance to identify a progeny line with the desired recombinations.

Stacking of Resistance Genes from Multiple Sources with Structural Variation Impeding Homologous Recombination

Maize contains disease resistance genes clusters, such as on the short arm of chromosome 10 (c10). These clusters can present significant structural variation, hindering homologous recombination during breeding crosses due to lack of sequence homology with other breeding lines.

Thus, to combine a disease resistance gene from donor line A on c10 with a disease resistance gene from donor line B that is located in the same genomic region on c10 and then move both disease resistance genes into elite inbred line C, can lead to several challenges. The c10 region can be genetically different between the three lines due to differences in gene content and intergenic sequence differences. It can potentially be difficult to obtain progeny (in a commercially relevant breeding cycle) that has any recombination in the c10 region since highly divergent regions are less likely to recombine. This reduces the opportunity to develop progeny in which the desired recombination produces the transfer of the two disease resistance genes from two different donor lines into an elite inbred line. In addition, even if one can successfully generate such a unique recombination, there is likely a large region from the donor lines that will still be present in the elite inbred line due to lack of recombination frequency and that results in linkage drag of donor line genome around the disease resistance genes into the elite inbred line. Linkage drag can result in the transfer of other undesirable genes that are adjacent or near the resistance gene cluster. Combining only the resistance alleles of different resistance genes from several inbred lines via recombination while simultaneously avoiding with the transfer of other undesirable susceptible alleles is often very difficult. By contrast, the Disease Super Locus disclosed herein will allow stacking of resistance alleles from multiple maize lines without the chance of introducing undesirable susceptible alleles through recombination, since transfer of a Disease Super Locus does not rely on recombination and creation of desired recombination, but allows for precise and targeted stacking of only the alleles that confer desired disease resistance.

### Insertion of DSL Locus in Proximity to Another Trait or Region of Interest

Another advantage of the development of a Disease Super Locus is that one may have this DSL be located immediately next to the genetic region in which an insect resistance locus (IRL) has been developed. In one embodiment, the IRL may be an Insect Super Locus (ISL). This will allow for simultaneous introgression of multiple insect resistance traits and disease resistance traits at the same time. The trait introgression process will be cost effective, since these multiple traits will be introgressed as one locus, it will be faster since there will be no need to introgress different loci in a recurrent parent and then make final crosses and self for several generations, to develop homozygous lines for both the insect resistance locus (IRL) and Disease Super Locus; and lastly, it will limit the presence of donor line genetics in the genomic background of the converted recurrent parent since only one Super Locus instead of two different Super Loci will be introgressed from a donor parent, which would result in a lower percentage linkage drag and lower percentage background genome from the donor parent present in the final introgressed line.

If there is a need or desire to separate the Insect Super Locus from the Disease Super Locus, this can be done by identifying recombinants between the two Super Loci (SL). A current line was created with a DSL about 0.6 cM genetic distance from an IRL. These SL were developed in elite germplasm, and the sequence similarity in this 0.6 cM region between the line containing the two SL and a large portion of other elite inbred lines is exactly the same. Therefore, the predicted recombination frequency is normal and recombinant progeny lines in an F2 populations are expected at a frequency of 1 in 165 progeny.

If there is a need or desire to separate the IRL from the Disease Resistance Trait Package in the DSL this can be done in these lines. Having the opportunity to introgress such combined trait packages at one locus, being able to separate the different trait packages as needed and being able to replace or add new disease resistance genes to the DSL region via gene editing, provides a very efficient method development of hybrids that are highly customized for specific environments.

Developing a distinct single SL that contains trait packages that allow for control of multiple diseases, or different insects or a combination of both also simplifies the process of combining such SL together with other traits, e.g. herbicide or insect tolerance, in a single hybrid. In a particular example, the DSL plus an insect control locus (“ISL”) is introgressed on the male side of the pedigree and combined with a herbicide tolerance trait on the female side of the pedigree. By limiting the number of loci to introgress, the SL more easily facilitates the combination another trait with this SL in one line. The number of progeny and crosses needed to develop a line that combines two independent loci of interest is orders of magnitude less compared to bringing 7 or more independent loci together in homozygous state in one single inbred line.

### Example 3

### Creation of DSL Locus in Proximity to Another Trait or Region of Interest

Creation of a disease super locus (DSL) at CRISPR sites near the locations utilized for transgenic insertion of Cry1B, Cry1D, DvSnf7, Cry75A and/or Vip4Da2 enables linking disease resistance traits to insect control tratis. Due to the prevalence of PAM sites in the genome, locations are chosen which result in varying levels of linkage between these two traits.

A DSL inserted at a location 20-30 cM away from a transgenic insertion of Cry1B, Cry1D, DvSnf7, Cry75A and/or Vip4Da2 enables the traits in the DSL to be linked to the insect control transgene event during introgression and passed on to a majority of introgrssion progeny, while also being separable from the transgene event in some progeny.

Placement of the DSL inserted at a location 10 cM, 5 cM, 4 cM, 3 cm, 2 cM, or 1 cM away from insect control events for each of Cry1B, Cry1D, DvSnf7, Cry75A and/or Vip4Da2 results in tight linkage and significantly reduces the burden of trait introgression, consistently producing maize lines that have both disease and insect resistance.

Thus, a DSL is inserted on the same chromosome arm as transgenic corn event MON95379 described in U.S. Patent Publication No. 2020/0032289A1. The DSL is inserted at a location of about 30 cM, at a location of about 20 cM, or at a location of about 15, 10, 5, 4, 3, 2, or 1 cM from transgenic corn event MON95379. In one embodiment, the DSL is inserted at a location of less than about 1 cM from transgenic corn event MON95379. Seeds comprising corn event MON95379 can be obtained from ATCC under Accession No. PTA-125027.

In a different example, a DSL is inserted on the same chromosome arm as transgenic corn event MON95275 described in U.S. Patent Publication No. 2021/0332380 A1. The DSL is inserted at a location of about 30 cM, at a location of about 20 cM, or at a location of about 15, 10, 5, 4, 3, 2, or 1 cM from transgenic corn event MON95275. In one embodiment, the DSL is inserted at a location of less than about 1 cM from transgenic corn event MON95275. Seeds comprising corn event MON95275 can be obtained from ATCC under Accession No. PTA-126049.

### Example 4

Short Stature Maize Plants Containing Genetic Modifications that Impact Plant Height

This example describes maize plants that are of short stature and comprise a DSL. See US20200199609A1, incorporated herein by reference in its entirety, which mentions methods and compositions to generate short stature plants and related agronomic management solutions. These DSL maize plants comprise one or more genetic modifications at genomic loci that are involved in plant height reduction. The DSL containing maize plant has a height that is reduced by about 5% to about 30% compared to the height of a control plant that lacks the genetic modification at the plant height loci; and/or the DSL-containing plant comprises an average leaf length to width ratio that is reduced at V6-V8 growth stages as compared to the control plant. In an embodiment, the plant height reduction does not substantially affect flowering time. In a further or separate embodiment, the flowering time does not change by more than about 5-10 CRM or plus or minus 10% GDU or 125-250 GDU, compared to a control plant not comprising the modifications.

### Brachytic (BR) Gene

A genomic sequence of the Br2 gene was identified and deposited under GenBank Accession No. AY366085. Multani et al. (2003) Science, 302(5642)81-84. Subsequently, Pilu et al. reported a br2-23 allele having an 8-bp deletion in the 3′ end of the Br2 gene and cairned a direct relationship between this deletion and the brachytic phenotype in their br2-23 plants. See Pilu et al., Molecular Breeding, 20:83-91(2007) In maize, brachytic mutants show a short stature due to a shortening of the internode length without a corresponding reduction in the number of internodes or the number and size of other organs, including the leaves, ear and tassel. See Kempton (1920) J. Hered., 11:111-115; Pilu et al. (2007) Molecular Breeding, 20:83-91 BR genes are plant hormones that regulate a number of major plant growth and developmental processes. A number of brachytic br mutants have been identified in maize to date, such as brachytic1 (br1), brachytic2 (br2), brachytic3 (br3), and edited BR2 mutant described in US20200140874A1, for example.

A DSL is inserted on the same chromosome arm as (e.g., within about 30 cM, about 20 cM, about 15, about 10, about 5, about 4, about 3, about 2, or about 1 cM of) a Br2 genomic locus disclosed in US20200199609A1, which is incorporated by reference herein in its entirety. The plant comprises a polynucleotide that encodes a Br2 polypeptide comprising an amino acid sequence that is at least 95% identical to SEQ ID NO: 43 of US20200199609A1, such that the edit results in results in (a) reduced expression of a polynucleotide encoding the Br2 polypeptide; (b) reduced activity of the Br2 polypeptide; (c) generation of one or more alternative spliced transcripts of a polynucleotide encoding the Br2 polypeptide; (d) deletion of one or more domains of the Br2 polypeptide; (e) frameshift mutation in one or more exons of a polynucleotide encoding the Br2 polypeptide; (f) deletion of a substantial portion of the polynucleotide encoding the Br2 polypeptide or deletion of the polynucleotide encoding the Br2 polypeptide; (g) repression of an enhancer motif present within a regulatory region encoding the Br2 polypeptide; (h) modification of one or more nucleotides or deletion of a regulatory element operably linked to the expression of the polynucleotide encoding the Br2 polypeptide, wherein the regulatory element is present within a promoter, intron, 3′UTR, terminator or a combination thereof.

### Gibberellic Acid Oxidase Genes

Gibberellins (gibberellic acids or GAs) are plant hormones that regulate a number of major plant growth and developmental processes. Manipulation of GA levels in semi-dwarf wheat, rice and sorghum plant varieties led to increased yield and reduced lodging in these cereal crops during the 20^{th} century, which was largely responsible for the Green Revolution. Certain biosynthetic enzymes (e.g., GA20 oxidase and GA3 oxidase) and catabolic enzymes (e.g., GA2 oxidase) in the GA pathway are critical to affecting active GA levels in plant tissues. Several of the GA oxidases in cereal plants consist of a family of related GA oxidase genes. For example, corn has a family of at least nine GA20 oxidase genes that includes GA20 oxidase_1, GA20 oxidase_2, GA20 oxidase_3, GA20 oxidase_4, GA20 oxidase_5, GA20 oxidase_6, GA20 oxidase_7, GA2) oxidase_8, and GA20 oxidase_9. (See US20210040498A1, WO2020243363A1, and US20210032646A1) There are two GA3 oxidases in corn, GA3 oxidase_1 and GA3 oxidase_2. (See 1US20210040498A1 and US20210032646A1.)

Thus, a DSL maize plant is created by inserting a DSL on the same chromosome arm as (e.g., within about 30 cM, about 20 cM, about 15, about 10, about 5, about 4, about 3, about 2, or about 1 cM of) a nucleotide change at the D8 genomic locus that comprises a gibberellic acid biosynthesis or signaling pathway. The altered D8 genetic locus can be one that produces (a) reduced expression of a polynucleotide encoding the D8 polypeptide (as represented by SEQ ID NO: 76 of US20200199609A1, incorporated herein by reference in its entirety; (b) reduced activity of the D8 polypeptide; (c) generation of one or more alternative spliced transcripts of a polynucleotide encoding the D8 polypeptide; (d) deletion of one or more domains of the D8 polypeptide; (e) frameshift mutation in one or more exons of a polynucleotide encoding the D8 polypeptide; (f) deletion of a substantial portion of the polynucleotide encoding the D8 polypeptide or deletion of the polynucleotide encoding the Br2 polypeptide; (g) repression of an enhancer motif present within a regulatory region encoding the D8 polypeptide; (h) modification of one or more nucleotides or deletion of a regulatory element operably linked to the expression of the polynucleotide encoding the D8 polypeptide, wherein the regulatory element is present within a promoter, intron, 3′UTR, terminator or a combination thereof.

The short stature DSL-containing plants disclosed herein can be planted at a higher planting density. In particular examples, the DSL-containing corn plants are planted at a density of about 30,000 to about 75,000 plants per acre. The planting density can also be at least 50,000 plants, 55,000 plants, 58,000 plants, 60,000 plants, 62,000 plants, or about 64,000 plants per acre. These can be planted in a plurality of rows having a row width of about 8 inches to about 30 inches.

Attenuating the Effects of Altered Hormone Levels in Short Stature Mutants with DSL

In another example, many reduced stature mutants result from alterations in hormone signaling pathways. For example, GA20-1 results from a loss of function of a gibberellin (GA) biosynthetic enzyme that causes short stature. GA has been implicated in regulating defense responses to a variety of plant pathogens. In the case that short stature mutants have altered or reduced pathogen defense responses due to the short stature mutation, the addition of disease resistance genes in DSL may compensate for the altered or reduced pathogen defense responses in such short stature plants.

### Example 5

### Increased Modularity of a DSL Approach Compared to Traditional Pyramiding of Traits by Breeding

A Disease Super Locus approach provides an easier way to modulate the set of genes necessary to provide adequate resistance to disease in specific environments, or in specific germplasm. For example, the set of diseases that are likely to affect a corn crop depends largely on the geography: the risk of developing Corn Southern Rust is higher in the South East than in other areas in the US, while the risk of developing Gray Leaf Spot is higher in the US corn belt and the Atlantic states and the risk of developing Goss's wilt is higher in the US corn belt. In addition, race evolution in certain areas may lead to new races becoming prevalent in specific geographies and spare other areas. It is also known that specific hybrid combinations are more or less susceptible to specific diseases or races, due to the underlying combination of native traits present in the inbred parents germplasms. Under these circumstances, it may be desirable to modulate the package of disease resistance traits that are delivered through the DSL and adapt it to specific geographies and germplasm susceptibilities. A super locus approach lends itself well to this need for flexibility that a traditional breeding approach can only achieve with methods that require significant time and dedicated effort. For example, a corn hybrid may present agronomic characteristics that make it well suited to multiple geographies with varying degrees of disease pressure. Using a DSL approach, one can readily insert the desired set of disease resistance genes in one inbred parent providing adequate resistance to disease most likely to occur in one area and a slightly different set of disease resistance genes in the same inbred parent providing adequate resistance to disease most likely to occur in another area. As a result, hybrids that present similar agronomic characteristics but disease resistance profiles that are adapted to distinct geographies can be produced using this approach. This can be achieved using a super locus approach by inserting two different sets of genes at DSL target sites. It can also be achieved by creating a first DSL insertion comprised of disease resistance genes against disease 1 “set 1” and then crossing with another inbred comprised of disease resistance genes against disease 2 “set 2”, while also crossing “set 1” with an inbred comprised of a third set of genes against disease 3 “set 3”, creating two inbreds each with different sets of resistance genes (sets 1 and 2, or sets 1 and 3). The same outcome can be achieved by creating a first inbred comprised of set 1, and re-transforming this inbred to create insertions of sets 2 or 3. It can also be achieved by creating a first inbred comprised of sets 1 and 2, and swapping set 2 with set 3. If one of the genes or sets of genes in an inbred created in one of these possible manners becomes obsolete because of shifting disease pressure for example, one could directly delete the unwanted gene or sets of genes, or swap it to replace with a more relevant gene or set of genes. In comparison, achieving the same outcome using traditional breeding methods would be impractical due to the cost and time required, as well as the potential for linkage drag occurring for each of the new genes introgressed. Such modularity can also be achieved by built-in, unique recombination linking (“URL”) sequences that are interspaced within a plurality of the disease resistance genes in a given DSL. For example, such a DSL can include a signature comprising “Resistance Gene A—URL1-Resistance Gene B-URL2-Resistance Gene C and so on and so forth. Such URLs can be designed to be targeted by specific recombination enhancing agents such as CRISPR-Cas endonucleases or any other site directed agent including for example, FLP/FRT recombinase based systems.

### Example 6

### Planting Density of DSL Plants

Pathogens are generally very sensitive to weather conditions. In addition, some pathogens are especially sensitive to the micro-environment in the plant canopy. High humidity may favor the growth of many different maize pathogens, including Cercospora maydis, Exserohilum turcicum and others. For example, humidity on and around leaf surface is conducive for the development of Gray Leaf Spot, which is caused by Cercospora maydis. It is expected that plant density and row spacing for example have a direct impact on this micro-environment. Higher density creates conditions where moisture is increased and ventilation is decreased, both amenable to pathogen development. The use of hybrids comprising DSL and resistant to GLS, for example, can mitigate this issue, and in turn enable higher planting densities (e.g., 40,000-80,000 or more maize plants per acre) which may otherwise not have been considered due to a higher risk of disease outbreak.

### Example 7

Maturity and Planting Date of DSL Plants

It is recognized that later maturity hybrids and delayed plantings are at higher risk of developing disease late in the season and incurring significant yield losses. Hybrids comprising a DSL and resistance to multiple diseases including those developing later in the growing season are expected to perform better when disease pressure is high during grain fill. Multi disease resistance brought by the presence of a disease super locus in the germplasm may provide more flexibility in planting date and enhanced yield protection for later maturity hybrid classes.

### Example 8

Overreliance on a single gene resistance may result in the selection of pathogen populations that can break the resistance. Disclosed herein is a gene-rotation strategy that employs planting varieties that carry resistance gene(s) or allele(s) with different modes of action than the gene(s) or allele(s) in the variety planted the previous season. By doing so, the pathogen population is exposed to different set of genes/alleles from season to season; this will reduce selection pressure on the pathogen. Gene rotation can be coupled with typical crop rotation to a non-host crop to further lengthen the rotation cycle and further reduce selection pressure to thereby prevent development of disease resistance in the pathogen.

Some disease epidemics start at a given location (latitude) and the pathogen can develop resistance to the mode of action (MOA) present in the locally grown crop. These resistant pathogens can be spread farther as the season progresses via wind or other mechanisms. An example of such phenomenon is Southern Corn Rust epidemics in the US which can start in the southern states and progressively move to northern states during the season. Spread of such disease across latitudes can be stopped by planting varieties with different modes of action across different latitudes. Similarly, planting a resistant variety at latitudes where epidemics appear first, can stop spread of disease to other latitudes, making it possible to plant susceptible varieties in other latitudes.

These strategies have previously not been commercially feasible due to lack of varieties that provide similar agronomic properties except for the disease resistance gene(s) they carry. Producing such varieties using breeding is extremely difficult due to crossovers and linkage drag associated with varietal development. The DSL plants and methods disclosed herein can be used to create varieties that differ only in the resistance gene(s) of interest but otherwise provide similar agronomic properties.

What is claimed is:

1. A method of generating a heterologous genomic locus in a transgenic plant or cell thereof, the method comprising introducing two or more intraspecies polynucleotide sequences to a predetermined locus thereby creating a heterologous genomic locus in the plant or cell thereof, wherein
(a) the introducing step does not result in integration of another transgene or a foreign polynucleotide that is not native to the plant or cell thereof;
(b) the intraspecies polynucleotides confer one or more agronomic characteristics to the plant or cell thereof;
(c) one or more of the intraspecies polynucleotides are from different chromosomes or the intraspecies polynucleotides are not located in their native chromosomal configuration prior to their integration into the heterologous genomic locus;
(d) the introducing step comprises at least one site-directed genome modification that is not done by conventional breeding; and
(e) the plant or cell thereof further comprises a first genomic locus which confers a transgenic trait, and the heterologous genomic locus is on the same chromosome arm as the transgenic genomic locus.

2. The method of claim 1, wherein the first transgenic locus comprises an insect control transgene, an herbicide tolerance transgene or transgenes conferring both insect control and herbicide tolerance traits.

3. The method of claim 1, wherein the first transgenic locus comprises a transgene encoding a fern-derived pesticidal protein, Bacillus thurigiensis (Bt)-derived pesticidal protein, a bacterial-derived pesticidal protein, or an RNA-based insect control agent.

4. The method of claim 1, wherein the first transgenic locus comprises a transgene encoding Cry1B, Cry1D, DvSnf7, Cry75A, or Vip4Da2.

5. The method of claim 1, wherein the heterologous genomic locus is located within 30 cM of the first genomic locus conferring the transgenic trait.

6. The method of claim 1, wherein the heterologous genomic locus is located within 10 cM of the first genomic locus conferring the transgenic trait.

7. The method of claim 1, wherein the heterologous genomic locus is located within 5 cM of the first genomic locus conferring the transgenic trait.

8. The method of claim 1, wherein the heterologous genomic locus is located within 1 cM of the first genomic locus conferring the transgenic trait.

9. The method of claim 1, wherein the first genomic locus comprises transgenic event MON95379.

10. A method of generating a heterologous genomic locus in a short stature plant or cell thereof, the method comprising introducing two or more intraspecies polynucleotide sequences to a predetermined locus thereby creating a heterologous genomic locus in the plant or cell thereof, wherein
(a) the introducing step does not result in integration of another transgene or a foreign polynucleotide that is not native to the plant or cell thereof;
(b) the intraspecies polynucleotides confer one or more agronomic characteristics to the plant or cell thereof;
(c) one or more of the intraspecies polynucleotides are from different chromosomes or the intraspecies polynucleotides are not located in their native chromosomal configuration prior to their integration into the heterologous genomic locus;
(d) the introducing step comprises at least one site-directed genome modification that is not done by conventional breeding; and
(e) the plant or cell thereof further comprises a first genomic locus which confers a short stature trait, and the heterologous genomic locus is on the same chromosome arm as the first genomic locus.

11. The method of claim 10, wherein the heterologous genomic locus is located within 30 cM of the first genomic locus conferring the short stature trait.

12. The method of claim 10, wherein the heterologous genomic locus is located within 10 cM of the first genomic locus conferring the short stature trait.

13. The method of claim 10, wherein the heterologous genomic locus is located within 5 cM of the first genomic locus conferring the short stature trait.

14. The method of claim 10, wherein the heterologous genomic locus is located within 1 cM of the first genomic locus conferring the short stature trait.

15. A method of generating a disease super locus in a plant genome, the method comprising introducing a plurality of disease resistance traits at a predetermined genomic locus of the crop plant chromosome by genomic modification that comprises (a) insertion of two or more disease resistant genes, (b) genomic translocation of one or more disease resistant genes through targeted chromosomal engineering, (c) duplication of one or more disease resistant genes at the genomic locus by targeted genome modification, (d) modifying the genomic locus by introducing one or more insertions, (e) deletions or substitutions of nucleotides in the genome, or (f) a combination of (a)-(e).

16. The method of claim 15, wherein the disease super locus is present in linkage disequilibrium with a transgenic locus that confers a insect control trait, an herbicide tolerance trait, or both insect control and herbicide tolerance traits

17. The method of claim 16, wherein the transgenic locus comprises a transgene encoding a fern-derived pesticidal protein, Bacillus thurigiensis (Bt)-derived pesticidal protein, a bacterial-derived pesticidal protein, or an RNA-based insect control agent.

18. The method of claim 15, wherein the disease super locus is present in linkage disequilibrium with transgenic event MON95379 or a transgene encoding one or more of Cry1B, Cry1D, DvSnf7, Cry75A, or Vip4Da2.

19. The method of claim 15, wherein the disease super locus is present in linkage disequilibrium with a locus conferring short stature trait.

20. The method of claim 15, further comprising introgressing the disease super locus into a plant comprising a transgenic event, the method comprising:
a. crossing the plant comprising the disease super locus with a second plant comprising at least one first genomic locus that confers a short stature trait or an insecticidal trait; and
b. obtaining a progeny plant comprising the disease super locus and the first genomic locus.

21. The method of claim 20, wherein introgression of the plurality disease resistance traits into the crop plant genome requires fewer backcrosses than conventional breeding.

22. A corn plant comprising a first genomic locus and a modified genomic locus, wherein
a. the first genomic locus confers a transgenic trait or short stature trait;
b. the modified genomic locus comprises at least a first modified target site and second modified target site;
c. the first modified target site comprises a first polynucleotide sequence that confers enhanced disease resistance to a first plant disease;
d. the second modified target site comprises a second polynucleotide sequence that confers enhanced disease resistance to the first plant disease or to a second plant disease;
e. the first and the second polynucleotide sequences are heterologous to the modified genomic locus and are each located less than about 1 cM from the other; and
f. the modified genomic locus and the first genomic locus are located on the same arm of a chromosome.

23. The plant of claim 22, wherein the modified genomic locus is located within about 30 cM from the first genomic locus.
